(19) United States
(12) Patent Application Publication
(10) Pub. No.: US 2025/0332152 A1
(43) Pub. Date: Oct. 30, 2025

(54) INHALED THERAPY FOR CARDIAC ARRHYTHMIA

(71) Applicant: InCarda Therapeutics, Inc., Newark, CA (US)

(72) Inventors: Luiz BELARDINELLI, Palo Alto, CA (US); Christopher DUFTON, Nederland, CO (US); Jean MAUPAS, Los Gatos, CA (US); Prashanti MADHAVAPEDDI, Fremont, CA (US)

(21) Appl. No.: 18/716,303

(22) PCT Filed: Dec. 8, 2022
(86) PCT No.: PCT/US2022/052294

§ 371 Date: Jun. 4, 2024

Related U.S. Application Data
(60) Provisional application No. 63/287,921, filed on Dec. 9, 2021.

(51) Int. Cl. A61K 31/4458; A61K 9/00; A61K 9/08; A61P 9/06

(52) U.S. Cl. CPC A61K 31/4458; A61K 9/0078; A61K 9/08; A61P 9/06

(57) ABSTRACT

The present disclosure relates to methods and kits for use in pharmacological treatment of cardiac arrhythmia (e.g., atrial fibrillation) in subjects with certain anthropometric characteristics, including overweight subjects.

### CROSS REFERENCE

This application claims the benefit of U.S. Provisional Patent Application No. 63/287,921, filed on Dec. 9, 2021, the content of which is incorporated by reference herein in its entirety.

### BACKGROUND

Cardiac arrhythmia (also dysrhythmia) is a term for any of a large and heterogeneous group of conditions in which there is abnormal electrical activity in the heart. The heart beat may be too fast or too slow and may be regular or irregular. Atrial arrhythmia therapy is a field with a high level of unmet clinical need. Many drugs used today have been on the market since the early 1980s and 1990s and are mostly inadequate due to either lack of efficacy or a cardiac side-effect profile that necessitates extensive monitoring of the patient.

There remains a need for improved compositions and methods for treating heart conditions, including methods that better take into account anthropometric factors to guide pharmacological treatment. Whether height, weight, and body mass index (BMI) are predictors of successful pharmacological cardioversion of atrial fibrillation is unknown.

### SUMMARY

In some embodiments, the present disclosure provides a method of treating cardiac arrhythmia, the method comprising:
(a) identifying a subject that: (i) is suffering from said cardiac arrhythmia, and (ii) has a qualifying body mass index (BMI), wherein said qualifying BMI is a BMI of no more than about 40 kg/m²;
(b) recommending administration to said subject via oral inhalation a pharmaceutical composition that comprises a therapeutically effective amount of an antiarrhythmic agent, wherein said recommendation is made at least on the basis that said subject has said qualifying BMI; and
(c) administering to said subject said pharmaceutical composition in accordance with said recommendation.

In some embodiments, the present disclosure provides a method of treating cardiac arrhythmia, the method comprising:
(a) identifying a subject that: (i) is suffering from said cardiac arrhythmia, and (ii) has a qualifying body mass index (BMI), wherein said qualifying BMI is a BMI of no more than about 40 kg/m²;
(b) determining that treatment of said subject is warranted at least on the basis that said subject has said qualifying BMI; and
(c) administering to said subject via oral inhalation a pharmaceutical composition that comprises a therapeutically effective amount of an antiarrhythmic agent in accordance with said determining.

In some embodiments, the present disclosure provides a method of treating cardiac arrhythmia, the method comprising:
(a) identifying a subject that: (i) is suffering from said cardiac arrhythmia, and (ii) has a qualifying body mass index (BMI), wherein said qualifying BMI is a BMI of no more than about 40 kg/m²;
(b) designating said subject as eligible for treatment at least on the basis that said subject has said qualifying BMI;
(c) administering to said subject via oral inhalation a pharmaceutical composition that comprises a therapeutically effective amount of an antiarrhythmic agent in accordance with said designating.

In some embodiments, the present disclosure provides a method of treating cardiac arrhythmia, the method comprising:
(a) identifying a subject that: (i) is suffering from said cardiac arrhythmia, and (ii) has a qualifying body mass index (BMI), wherein said qualifying BMI is a BMI of at least about 23 kg/m²;
(b) administering to said subject via oral inhalation a first dose of a pharmaceutical composition comprising an antiarrhythmic agent;
(c) after said administering of (b), waiting for a period of time, wherein said period of time is at least about 10 minutes;
(d) after (c), determining that said subject exhibits said cardiac arrhythmia after said period of time;
(e) after (d), recommending administration of a second dose of said pharmaceutical composition via oral inhalation to said subject, wherein said recommending is made based on at least: (i) said determining that subject exhibits said cardiac arrhythmia after said period of time; and (ii) that subject has said qualifying BMI; and
(f) administering said second dose of said pharmaceutical composition via oral inhalation to said subject in accordance with said recommendation.

In some embodiments, the present disclosure provides a method of treating cardiac arrhythmia, the method comprising:
(a) identifying a subject that: (i) is suffering from said cardiac arrhythmia, and (ii) has a qualifying body mass index (BMI), wherein said qualifying BMI is a BMI of at least about 23 kg/m²;
(b) administering to said subject via oral inhalation a first dose of a pharmaceutical composition comprising an antiarrhythmic agent;
(c) after the administering of (b), waiting for a period of time, wherein said period of time is at least about 10 minutes;
(d) after (c), determining that said subject exhibits said cardiac arrhythmia after said period of time;
(e) after the waiting, determining that administration of a second dose of said pharmaceutical composition via oral inhalation to said subject is warranted based on at least: (i) said determining that subject exhibits said cardiac arrhythmia after said period of time; and (ii) that subject has said qualifying BMI; and
(f) administering said second dose of said pharmaceutical composition via oral inhalation to said subject in accordance with said determining that administration of said second dose is warranted.

In some embodiments, the present disclosure provides a method of treating cardiac arrhythmia, the method comprising:
(a) identifying a subject that is: (i) suffering from said cardiac arrhythmia, and (ii) has a qualifying body mass index (BMI), wherein said qualifying BMI is a BMI of at least about 23 kg/m²;
(b) administering to said subject via oral inhalation a first dose of a pharmaceutical composition comprising an antiarrhythmic agent;
(c) after the administering of (b), waiting for a period of time, wherein said period of time is at least about 10 minutes,
(d) after (c), determining that said subject exhibits said cardiac arrhythmia after said period of time
(e) after the waiting, designating said subject as eligible for treatment with a second dose of said pharmaceutical composition via oral inhalation, wherein said designating is made based on at least: (i) said determining that subject exhibits said cardiac arrhythmia after said period of time; and (ii) that subject has said qualifying BMI;
(f) administering to said subject via oral inhalation said second dose of said pharmaceutical composition in accordance with said designating.

In some embodiments, the present disclosure provides a method of treating cardiac arrhythmia in a subject, wherein the method comprises:
(a) administering to said subject via oral inhalation a first dose of a pharmaceutical composition, wherein said pharmaceutical composition is an aqueous solution that comprises flecainide or a pharmaceutically acceptable salt thereof at a concentration that is at least 60 mg/ml;
(b) after the administering of (a), waiting for a period of time, wherein said period of time is at least about 10 minutes; and
(c) after the waiting, administering to said subject a second dose of said pharmaceutical composition via oral inhalation, wherein said second dose comprises said flecainide or salt thereof in an amount that is equivalent to about 40% to about 100% (w/w) of said first dose.

In some embodiments, the present disclosure provides a method of treating cardiac arrhythmia in a subject, wherein the method comprises:
(a) administering to said subject via oral inhalation a first dose of a pharmaceutical composition comprising from about 80 mg to about 160 mg flecainide or a pharmaceutically acceptable salt thereof, wherein said pharmaceutical composition is an aqueous solution of said flecainide or salt thereof;
(b) after the administering of (a), waiting for a period of time, wherein said period of time is at least about 10 minutes; and
(c) after the waiting, administering to said subject a second dose of said pharmaceutical composition via oral inhalation, wherein said second dose comprises said flecainide or salt thereof in an amount that is equivalent to about 40% to about 100% (w/w) of said first dose.

In some embodiments, the present disclosure provides a kit comprising a first dose and a second dose of a pharmaceutical composition formulated for oral inhalation, wherein:
(a) said pharmaceutical composition comprises a class I antiarrhythmic agent or a pharmaceutically acceptable salt thereof;
(b) said second dose comprises said class I antiarrhythmic agent or a pharmaceutically acceptable salt thereof in an amount that is equivalent to about 40% to about 80% (w/w) of said first dose;
(c) said first dose is provided in a first vessel, and said second dose is provided in a second vessel.

In some embodiments, the present disclosure provides a method of treating a heart condition, comprising:
identifying a human subject that has experienced or is experiencing said heart condition, and that has a body mass index (BMI) of no more than 40 kg/m²; and
administering to said subject via oral inhalation a pharmaceutical composition that comprises a therapeutically effective amount of an antiarrhythmic agent.

In some embodiments, the present disclosure provides a method of treatment of a heart condition, comprising administering to a human subject via inhalation a pharmaceutical composition that comprises a therapeutically effective amount of an antiarrhythmic agent, wherein said subject has been prescribed with said pharmaceutical composition based, at least in part, on that: (a) said subject has experienced or is experiencing said heart condition, and (b) said subject has a body mass index (BMI) of at most 40 kg/m².

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 is a chart depicting the relationship between body mass index (BMI) and height in atrial fibrillation patients that underwent cardioversion or did not undergo cardioversion by 90 minutes after oral inhalation of 120 mg flecainide acetate.

FIG. 2 is a chart depicting the relationship between body mass index (BMI) and flecainide Cₘₐₓ in atrial fibrillation patients that underwent cardioversion or did not undergo cardioversion by 90 minutes after oral inhalation of 120 mg flecainide acetate.

FIG. 3 is a chart depicting a boundary analysis of weight versus cardioversion rate in atrial fibrillation patients (n=88) administered 120 mg flecainide acetate via oral inhalation.

FIG. 4 is a chart depicting a lower boundary analysis of BMI versus cardioversion rate. in atrial fibrillation patients (n=81) administered 120 mg flecainide acetate via oral inhalation

FIG. 5 is a chart depicting an upper boundary analysis of BMI versus cardioversion rate in atrial fibrillation patients (n=88) administered 120 mg flecainide acetate via oral inhalation.

FIG. 6 is a chart depicting median Cₘₐₓ versus BMI classification (normal (BMI<25), overweight (BMI≥25 and <30), obese/severe obese (BMI≥30)) in atrial fibrillation patients (n=81) administered 120 mg flecainide acetate via oral inhalation.

### DETAILED DESCRIPTION

As an overview, the present disclosure relates to the treatment of cardiac arrhythmia (e.g., atrial fibrillation) in individuals with certain anthropometric characteristics, including patients that are overweight.

Atrial fibrillation (AF) is the most common cardiac arrhythmia, and it is associated with a significant impact on patient morbidity and mortality. Several cardiovascular (e.g., arterial hypertension) and non-cardiovascular (e.g., hyperthyroidism) diseases are risk factors for AF. Structural and electrical remodeling can create a substrate that promotes AF in taller and obese subjects. For example, progressive weight gain on AF substrate can lead to an increase in atrial volume, fibrosis, and inflammation accompanied by increased heterogeneity of atrial electrical conduction velocity (CV), inducibility, and spontaneous AF. In addition, subjects with lone AF and apparently no CV disease who are otherwise healthy can have larger atria than age-matched controls. In overweight and obese subjects, prolongation of PR interval and P wave duration can progressively increase with BMI (25-30 kg/m² and ≥30 kg/m²) compared with subjects with BMI<25 kg/m². In addition, progressive weight loss in overweight and obese subjects with symptomatic AF can resulted in a “dose-dependent” decrease in AF burden and left atrial volume.

The efficacy of Class Ic AADs (e.g., flecainide) to either acutely cardiovert episodes of AF or prevent recurrences of AF can be reduced in obese and taller patients. Notwithstanding the potential contribution of any “drug-dilution” due to the increase blood volume in heavier subjects, structural and EP remodeling of the atria could account to a significant extent to the reduced efficacy of class Ic AADs such as flecainide and propafenone to cardiovert and/or prevent recurrences of AF. For example, in obese subjects, electrophysiological and structural changes in atrial myocytes and tissue can be: 1) down-regulation of ion channels such as Na⁺ (Nav1.5), K+ (Kv1.5) and Ca²⁺ (Cav1.2) and the respective currents that flow through these channels (I_{Na}, I_{Kur}, I_{CaL}), 2) shortening of atrial action potential, 3) slowing and increase in heterogeneity of atria electrical CV, 4) decrease in the maximal rate of rise of action potential, 5) increase in atrial size, and 6) increases in atrial fibrosis and inflammation. All the above changes, EP and structural, can render atrial myocytes and tissue less sensitive to anti-AF electrophysiological effects of class Ic drugs. Specifically, adding flecainide to atria with reduced INa can further reduce CV and possibly increase heterogeneity of EP parameters, including dispersion of refractoriness thereby exerting proarrhythmic and/or perpetuation of AF. These EP changes together with an enlarged and fibrotic atria can form a substrate that can promote AF and also render it resistant to cardioversion or prevention of recurrences with AADs.

In view of the above, there is a need for a pharmacological treatment of atrial arrhythmia that can leverage anthropometric factors such as patient height and weight to improve efficacy of a drug.

As used herein, “heart condition” can refer to a condition where heart has an abnormal function and/or structure, for example, heart is beating in an irregular rhythm, experiencing arrhythmia, atrial fibrillation, and/or tachycardia, there is myocardial infarction, and/or coronary heart disease. As used herein, “atrial arrhythmia” can refer to an arrhythmia that affects at least one atrium and does not include bradycardia. For instance, atrial arrhythmia may originate in and affect at least one atrium. As used herein, “tachycardia” can refer to an arrhythmia in which the heart beat is too fast. For instance, tachycardia may involve a resting heart rate of over 100 beats per minute, such as greater than 110, greater than 120, or greater than 130 beats minute. In some cases, tachycardia can comprise sinus tachycardia, atrial fibrillation, atrial flutter, AV nodal reentrant tachycardia, accessory pathway mediated tachycardia, atrial tachycardia, multifocal atrial tachycardia, junctional tachycardia, ventricular tachycardia, supraventricular tachycardia, or any combination thereof.

As used herein, the phrase “heart rhythm arrhythmia” can refer to an arrhythmia in which the heart beat is irregular. As used herein, the term “atrial fibrillation” can refer to an abnormal heart rhythm characterized by rapid and irregular beating of the atria. As used herein, the term “cardioversion” can refer to a process by which an abnormally fast heart rate (tachycardia) or other cardiac arrhythmia is converted to a normal sinus rhythm. Cardioversion can be induced by electricity, drugs, or both.

As used herein, the singular forms “a,” “an,” and “the” can include plural referents unless the context clearly dictates otherwise. Thus, for example, reference to “an antiarrhythmic agent” can include not only a single active agent but also a combination or mixture of two or more different active agents.

Reference herein to “one embodiment,” “one version,” or “one aspect” can include one or more such embodiments, versions or aspects, unless otherwise clear from the context.

As used herein, the term “pharmaceutically acceptable solvate” can refer to a solvate that retains one or more of the biological activities and/or properties of the antiarrhythmic pharmaceutical agent and that is not biologically or otherwise undesirable. Examples of pharmaceutically acceptable solvates include, but are not limited to, antiarrhythmic pharmaceutical agents in combination with water, isopropanol, ethanol, methanol, DMSO, ethyl acetate, acetic acid, ethanolamine, or combinations thereof.

As used herein, the term “salt” is equivalent to the term “pharmaceutically acceptable salt,” and can refer to those salts that retain one or more of the biological activities and properties of the free acids and bases and that are not biologically or otherwise undesirable. Illustrative examples of pharmaceutically acceptable salts include, but are not limited to, sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, di nitrobenzoates, hydroxybenzoates, methoxybenzoates, phthalates, sulfonates, xylenesulfonates, phenylacetates, phenyipropionates, phenylbutyrates, citrates, lactates, γ-hydroxybutyrates, glycolates, tartrates, methanesulfonates, propanesulfonates, naphthalene-1-sulfonates, naphthalene-2-sulfonates, and mandelates.

The term “about” in relation to a reference numerical value can include a range of values plus or minus 10% from that value. For example, the amount “about 10” includes amounts from 9 to 11, including the reference numbers of 9, 10, and 11. The term “about” in relation to a reference numerical value can also include a range of values plus or minus 10%, 9%, 8%, 7%, 6%, 5%, 4%, 3%, 2%, or 1% from that value.

As used herein, “atrial arrhythmia” can refer to an arrhythmia that affects at least one atrium and does not include bradycardia. For instance, atrial arrhythmia can originate in and affect at least one atrium.

As used herein, “tachycardia” can mean an arrhythmia in which the heart beat is too fast, e.g., faster than normal. For instance, tachycardia may involve a resting heart rate of over 100 beats per minute, such as greater than 110, greater than 120, or greater than 130 beats minute.

As used herein, the phrase “heart rhythm arrhythmia” can refer to an arrhythmia in which the heart beat is irregular.

As used herein, the amount of an agent as described herein in the coronary circulation of the heart” can be measured by extracting a sample from any vascular region of the coronary circulation of the heart (e.g., arteries, veins, including coronary sinus) by using a cannula. The amount of the agent in the sample can then be determined by known means, such as bioanalytical techniques that employ analytical equipment such as LC-MS/MS. Thus, the amount of the agent in the blood in the heart can be measured for any particular time.

As used herein, the terms “treating” and “treatment” can refer to reduction in severity and/or frequency of symptoms, elimination of symptoms and/or underlying cause, reduction in likelihood of the occurrence of symptoms and/or underlying cause, and/or remediation of damage. Thus, “treating” a patient with an active agent as provided herein can include prevention of a particular condition, disease, or disorder in a susceptible individual as well as treatment of a clinically symptomatic individual.

As used herein, “nominal amount” can refer to the amount contained within the unit dose receptacle(s) that are administered.

As used herein, “effective amount” can refer to an amount covering both therapeutically effective amounts and prophylactically effective amounts.

As used herein, a “therapeutically effective amount” of an active agent can refer to an amount that is effective to achieve a desired therapeutic result. A therapeutically effective amount of a given active agent can vary with respect to factors such as the type and severity of the disorder or disease being treated and the age, gender, and weight of the patient. In some cases, “inhalation” (e.g., “oral inhalation” or “nasal inhalation”) refers to inhalation delivery of a therapeutically effective amount of a pharmaceutical agent contained in one unit dose receptacle, which, in some instance, can require one or more breaths, like 1, 2, 3, 4, 5, 6, 7, 8, 9, or more breaths. For example, if the effective amount is 90 mg, and each unit dose receptacle contains 30 mg, the delivery of the effective amount can require 3 inhalations.

Unless otherwise specified, the term “therapeutically effective amount” can include a “prophylactically effective amount,” e.g., an amount of active agent that is effective to prevent the onset or recurrence of a particular condition, disease, or disorder in a susceptible individual.

As used herein, the phrase “minimum effective amount” can mean the minimum amount of a pharmaceutical agent necessary to achieve an effective amount.

As used herein, “mass median diameter” or “MMD” can refer to the median diameter of a plurality of particles, typically in a polydisperse particle population, e.g., consisting of a range of particle sizes. MMD values as reported herein are determined by laser diffraction (Sympatec Helos, Clausthal-Zellerfeld, Germany), unless the context indicates otherwise. For instance, for powders the samples are added directly to the feeder funnel of the Sympatec RODOS dry powder dispersion unit. This can be achieved manually or by agitating mechanically from the end of a VIBRI vibratory feeder element. Samples are dispersed to primary particles via application of pressurized air (2 to 3 bar), with vacuum depression (suction) maximized for a given dispersion pressure. Dispersed particles are probed with a 632.8 nm laser beam that intersects the dispersed particles' trajectory at right angles. Laser light scattered from the ensemble of particles is imaged onto a concentric array of photomultiplier detector elements using a reverse-Fourier lens assembly. Scattered light is acquired in time-slices of 5 ms. Particle size distributions are back-calculated from the scattered light spatial/intensity distribution using a proprietary algorithm.

As used herein, “geometric diameter” can refer to the diameter of a single particle, as determined by microscopy, unless the context indicates otherwise.

As used herein, “mass median aerodynamic diameter” or “MMAD” can refer to the median aerodynamic size of a plurality of particles or particles, typically in a polydisperse population. The “aerodynamic diameter” can be the diameter of a unit density sphere having the same settling velocity, generally in air, as a powder and is therefore a useful way to characterize an aerosolized powder or other dispersed particle or particle formulation in terms of its settling behavior. The aerodynamic diameter encompasses particle or particle shape, density, and physical size of the particle or particle. As used herein, MMAD refers to the median of the aerodynamic particle or particle size distribution of aerosolized particles determined by cascade impaction, unless the context indicates otherwise.

By a “pharmaceutically acceptable” component is meant a component that is not biologically or otherwise undesirable, e.g., the component may be incorporated into a pharmaceutical formulation of the disclosure and administered to a patient as described herein without causing any significant undesirable biological effects or interacting in a deleterious manner with any of the other components of the formulation in which it is contained. When the term “pharmaceutically acceptable” is used to refer to an excipient, it can imply that the component has met the required standards of toxicological and manufacturing testing or that it is included on the Inactive Ingredient Guide prepared by the U.S. Food and Drug Administration.

As used herein, “P wave” can represent the wave generated by the electrical depolarization of the atria (right and left) and is usually 0.08 to 0.1 seconds (80-100 ms) in duration.

As used herein, “room temperature” can refer to a temperature that is from 18° C. to 25° C.

### Methods of Treatment

In one aspect of the present disclosure, provided is a method of treating a subject suffering from a heart condition. In some embodiments, the method comprises: administering to the subject via inhalation a pharmaceutical composition in the form of a liquid solution, wherein the pharmaceutical composition comprises a therapeutically effective amount of a salt of flecainide, and wherein a concentration of the salt of flecainide in the pharmaceutical composition is above 60 mg/mL.

In some embodiments, the present disclosure provides a method of treatment where a recommendation is made based on observation of a physiological state of the patient. A health care practitioner can recommend administration of a therapy, such as a pharmaceutical composition of the disclosure by, for example, consultation with the subject or other healthcare providers, or by entering the recommendation into a medical record.

In some embodiments, the pharmaceutical composition and the method of treatment provided herein are advantageous in offering fast, efficient, and safe therapeutic solution to heart conditions, such as cardiac arrhythmia, such as atrial arrhythmia. In some embodiments, the present disclosure relates to inhalation administration of a pharmaceutical composition in the form of a solution that comprises a salt of flecainide.

In some embodiments, the present disclosure provides a method of treating cardiac arrhythmia, the method comprising:
(a) identifying a subject that: (i) is suffering from said cardiac arrhythmia, and (ii) has a qualifying body mass index (BMI), wherein said qualifying BMI is a BMI of no more than about 40 kg/m²;
(b) recommending administration to said subject via oral inhalation a pharmaceutical composition that comprises a therapeutically effective amount of an antiarrhythmic agent, wherein said recommendation is made at least on the basis that said subject has said qualifying BMI; and
(c) administering to said subject said pharmaceutical composition in accordance with said recommendation.

In some embodiments, the present disclosure provides a method of treating cardiac arrhythmia, the method comprising:
(a) identifying a subject that: (i) is suffering from said cardiac arrhythmia, and (ii) has a qualifying body mass index (BMI), wherein said qualifying BMI is a BMI of no more than about 40 kg/m²;
(b) determining that treatment of said subject is warranted at least on the basis that said subject has said qualifying BMI; and
(c) administering to said subject via oral inhalation a pharmaceutical composition that comprises a therapeutically effective amount of an antiarrhythmic agent in accordance with said determining.

In some embodiments, the present disclosure provides a method of treating cardiac arrhythmia, the method comprising:
(a) identifying a subject that: (i) is suffering from said cardiac arrhythmia, and (ii) has a qualifying body mass index (BMI), wherein said qualifying BMI is a BMI of no more than about 40 kg/m²;
(b) designating said subject as eligible for treatment at least on the basis that said subject has said qualifying BMI;
(c) administering to said subject via oral inhalation a pharmaceutical composition that comprises a therapeutically effective amount of an antiarrhythmic agent in accordance with said designating.

In some embodiments, said subject has a systolic blood pressure that is greater than about 90 mmHg at initiation of said administering. In some embodiments, said subject has a systolic blood pressure that is from about 100 mmHg to about 180 mmHg at initiation of said administering. In some embodiments, said subject has a systolic blood pressure that is from about 100 mmHg to about 170 mmHg at initiation of said administering. In some embodiments, said subject has a systolic blood pressure that is from about 100 mmHg to about 160 mmHg at initiation of said administering.

In some embodiments, said subject has a ventricular rate that is no more than 170 BPM at initiation of said administering. In some embodiments, said subject has a ventricular rate that is from about 80 BPM to about 155 BPM at initiation of said administering. In some embodiments, said subject has a ventricular rate that is at least about 50 BPM, at least about 55 BPM, at least about 60 BPM, at least about 65 BPM, at least about 70 BPM, at least about 75 BPM, at least about 80 BPM, at least about 85 BPM, at least about 90 BPM, at least about 95 BPM, or at least about 100 BPM at initiation of said administering. In some embodiments, said subject has a ventricular rate that is no greater than about 200 BPM, no greater than about 190 BPM, no greater than about 180 BPM, no greater than about 175 BPM, no greater than about 170 BPM, no greater than about 165 BPM, no greater than about 160 BPM, no greater than about 155 BPM, no greater than about 150 BPM, no greater than about 145 BPM, or no greater than about 140 BPM at initiation of said administering.

In some embodiments, said qualifying BMI is a BMI of no more than about 37 kg/m², about 35 kg/m², about 33 kg/m², about 31 kg/m², about 30 kg/m², about 29 kg/m², about 27 kg/m², or about 25 kg/m². In some embodiments, said qualifying BMI is a BMI of no more than about 37 kg/m². In some embodiments, said qualifying BMI is a BMI of no more than about 36 kg/m². In some embodiments, said qualifying BMI is a BMI of no more than about 35 kg/m². In some embodiments, said qualifying BMI is a BMI of no more than about 34 kg/m². In some embodiments, said qualifying BMI is a BMI of no more than about 33 kg/m².

In some embodiments, said antiarrhythmic agent is a class I, II, III, IV, or V antiarrhythmic agent.

In some embodiments, the present disclosure provides a method of treating cardiac arrhythmia, the method comprising:
(a) identifying a subject that: (i) is suffering from said cardiac arrhythmia, and (ii) has a qualifying body mass index (BMI), wherein said qualifying BMI is a BMI of at least about 23 kg/m²;
(b) administering to said subject via oral inhalation a first dose of a pharmaceutical composition comprising an antiarrhythmic agent;
(c) after said administering of (b), waiting for a period of time, wherein said period of time is at least about 10 minutes;
(d) after (c), determining that said subject exhibits said cardiac arrhythmia after said period of time;
(e) after (d), recommending administration of a second dose of said pharmaceutical composition via oral inhalation to said subject, wherein said recommending is made based on at least: (i) said determining that subject exhibits said cardiac arrhythmia after said period of time; and (ii) that subject has said qualifying BMI; and
(f) administering said second dose of said pharmaceutical composition via oral inhalation to said subject in accordance with said recommendation.

In some embodiments, the present disclosure provides a method of treating cardiac arrhythmia, the method comprising:
(a) identifying a subject that: (i) is suffering from said cardiac arrhythmia, and (ii) has a qualifying body mass index (BMI), wherein said qualifying BMI is a BMI of at least about 23 kg/m²;
(b) administering to said subject via oral inhalation a first dose of a pharmaceutical composition comprising an antiarrhythmic agent;
(c) after the administering of (b), waiting for a period of time, wherein said period of time is at least about 10 minutes;
(d) after (c), determining that said subject exhibits said cardiac arrhythmia after said period of time;
(e) after the waiting, determining that administration of a second dose of said pharmaceutical composition via oral inhalation to said subject is warranted based on at least: (i) said determining that subject exhibits said cardiac arrhythmia after said period of time; and (ii) that subject has said qualifying BMI; and
(f) administering said second dose of said pharmaceutical composition via oral inhalation to said subject in accordance with said determining that administration of said second dose is warranted.

In some embodiments, the present disclosure provides a method of treating cardiac arrhythmia, the method comprising:
(a) identifying a subject that is: (i) suffering from said cardiac arrhythmia, and (ii) has a qualifying body mass index (BMI), wherein said qualifying BMI is a BMI of at least about 23 kg/m²;
(b) administering to said subject via oral inhalation a first dose of a pharmaceutical composition comprising an antiarrhythmic agent;
(c) after the administering of (b), waiting for a period of time, wherein said period of time is at least about 10 minutes,
(d) after (c), determining that said subject exhibits said cardiac arrhythmia after said period of time

(e) after the waiting, designating said subject as eligible for treatment with a second dose of said pharmaceutical composition via oral inhalation, wherein said designating is made based on at least: (i) said determining that subject exhibits said cardiac arrhythmia after said period of time; and (ii) that subject has said qualifying BMI;

(f) administering to said subject via oral inhalation said second dose of said pharmaceutical composition in accordance with said designating.

In some embodiments, said determining of (d) comprises obtaining an ECG of said subject.

In some embodiments, said period of time is at least about 20 minutes, at least about 30 minutes, at least about 40 minutes, at least about 50 minutes, at least about 60 minutes, at least about 70 minutes, at least about 80 minutes, at least about 90 minutes, at least about 100 minutes, at least about 110 minutes, or at least about 120 minutes. In some embodiments, said period of time is at least about 15 minutes. In some embodiments, said period of time is at least about 20 minutes. In some embodiments, said period of time is at least about 30 minutes.

In some embodiments, said qualifying BMI is a BMI of at least about 25 kg/m², 27 kg/m², about 29 kg/m², about 30 kg/m², about 31 kg/m², about 33 kg/m², about 35 kg/m², about 37 kg/m², or about 40 kg/m². In some embodiments, said qualifying BMI is a BMI of at least about 25 kg/m². In some embodiments, said qualifying BMI is a BMI from about 23 kg/m² to about 40 kg/m². In some embodiments, said qualifying BMI is a BMI from about 23 kg/m² to about 37 kg/m². In some embodiments, said qualifying BMI is a BMI from about 23 kg/m² to about 35 kg/m². In some embodiments, said qualifying BMI is a BMI from about 25 kg/m² to about 40 kg/m². In some embodiments, said qualifying BMI is a BMI from about 25 kg/m² to about 37 kg/m². In some embodiments, said qualifying BMI is a BMI from about 25 kg/m² to about 35 kg/m². In some embodiments, said qualifying BMI is a BMI from about 25 kg/m² to about 30 kg/m². In some embodiments, said qualifying BMI is a BMI from about 30 kg/m² to about 40 kg/m². In some embodiments, said qualifying BMI is a BMI from about 25 kg/m² to about 40 kg/m².

In some embodiments, said identifying of (a) further comprises determining said BMI of said subject. In some embodiments, said determining said BMI comprises determining subject height and body mass. In some embodiments, said subject height or body mass is reported by said subject. In some embodiments, said subject height or body mass is measured by a health practitioner.

In some embodiments, said subject has a systolic blood pressure that is greater than about 90 mmHg at the time of treating. In some embodiments, said subject has a systolic blood pressure that is from about 100 mmHg to about 160 mmHg at the time of treating.

In some embodiments, said subject has a ventricular rate that is no more than 170 BPM at the time of treating. In some embodiments, said subject has a ventricular rate that is from about 80 BPM to about 155 BPM at the time of treating.

In some embodiments, said subject has a ventricular rate that is no more than 170 BPM at initiation of said administering of (b). In some embodiments, said subject has a ventricular rate that is from about 80 BPM to about 155 BPM at initiation of said administering of (b). In some embodiments, said subject has a ventricular rate that is at least about 50 BPM, at least about 55 BPM, at least about 60 BPM, at least about 65 BPM, at least about 70 BPM, at least about 75 BPM, at least about 80 BPM, at least about 85 BPM, at least about 90 BPM, at least about 95 BPM, or at least about 100 BPM at initiation of said administering of (b). In some embodiments, said subject has a ventricular rate that is no greater than about 200 BPM, no greater than about 190 BPM, no greater than about 180 BPM, no greater than about 175 BPM, no greater than about 170 BPM, no greater than about 165 BPM, no greater than about 160 BPM, no greater than about 155 BPM, no greater than about 150 BPM, no greater than about 145 BPM, or no greater than about 140 BPM at initiation of said administering of (b). In some embodiments, administration of said first dose results in a peak plasma

concentration (Cₘₐₓ) of said salt of flecainide in said subject that is at least 200 ng/ml. In some embodiments, said Cₘₐₓ is between about 250 ng/mL and about 1000 ng/ml. In some embodiments, said Cₘₐₓ is between about 300 ng/ml and about 700 ng/ml.

In some embodiments, said second dose comprises said salt of flecainide in an amount that is equivalent to about 40% to about 80% (w/w) of said first dose. In some embodiments, said second dose comprises said salt of flecainide in an amount that is equivalent to about 65% to about 85% (w/w) of said first dose. In some embodiments, said second dose comprises said salt of flecainide in an amount that is equivalent to said first dose. In some embodiments, said second dose comprises said salt of flecainide in an amount that is equivalent to about 15% to about 35% (w/w) of said first dose. In some embodiments, said second dose comprises said salt of flecainide in an amount that is equivalent to about 40% to about 60% (w/w) of said first dose. In some embodiments, said second dose comprises said salt of flecainide in an amount that is equivalent to about 40% (w/w) of said first dose. In some embodiments, said second dose comprises said salt of flecainide in an amount that is equivalent to about 50% (w/w) of said first dose. In some embodiments, said second dose comprises said salt of flecainide in an amount that is equivalent to about 60% (w/w) of said first dose. In some embodiments, said second dose comprises said salt of flecainide in an amount that is equivalent to about 70% (w/w) of said first dose. In some embodiments, said second dose comprises said salt of flecainide in an amount that is equivalent to about 25% (w/w) of said first dose. In some embodiments, said second dose comprises said salt of flecainide in an amount that is equivalent to about 75% (w/w) of said first dose.

In some embodiments, said first dose comprises from about 100 mg to about 250 mg said salt of flecainide. In some embodiments, said first dose comprises from about 100 mg to about 140 mg said salt of flecainide. In some embodiments, said first dose comprises from about 20 mg to about 40 mg said salt of flecainide. In some embodiments, said first dose comprises from about 50 mg to about 70 mg said salt of flecainide. In some embodiments, said first dose comprises from about 80 mg to about 100 mg said salt of flecainide. In some embodiments, said first dose comprises about 120 mg said salt of flecainide. In some embodiments, said first dose comprises about 90 mg said salt of flecainide. In some embodiments, said first dose comprises about 60 mg said salt of flecainide. In some embodiments, said first dose comprises about 30 mg said salt of flecainide.

In some embodiments, said second dose comprises from about 30 mg to about 90 mg said salt of flecainide. In some embodiments, said second dose comprises from about 50 mg to about 70 mg said salt of flecainide. In some embodiments, said second dose comprises from about 20 mg to about 40 mg said salt of flecainide. In some embodiments, said second dose comprises from about 50 mg to about 70 mg said salt of flecainide. In some embodiments, said second dose comprises from about 80 mg to about 100 mg said salt of flecainide.

In some embodiments, administration of said first dose results in a peak plasma concentration (Cₘₐₓ) of said salt of flecainide in said subject that is at least 200 ng/ml. In some embodiments, said Cₘₐₓ is between about 250 ng/ml and about 1000 ng/mL. In some embodiments, said Cₘₐₓ is between about 300 ng/ml and about 700 ng/ml.

The methods, compositions, and kits provided herein can include administration of the pharmaceutical composition via inhalation, e.g., oral or nasal inhalation.

The therapy provided herein can comprise or be suitable for inhalation, e.g., oral or nasal inhalation. In some cases, during administration via oral inhalation, the pharmaceutical agent is inhaled by the patient through the mouth and absorbed by the lungs. In some cases, during administration via nasal inhalation, the pharmaceutical agent is inhaled by the patient through the nose and absorbed by the nasal mucous and/or the lungs.

The inhalation route can avoid first-pass hepatic metabolism, hence dosing variability can be eliminated. Unlike the case for oral tablets or pills, the patient's metabolic rates may not matter as the administration is independent of the metabolic paths experienced when a drug is administered via oral route through gastrointestinal tract, e.g., as tablets, pills, solution, or suspension. A fast onset of action, a potential improvement in efficacy, and/or a reduction in dose can be achieved with the fast absorption of drugs from the nasal mucosa and/or lungs.

The fast absorption rate of drugs through the lungs can be achieved because of the large surface area available in the lungs for aerosols small enough to penetrate central and peripheral lung regions. Consequently, the rate and extent of absorption of drugs delivered via inhalation can yield plasma concentrations vs. time profiles that are comparable with the IV route of administration.

The time for onset of action can be short. For instance, the patient may have normal sinus rhythm within 20 minutes of initiating the administering, such as within 15 minutes, within 10 minutes, or within 5 minutes of initiating the administering. In some cases, the rapid onset of action is advantageous because the longer a patient remains in arrhythmia (i.e. atrial fibrillation), the more difficult it will be to restore normal sinus rhythm.

In some embodiments, the method of the present disclosure allows the patient to avoid other therapies, such as ablation and/or electrical cardioversion. In other embodiments, the method of the present disclosure is used in combination with other therapies, such as before or after electrical cardioversion and/or ablation therapy.

In some aspects of the present disclosure, the compositions or formulations of the pharmaceutical composition via inhalation. The pharmaceutical compositions can be aerosolized prior to administration or can be presented to a user in the form of an aerosol.

The pharmaceutical compositions can be administered using an aerosolization device. The aerosolization device can be a nebulizer, a metered dose inhaler, or a liquid dose instillation device. The aerosolization device can comprise the extrusion of the pharmaceutical preparation through micron or submicron-sized holes with subsequent Rayleigh break-up into fine droplets. The pharmaceutical composition can be delivered by a nebulizer as described in WO 99/16420, by a metered dose inhaler as described in WO 99/16422, by a liquid dose instillation apparatus as described in WO 99/16421, and by a dry powder inhaler as described in U.S. Published Application Nos. 20020017295 and 20040105820, WO 99/16419, WO 02/83220, and U.S. Pat. No. 6,546,929, which are incorporated herein by reference in their entireties. As such, an inhaler can comprise a canister containing the particles or particles and propellant, and wherein the inhaler comprises a metering valve in communication with an interior of the canister. The propellant can be a hydrofluoroalkane.

For instance, the pharmaceutical formulation can be in liquid solution, and can be administered with nebulizers, such as that disclosed in PCT WO 99/16420, the disclosure of which is hereby incorporated in its entirety by reference, in order to provide an aerosolized medicament that can be administered to the pulmonary air passages of a patient in need thereof. Nebulizers known in the art can easily be employed for administration of the claimed formulations. Breath-activated or breath-actuated nebulizers, as well as those comprising other types of improvements which have been, or will be, developed are also compatible with the formulations of the present disclosure and are contemplated as being within the scope thereof.

In some cases, the nebulizer is a breath activated or breath-actuated nebulizer. In some cases, the nebulizer is a hand-held inhaler device (e.g., AeroEclipse® II Breath Actuated Nebulizer (BAN)). In some cases, the nebulizer has a compressed air source. In some cases, the nebulizer converts liquid medication into an aerosol. In some cases, the nebulizer converts liquid medication into an aerosol by extruding the pharmaceutical preparation through micron or submicron-sized holes. In some cases, the nebulizer converts liquid medication into an aerosol so it can be inhaled into the lungs. In some cases, the nebulizer is a small volume nebulizer. In some cases, the nebulizer is a small volume jet nebulizer. In some cases, aerosolized medication is only produced when inhaled through the device. In some cases, the medication is contained in the cup between breaths or during breaks in treatment. In some cases, the medication is contained in the cup until ready to be inhaled.

Nebulizers can impart energy into a liquid pharmaceutical formulation to aerosolize the liquid, and to allow delivery to the pulmonary system, e.g., the lungs, of a patient. A nebulizer comprises a liquid delivery system, such as a container having a reservoir that contains a liquid pharmaceutical formulation. The liquid pharmaceutical formulation generally comprises an active agent that is either in solution or suspended within a liquid medium.

In one type of nebulizer that can be used in the subject methods and kits, generally referred to as a jet nebulizer, compressed gas is forced through an orifice in the container. The compressed gas forces liquid to be withdrawn through a nozzle, and the withdrawn liquid can mix with the flowing gas to form aerosol droplets. A cloud of droplets can then be administered to the patients respiratory tract.

In another type of nebulizer that can be used in the subject methods and kits, generally referred to as a vibrating mesh nebulizer, energy, such as mechanical energy, vibrates a mesh. This vibration of the mesh aerosolizes the liquid pharmaceutical formulation to create an aerosol cloud that is administered to the patient's lungs. In another type of nebulizer that can be used in the subject methods and kits, the nebulizing comprises extrusion through micron or submicron-sized holes followed by Rayleigh break-up into fine droplets.

Alternatively or additionally, the pharmaceutical formulation may be in a liquid form and may be aerosolized using a nebulizer as described in WO 2004/071368, which is herein incorporated by reference in its entirety, as well as U.S. Published application Nos. 2004/0011358 and 2004/0035413, which are both herein incorporated by reference in their entireties. Other examples of nebulizers include, but are not limited to, the Aeroneb®Go or Aeroneb®Pro nebulizers, available from Aerogen Ltd. of Galway, Ireland; the PARI eFlow and other PARI nebulizers available from PARI Respiratory Equipment, Inc. of Midlothian, Va.; the Lumiscope® Nebulizer 6600 or 6610 available from Lumiscope Company, Inc. of East Brunswick, N.J.; and the Omron NE-U22 available from Omron Healthcare, Inc. of Kyoto, Japan. Other examples of nebulizers include devices produced by Medspray (Enschede, The Netherlands).

It has been found that a nebulizer of the vibrating mesh type, such as one that that forms droplets without the use of compressed gas, such as the Aeroneb® Pro provides unexpected improvement in dosing efficiency and consistency. By generating fine droplets by using a vibrating perforated or unperforated membrane, rather than by introducing compressed air, the aerosolized pharmaceutical formulation can be introduced without substantially affecting the flow characteristics. In addition, the generated droplets when using a nebulizer of this type are introduced at a low velocity, thereby decreasing the likelihood of the droplets being driven to an undesired region. It has been found that when using a nebulizer of the extrusion/Rayleigh jet breakup type, the generated droplets are also introduced at a low velocity, thereby decreasing the likelihood of the droplets being driven to an undesired region.

In some cases, the nebulizer that can be used in the subject methods and kits is of the vibrating mesh type. In some cases, the nebulizer that can be used in the subject methods and kits is of the pressurized jet type. In some cases, the nebulizer that can be used in the subject methods and kits is of the extrusion/Rayleigh breakup type. In some cases, the nebulizer is lightweight (at most 60 g, at most 100 g, at most 200 g, at most 250 g) and nearly silent. In some cases, the nebulizer has a sound level less than 35 A-weighted decibels (dBA) at 1 meter. In some cases, the nebulizer has a medication cup capacity of 6 mL. In some cases, the nebulizer has a residual volume of less than 0.3 mL. In some cases, the nebulizer generates an average flow rate of 0.4 mL/min. In some cases, the nebulizer generates an average flow rate of 0.5 mL/min. In some cases, the nebulizer generates an average flow rate of 0.6 mL/min. In some cases, the nebulizer generates an average flow rate of 0.7 mL/min. In some cases, the nebulizer generates an average flow rate of 0.8 mL/min. In some cases, the nebulizer generates an average flow rate of 0.9 mL/min. In some cases, the nebulizer generates an average flow rate of 1.0 mL/min. In some cases, the nebulizer generates an average flow rate of 1.1 mL/min. In some cases, the nebulizer generates an average flow rate of 1.2 mL/min. In some cases, the nebulizer generates an average particle size of 3.0 μm MMAD. In some cases, the nebulizer generates an average particle size between 3.0 μm MMAD and 4.0 μm MMAD. In some cases, the nebulizer generates an average particle size of 3.0 μm MMAD. In some cases, the nebulizer generates an average particle size between 3.0 μm MMAD and 5.0 μm MMAD. In some cases, the nebulizer generates an average particle size of 3.0 μm MMAD. In some cases, the nebulizer generates an average particle size between 3.0 μm MMAD and 6.0 μm MMAD.

In still another type of nebulizer that can be used in the subject methods and kits, ultrasonic waves are generated to directly vibrate and aerosolize the pharmaceutical formulation.

The pharmaceutical formulations disclosed herein can also be administered to the lungs of a patient via aerosolization, such as with a metered dose inhaler. The use of such formulations provides for superior dose reproducibility and improved lung deposition as disclosed in WO 99/16422, hereby incorporated in its entirety by reference. Metered dose inhalers (MDIs) known in the art can be employed for administration of the claimed pharmaceutical compositions. Breath-activated or breath-actuated MDIs and pressurized MDIs (pMDIs), as well as those comprising other types of improvements which have been, or will be, developed are also compatible with the formulations of the present disclosure and, as such, are contemplated as being within the scope thereof.

Along with MDIs and nebulizers, it will be appreciated that the formulations of one or more embodiments of the present disclosure can be used in conjunction with liquid dose instillation or LDI techniques as disclosed in, for example, WO 99/16421, which is incorporated herein by reference in its entirety. Liquid dose instillation involves the direct administration of a formulation to the lung. With respect to LDI the formulations are preferably used in conjunction with partial liquid ventilation or total liquid ventilation. Moreover, one or more embodiments of the present disclosure may further comprise introducing a therapeutically beneficial amount of a physiologically acceptable gas (such as nitric oxide or oxygen) into the pharmaceutical microdispersion prior to, during or following administration.

The pharmaceutical composition of one or more embodiments of the present disclosure can have improved emitted dose efficiency. Accordingly, high doses of the pharmaceutical composition can be delivered using a variety of aerosolization devices and techniques.

The emitted dose (ED) of the particles of the present disclosure may be greater than about 30%, such as greater than about 40%, greater than about 50%, greater than about 60%, or greater than about 70%.

The pharmaceutical composition can be administered to the patient on an as-needed basis. For instance, the methods, kits, and compositions can find particular use in treating a subject experiencing a heart condition, e.g., cardiac arrhythmia, e.g., atrial arrhythmia. In some cases, a subject is administered with the therapy described herein when he/she is experiencing atrial arrhythmia. In some cases, the pharmaceutical composition is administered to a subject after the onset of an episode of cardiac arrhythmia. In other cases, the subject is treated between episodes of cardiac arrhythmias.

The dose of the antiarrhythmic agent, e.g., flecainide salt, e.g., flecainide acetate, can be administered during a single inhalation or can be administered during several inhalations. The fluctuations of antiarrhythmic pharmaceutical agent concentration can be reduced by administering the pharmaceutical composition more often or can be increased by administering the pharmaceutical composition less often. Therefore, the pharmaceutical composition provided herein can be administered from about four times daily to about once a month, such as about once daily to about once every two weeks, about once every two days to about once a week, and about once per week.

In some cases, the antiarrhythmic pharmaceutical agent is delivered over two or more inhalations. In some cases, time between the two or more inhalations is from about 0.1 to 10 minutes. The antiarrhythmic pharmaceutical agent is administered in the described dose in less than 60 minutes, less than 50 minutes, less than 40 minutes, less than 30 minutes, less than 20 minutes, less than 15 minutes, less than 10 minutes, less than 7 minutes, less than 5 minutes, in less than 3 minutes, in less than 2 minutes, or in less than 1 minute. In some cases, delivery of the required dose of antiarrhythmic pharmaceutical agent is completed with 1, 2, 3, 4, 5, or 6 inhalations. In some cases, each inhalation is performed for about 0.5, 1, 1.2, 1.5, 1.8, 2, 2.2, 2.5, 2.8, 3, 3.2, 3.5, 3.8, 4, 4.2, 4.5, 4.8, or 5 minutes. In some cases, each inhalation is performed for longer than 5 minutes. In some cases, each inhalation is performed for up to 4.5 minutes. In some cases, each inhalation comprises at least 60 inhalation breaths, 50 inhalation breaths, 40 inhalation breaths, 30 inhalation breaths, 20 inhalation breaths, 10 inhalation breaths, 8 inhalation breaths, 6 inhalation breaths, 4 inhalation breaths, 3 inhalation breaths, 2 inhalation breaths or 1 inhalation breath. In some cases, each inhalation comprises no more than 100 inhalation breaths, 90 inhalation breaths, 80 inhalation breaths, 70 inhalation breaths, 60 inhalation breaths, 50 inhalation breaths, 40 inhalation breaths, 30 inhalation breaths, or 20 inhalation breaths. In some cases, inhalation of the antiarrhythmic pharmaceutical agent is performed with deep lung breath that lasts for longer than 1 second, 2 seconds, 3 seconds, or 4 seconds. In some cases, inhalation of the antiarrhythmic pharmaceutical agent is performed with deep lung breath that lasts for about 1 second, 2 seconds, 3 seconds, or 4 seconds.

In some embodiments, during inhalational delivery of the antiarrhythmic pharmaceutical agent, the subject takes, or is instructed to take, a break between two inhalations. In such embodiments, the break between two inhalations lasts for about 0.1 to 10 minutes, such as, 0.2 to 5, 1 to 5, 1.5 to 5, 2 to 5, 3 to 5, 4 to 5, 1 to 1.5, 1 to 2, 1 to 2.5, 1 to 3, 1 to 3.5, 1 to 4, 1.5 to 2, 1.5 to 2.5, or 1.5 to 3 minutes. In some cases, the subject takes, or is instructed to take, a break for about 1 minute between two inhalations. In some cases, the inhalation pattern for delivery of a single dose goes as follows: a first inhalation for about 4 to 4.5 minutes, a break for about 1 minute, and a second inhalation for about 4 to 4.5 minutes; a first inhalation for about 4 to 4.5 minutes, a break for about 30 seconds, and a second inhalation for about 4 to 4.5 minutes; a first inhalation for about 4 to 4.5 minutes, a first break for about 1 minute, and a second inhalation for about 4 to 4.5 minutes; a second break for about 1 minutes, and a third inhalation for about 4 to 4.5 minutes; or a first inhalation for about 4 to 4.5 minutes, a first break for about 30 seconds, and a second inhalation for about 4 to 4.5 minutes; a second break for about 30 seconds, and a third inhalation for about 4 to 4.5 minutes.

In some embodiments, the subject inhales, or is instructed to inhale an aerosolized pharmaceutical composition of the disclosure via tidal breathing for a certain duration of time. In some embodiments, after inhaling the pharmaceutical composition for a first duration of time (e.g., about 3.5 minutes), the subject pauses inhalation of the aerosolized pharmaceutical composition for another duration of time, such as a period equivalent to a break as specified above (e.g., about 1 minute), and then subsequently resumes inhaling the aerosolized pharmaceutical composition for another duration of time (e.g., about 3.5 minutes). In some embodiments, the subject inhales the aerosolized pharmaceutical composition for 3.5 minutes, pauses inhalation of the aerosolized pharmaceutical composition for about 1 minute, and then resumes inhaling the aerosolized pharmaceutical composition for a subsequent 3.5 minutes.

In some embodiments, the subject inhales an aerosolized pharmaceutical composition of the disclosure via tidal breathing. As used herein, “tidal breathing” can refer to inhalation and exhalation during restful breathing. For example, tidal breathing can include inhaling the aerosolized pharmaceutical composition while breathing at a rate of 10 to 14 breaths a minute.

In one version, the antiarrhythmic can be administered daily. In this case, the daily dosage of the flecainide acetate ranges from about 0.1 mg to about 600 mg, such as about 0.5 mg to about 500 mg, about 1 mg to about 400 mg, about 2 mg to about 300 mg, and about 3 mg to about 200 mg.

In some cases, the therapy provided herein is provided to a subject for more than once on an as-needed basis. For instance, the present disclosure may involve a follow-up inhalation if no cardioversion occurs after an initial inhalation. In some instances, if no cardioversion occurs within 30 minutes of the initial inhalation, the follow-up dosage is higher or the same as the initial dosage.

The dosing can be guided by how the patient feels. Additionally or alternatively, dosing can be guided by using a portable/mobile ECG device. For instance, the dosing may be guided by using a Holter monitor.

In another version, the pharmaceutical composition is administered prophylactically to a subject who is likely to develop an arrhythmia. For example, a patient who has a history of arrhythmias can be prophylactically treated with a pharmaceutical composition comprising antiarrhythmic pharmaceutical agent to reduce the likelihood of developing an arrhythmia.

The pharmaceutical composition can be administered to a patient in any regimen which is effective to prevent an arrhythmia. Illustrative prophylactic regimes include administering an antiarrhythmic pharmaceutical agent as described herein 1 to 21 times per week.

In some cases, patient receiving administration of pharmaceutical composition according to the method described herein needs to meet one or more of the following ECG criteria: P waves not seen on the ECG; fibrillatory waves are coarse; there are varying RR intervals; there are irregular-irregular QRS complexes; or there is an elevated ventricular rate. In some cases, method of treatment disclosed herein comprises confirming a patient in atrial fibrillation episode via ECG. In some cases, the method comprises confirming a patient having the following ECG features before administering the pharmaceutical composition as described herein: P waves not seen on the ECG; fibrillatory waves are coarse; there are varying RR intervals; there are irregular-irregular QRS complexes; and there is an elevated ventricular rate. In some cases, a patient receiving administration of pharmaceutical composition according to the method described herein has a medical history that is current within predetermined time period and qualifies the patient to receive flecainide safely. In some cases, the patient's current AF episode has had a duration of less than 48 hours. In some cases, total flecainide exposure the patient receives within past 24 hour period does not exceed 320 mg.

The amount of the flecainide salt that is delivered to the subject (e.g., approximately the amount of the flecainide salt exiting a mouthpiece when being inhaled by the subject) for the treatment of arrhythmia, e.g., atrial arrhythmia, e.g., atrial fibrillation, can be from about 50 mg to about 300 mg, such as 50 mg to 60 mg, 50 mg to 70 mg, 50 mg to 80 mg, 50 mg to 90 mg, 50 mg to 100 mg, 50 mg to 110 mg, 50 mg to 120 mg, 50 mg to 130 mg, 50 mg to 140 mg, 50 mg to 150 mg, 50 mg to 160 mg, 50 mg to 170 mg, 50 mg to 180 mg, 50 mg to 190 mg, 50 mg to 200 mg, 50 mg to 210 mg, 50 mg to 220 mg, 50 mg to 230 mg, 50 mg to 240 mg, 50 mg to 250 mg, 50 mg to 260 mg, 50 mg to 270 mg, 50 mg to 280 mg, 50 mg to 290 mg, 70 mg to 80 mg, 70 mg to 90 mg, 70 mg to 100 mg, 70 mg to 110 mg, 70 mg to 120 mg, 70 mg to 130 mg, 70 mg to 140 mg, 70 mg to 170 mg, 70 mg to 160 mg, 70 mg to 170 mg, 70 mg to 180 mg, 70 mg to 190 mg, 70 mg to 200 mg, 70 mg to 210 mg, 70 mg to 220 mg, 70 mg to 230 mg, 70 mg to 240 mg, 70 mg to 270 mg, 70 mg to 260 mg, 70 mg to 270 mg, 70 mg to 280 mg, 70 mg to 290 mg, 70 mg to 300 mg, 80 mg to 90 mg, 80 mg to 100 mg, 80 mg to 110 mg, 80 mg to 120 mg, 80 mg to 130 mg, 80 mg to 140 mg, 80 mg to 150 mg, 80 mg to 160 mg, 80 mg to 170 mg, 80 mg to 180 mg, 80 mg to 190 mg, 80 mg to 200 mg, 80 mg to 210 mg, 80 mg to 220 mg, 80 mg to 230 mg, 80 mg to 240 mg, 80 mg to 250 mg, 80 mg to 260 mg, 80 mg to 270 mg, 80 mg to 280 mg, 80 mg to 290 mg, 80 mg to 300 mg, 100 mg to 110 mg, 100 mg to 120 mg, 100 mg to 130 mg, 100 mg to 140 mg, 100 mg to 150 mg, 100 mg to 160 mg, 100 mg to 170 mg, 100 mg to 180 mg, 100 mg to 190 mg, 100 mg to 200 mg, 100 mg to 210 mg, 100 mg to 220 mg, 100 mg to 230 mg, 100 mg to 240 mg, 100 mg to 250 mg, 100 mg to 260 mg, 100 mg to 270 mg, 100 mg to 280 mg, 100 mg to 290 mg, 100 mg to 300 mg, 120 mg to 140 mg, 120 mg to 150 mg, 120 mg to 160 mg, 120 mg to 170 mg, 120 mg to 180 mg, 120 mg to 190 mg, 120 mg to 200 mg, 120 mg to 210 mg, 120 mg to 220 mg, 120 mg to 230 mg, 120 mg to 240 mg, 120 mg to 250 mg, 120 mg to 260 mg, 120 mg to 270 mg, 120 mg to 280 mg, 120 mg to 290 mg, 120 mg to 300 mg, 150 mg to 160 mg, 150 mg to 170 mg, 150 mg to 180 mg, 150 mg to 190 mg, 150 mg to 200 mg, 150 mg to 210 mg, 150 mg to 220 mg, 150 mg to 230 mg, 150 mg to 240 mg, 150 mg to 250 mg, 150 mg to 260 mg, 150 mg to 270 mg, 150 mg to 280 mg, 150 mg to 290 mg, 150 mg to 300 mg, 180 mg to 200 mg, 180 mg to 210 mg, 180 mg to 220 mg, 180 mg to 230 mg, 180 mg to 240 mg, 180 mg to 250 mg, 180 mg to 260 mg, 180 mg to 270 mg, 180 mg to 280 mg, 180 mg to 290 mg, 180 mg to 300 mg, 200 mg to 220 mg, 200 mg to 230 mg, 200 mg to 240 mg, 200 mg to 250 mg, 200 mg to 260 mg, 200 mg to 270 mg, 200 mg to 280 mg, 200 mg to 290 mg, 200 mg to 300 mg, 220 mg to 240 mg, 220 mg to 250 mg, 220 mg to 260 mg, 220 mg to 270 mg, 220 mg to 280 mg, 220 mg to 290 mg, 220 mg to 300 mg, 250 mg to 260 mg, 250 mg to 270 mg, 250 mg to 280 mg, 250 mg to 290 mg, 250 mg to 300 mg, 280 mg to 260 mg, 280 mg to 270 mg, 280 mg to 280 mg, 280 mg to 290 mg, or 280 mg to 300 mg.

In one version, the amount of the flecainide salt that is delivered to the subject (e.g., approximately the amount of the flecainide salt exiting the aerosolization device when being inhaled by the subject) for the treatment of arrhythmia, e.g., atrial arrhythmia, e.g., atrial fibrillation, is at least about 50 mg, at least about 60 mg, at least about 70 mg, at least about 80 mg, at least about 90 mg, at least about 100 mg, at least about 110 mg, at least about 120 mg, at least about 130 mg, at least about 140 mg, at least about 150 mg, at least about 160 mg, at least about 170 mg, at least about 180 mg, at least about 190 mg, at least about 200 mg, at least about 210 mg, at least about 220 mg, at least about 230 mg, at least about 240 mg, at least about 250 mg, at least about 260 mg, at least about 270 mg, at least about 280 mg, or at least about 290 mg.

In one version, the amount of the flecainide salt that is delivered to the subject (e.g., approximately the amount of the flecainide salt exiting a mouthpiece when being inhaled by the subject) for the treatment of arrhythmia, e.g., atrial arrhythmia, e.g., atrial fibrillation, is at most about 100 mg, at most about 110 mg, at most about 120 mg, at most about 130 mg, at most about 140 mg, at most about 150 mg, at most about 160 mg, at most about 170 mg, at most about 180 mg, at most about 190 mg, at most about 200 mg, at most about 210 mg, at most about 220 mg, at most about 230 mg, at most about 240 mg, at most about 250 mg, at most about 260 mg, at most about 270 mg, at most about 280 mg, or at most about 290 mg.

In some cases, the amount of the flecainide salt that is delivered to the subject (e.g., approximately the amount of the flecainide salt exiting a mouthpiece when being inhaled by the subject) for the treatment of arrhythmia, e.g., atrial arrhythmia, e.g., atrial fibrillation, is about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, or about 290 mg.

There can be loss of a pharmaceutical composition along the respiratory pathway when the pharmaceutical composition is inhaled by a subject, so that not all the pharmaceutical composition reaches the lung for absorption into the systemic circulation. In some embodiments, the estimated total lung dose (eTLD, e.g., a theoretical value that measures the dose of the pharmaceutical active ingredient that reaches the lung, e.g., about 70% of the dose exiting the aerosolization device) of the flecainide acetate that is delivered according to the methods provided herein is from about 40 mg to about 180 mg, such as 40 mg to 60 mg, 40 mg to 70 mg, 40 mg to 80 mg, 40 mg to 90 mg, 40 mg to 100 mg, 40 mg to 110 mg, 40 mg to 120 mg, 40 mg to 130 mg, 40 mg to 140 mg, 40 mg to 150 mg, 40 mg to 160 mg, 40 mg to 170 mg, 40 mg to 180 mg, 50 mg to 60 mg, 50 mg to 70 mg, 50 mg to 80 mg, 50 mg to 90 mg, 50 mg to 100 mg, 50 mg to 110 mg, 50 mg to 120 mg, 50 mg to 130 mg, 50 mg to 140 mg, 50 mg to 150 mg, 50 mg to 160 mg, 50 mg to 170 mg, 50 mg to 180 mg, 70 mg to 80 mg, 70 mg to 90 mg, 70 mg to 100 mg, 70 mg to 110 mg, 70 mg to 120 mg, 70 mg to 130 mg, 70 mg to 140 mg, 70 mg to 170 mg, 70 mg to 160 mg, 70 mg to 170 mg, 70 mg to 180 mg, 80 mg to 90 mg, 80 mg to 100 mg, 80 mg to 110 mg, 80 mg to 120 mg, 80 mg to 130 mg, 80 mg to 140 mg, 80 mg to 150 mg, 80 mg to 160 mg, 80 mg to 170 mg, 80 mg to 180 mg, 90 mg to 100 mg, 90 mg to 110 mg, 90 mg to 120 mg, 90 mg to 130 mg, 90 mg to 140 mg, 90 mg to 150 mg, 90 mg to 160 mg, 90 mg to 170 mg, 90 mg to 180 mg, 100 mg to 110 mg, 100 mg to 120 mg, 100 mg to 130 mg, 100 mg to 140 mg, 100 mg to 150 mg, 100 mg to 160 mg, 100 mg to 170 mg, 100 mg to 180 mg, 120 mg to 140 mg, 120 mg to 150 mg, 120 mg to 160 mg, 120 mg to 170 mg, 120 mg to 180 mg, 150 mg to 160 mg, 150 mg to 170 mg, or 150 mg to 180 mg.

In some cases, the eTLD of the flecainide acetate that is delivered according to the methods provided herein is at least about 40 mg, at least about 60 mg, at least about 70 mg, at least about 80 mg, at least about 90 mg, at least about 100 mg, at least about 110 mg, at least about 120 mg, at least about 130 mg, at least about 140 mg, at least about 150 mg, at least about 160 mg, or at least about 170 mg. In some cases, the eTLD of the flecainide acetate that is delivered according to the methods provided herein is at most about 60 mg, at most about 70 mg, at most about 80 mg, at most about 90 mg, at most about 100 mg, at most about 110 mg, at most about 120 mg, at most about 130 mg, at most about 140 mg, at most about 150 mg, at most about 160 mg, at most about 170 mg, or at most about 180 mg. In some cases, the eTLD of the flecainide acetate that is delivered according to the methods provided herein is about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 160 mg, about 170 mg, or about 180 mg.

This method of treatment results in a pulsatile pharmacokinetic profile and transient pharmacodynamic effect mimicking the effect of an IV. This method delivers high drug concentrations that are safe and effective to the heart, while the distribution to the rest of the body results in the drug being diluted to sub-therapeutic levels. This method is the shortest route of delivery to the heart next to intra-cardial injection. This provides the convenience of self-administration like the “pill-in-the-pocket” approach, but the effectiveness and fast onset of action of an IV. Although the delivery of medications through the lung for systemic effect is not new, it was thought it wouldn't be effective to the heart, because of the fast passage of drug through it. The animal and human PK/PD data in this study show that the drug exposure is sufficient for therapeutic effect at a much lower dose compared to other routes of administration. This method ensures dug concentrations in overall plasma are much lower than what is achieved by oral/IV hence minimizing drug-drug interactions and side effects.

In some cases, the Tₘₐₓ of the antiarrhythmic pharmaceutical agent administered via inhalation can be within about 30 min, such as within about 0.5, 1, 1.5, 2, 2.5, 3, 3.5, 4, 4.5, 5, 6, 8, 10, 15, 20, 25, or 30 minutes. In some cases, the Tₘₐₓ of the antiarrhythmic pharmaceutical agent administered via inhalation is within about 5 minutes. In some cases, the Tₘₐₓ of the antiarrhythmic pharmaceutical agent administered via inhalation can be from about 0.1 minute to about 30 minutes, such as 0.1-0.5, 0.1-1, 0.1-1.5, 0.1-2, 0.1-2.5, 0.1-3, 0.1-3.5, 0.1-4, 0.1-4.5, 0.1-5, 0.1-6, 0.1-8, 0.1-10, 0.1-15, 0.1-20, 0.1-25, 0.1-30, 0.2-0.5, 0.2-1, 0.2-1.5, 0.2-2, 0.2-2.5, 0.2-3, 0.2-3.5, 0.2-4, 0.2-4.5, 0.2-5, 0.2-6, 0.2-8, 0.2-10, 0.2-15, 0.2-20, 0.2-25, 0.2-30, 0.3-0.5, 0.3-1, 0.3-1.5, 0.3-2, 0.3-2.5, 0.3-3, 0.3-3.5, 0.3-4, 0.3-4.5, 0.3-5, 0.3-6, 0.3-8, 0.3-10, 0.3-15, 0.3-20, 0.3-25, 0.3-30, 0.5-1, 0.5-1.5, 0.5-2, 0.5-2.5, 0.5-3, 0.5-3.5, 0.5-4, 0.5-4.5, 0.5-5, 0.5-6, 0.5-8, 0.5-10, 0.5-15, 0.5-20, 0.5-25, 0.5-30, 1-1.5, 1-2, 1-2.5, 1-3, 1-3.5, 1-4, 1-4.5, 1-5, 1-6, 1-8, 1-10, 1-15, 1-20, 1-25, 1-30, 1.5-2, 1.5-2.5, 1.5-3, 1.5-3.5, 1.5-4, 1.5-4.5, 1.5-5, 1.5-6, 1.5-8, 1.5-10, 1.5-15, 1.5-20, 1.5-25, 1.5-30, 2-2.5, 2-3, 2-3.5, 2-4, 2-4.5, 2-5, 2-6, 2-8, 2-10, 2-15, 2-20, 2-25, 2-30, 2.5-3, 2.5-3.5, 2.5-4, 2.5-4.5, 2.5-5, 2.5-6, 2.5-8, 2.5-10, 2.5-15, 2.5-20, 2.5-25, 2.5-30, 3-3.5, 3-4, 3-4.5, 3-5, 3-6, 3-8, 3-10, 3-15, 3-20, 3-25, 3-30, 3.5-4, 3.5-4.5, 3.5-5, 3.5-6, 3.5-8, 3.5-10, 3.5-15, 3.5-20, 3.5-25, 3.5-30, 4-4.5, 4-5, 4-6, 4-8, 4-10, 4-15, 4-20, 4-25, 4-30, 4.5-5, 4.5-6, 4.5-8, 4.5-10, 4.5-15, 4.5-20, 4.5-25, 4.5-30, 5-6, 5-8, 5-10, 5-15, 5-20, 5-25, 5-30, 5.5-6, 5.5-8, 5.5-10, 5.5-15, 5.5-20, 5.5-25, 5.5-30, 6-8, 6-10, 6-15, 6-20, 6-25, 6-30, 8-10, 8-15, 8-20, 8-25, 8-30, 10-15, 10-20, 10-25, 10-30, 15-20, 15-25, 15-30, 20-25, 20-30, or 25-30 min. A range given out in the present disclosure can be a range between two accurate numerical values, in some cases, a range in the present disclosure can also refer to a range between two approximate numerical values. For instance, “1-10” can refer to “from 1 to 10” in some cases, while in other case, “1-10” can refer to “from about 1 to about 10”. In some cases, the Tₘₐₓ of the antiarrhythmic pharmaceutical agent administered via inhalation can be 0.01-5, 0.02-5, 0.03-5, 0.04-5, 0.05-5, 0.06-5, 0.07-5, 0.08-5, 0.09-5, 0.12-5, 0.14-5, 0.15-5, 0.16-5, 0.18-5, 0.2-5, 0.24-5, 0.26-5, 0.28-5, 0.3-5, 0.35-5, 0.4-5, 0.5-5, 0.6-5, 0.7-5, 0.8-5, 0.9-5, or 1-5 min. In some cases, the Tₘₐₓ of the antiarrhythmic pharmaceutical agent administered via inhalation can be from about 0.1 to about 3 min. In some cases, the Tₘₐₓ of the antiarrhythmic pharmaceutical agent administered via inhalation can be from about 0.1 to about 5 min. In some cases, the Tₘₐₓ of the antiarrhythmic pharmaceutical agent (e.g., flecainide) administered via inhalation can be from about 0.2 to about 5 min. In one or more embodiments, the antiarrhythmic pharmaceutical agent is a class I, class II, class III, or class IV antiarrhythmic. In some embodiments, the antiarrhythmic pharmaceutical agent is a class Ic, antiarrhythmic. In other embodiments, the antiarrhythmic pharmaceutical agent is flecainide or a pharmaceutically acceptable salt thereof.

In some cases, the Tₘₐₓ is calculated as the amount of time at which the maximum plasma concentration of the antiarrhythmic pharmaceutical agent is observed. In some cases, the Tₘₐₓ can be calculated as the amount of time after administration of the antiarrhythmic pharmaceutical agent when the maximum plasma concentration is reached. In some cases, the Tₘₐₓ can be calculated as the amount of time after the initiation of the administration of the antiarrhythmic pharmaceutical agent when the maximum plasma concentration is reached. In some cases, the Tₘₐₓ can be calculated as the amount of time after the completion of the administration of the antiarrhythmic pharmaceutical agent when the maximum plasma concentration is reached. In some cases, the Tₘₐₓ can be calculated from plasma concentration of the antiarrhythmic pharmaceutical agent measured in the left ventricular chamber. In some cases, the Tₘₐₓ can be calculated from plasma concentration of the antiarrhythmic pharmaceutical agent measured in the pulmonary artery. In some cases, the Tₘₐₓ can be calculated from plasma concentration of the antiarrhythmic pharmaceutical agent measured in the vein (e.g., femoral vein). In some cases, the Tₘₐₓ can be measured in a human PK/PD study.

The term “human PK/PD study” as used herein can refer to any settings where a human subject receives administration of a single dose of the antiarrhythmic agent as provided herein and a pharmacokinetic (PK) or pharmacodynamic (PD) parameter is measured from the human subject after the administration of the antiarrhythmic agent. In some cases, a human PK/PD study as provided herein can refer to a clinical study performed in a clinic or hospital settings. In some cases, the human PK/PD study can be a single center or multi-center study. A human PK/PD study can be performed on healthy human subjects or human cardiovascular patients. In some cases, the patients with cardiovascular disease experience arrhythmia as described herein. In some cases, a human PK/PD study can be a single-dose study, in other cases, a human PK/PD study can be a multi-dose (e.g. escalating doses) study.

Pharmacokinetics (PK) as described herein is concerned with the time course of a therapeutic agent, such as an antiarrhythmic pharmaceutical agent, e.g., flecainide, in the body. Pharmacodynamics (PD) is concerned with the relationship between pharmacokinetics and efficacy in vivo. PK/PD parameters correlate the therapeutic agent, such as an antiarrhythmic pharmaceutical agent, e.g., flecainide, with efficacious activity.

Any standard pharmacokinetic protocol can be used in a human PK/PD study to determine blood plasma concentration profile in humans following administration of a formulation described herein, such as an inhalable formulation comprising flecainide, and thereby establish whether that formulation meets the pharmacokinetic criteria set out herein. For example, but in no way limiting, a type of a randomized single-dose crossover study can be utilized using a group of healthy adult human subjects. The number of subjects can be sufficient to provide adequate control of variation in a statistical analysis, and is typically about 8 or greater, e.g., about 10, 12, 14, 16, 18, 20, or 25. In certain embodiments a smaller group can be used. In one embodiment, a subject receives administration, at time zero, a single dose of an inhalable formulation described herein, e.g., an inhalable formulation comprising flecainide. Blood samples are collected from each subject prior to administration and at several intervals after administration. Plasma can be separated from the blood samples by centrifugation and the separated plasma is analyzed, for example, by a validated high performance liquid chromatography/tandem weight spectrometry (LC/APCI-MS/MS) procedure such as, for example, those described in Ramu et al., Journal of Chromatography B, 751:49-59 (2001). In other embodiments, data from a single subject may be collected and may be used to construct a PK profile and may be indicative of an enhanced pharmacokinetic profile.

In some cases, the Cₘₐₓ of the antiarrhythmic pharmaceutical agent administered via inhalation can be from about 10 ng/mL to about 5000 ng/mL, such as from about 10-30, 10-50, 10-70, 10-80, 10-90, 10-100, 10-110, 10-120, 10-130, 10-140, 10-150, 10-160, 10-170, 10-180, 10-190, 10-200, 10-250, 10-300, 10-350, 10-400, 10-450, 10-500, 10-550, 10-600, 10-650, 10-700, 10-800, 10-900, 10-1000, 10-1500, 10-2000, 10-3000, 10-4000, 10-5000, 20-30, 20-50, 20-70, 20-80, 20-90, 20-100, 20-110, 20-120, 20-130, 20-140, 20-150, 20-160, 20-170, 20-180, 20-190, 20-200, 20-250, 20-300, 20-350, 20-400, 20-450, 20-500, 20-550, 20-600, 20-650, 20-700, 20-800, 20-900, 20-1000, 20-1500, 20-2000, 20-3000, 20-4000, 20-5000, 30-50, 30-70, 30-80, 30-90, 30-100, 30-110, 30-120, 30-130, 30-140, 30-150, 30-160, 30-170, 30-180, 30-190, 30-200, 30-250, 30-300, 30-350, 30-400, 30-450, 30-500, 30-550, 30-600, 30-650, 30-700, 30-800, 30-900, 30-1000, 30-1500, 30-2000, 30-3000, 30-4000, 30-5000, 50-70, 50-80, 50-90, 50-100, 50-110, 50-120, 50-130, 50-140, 50-150, 50-160, 50-170, 50-180, 50-190, 50-200, 50-250, 50-300, 50-350, 50-400, 50-450, 50-500, 50-550, 50-600, 50-650, 50-700, 50-800, 50-900, 50-1000, 50-1500, 50-2000, 50-3000, 50-4000, 50-5000, 70-80, 70-90, 70-100, 70-110, 70-120, 70-130, 70-140, 70-150, 70-160, 70-170, 70-180, 70-190, 70-200, 70-250, 70-300, 70-350, 70-400, 70-450, 70-500, 70-550, 70-600, 70-650, 70-700, 70-800, 70-900, 70-1000, 70-1500, 70-2000, 70-3000, 350-4000, 350-5000, 400-450, 400-500, 400-550, 400-600, 400-650, 400-700, 400-800, 400-900, 400-1000, 400-1500, 400-2000, 400-3000, 400-4000, 400-5000, 450-500, 450-550, 450-600, 450-650, 450-700, 450-800, 450-900, 450-1000, 450-1500, 450-2000, 450-3000, 450-4000, 450-5000, 500-550, 500-600, 500-650, 500-700, 500-800, 500-900, 500-1000, 500-1500, 500-2000, 500-3000, 500-4000, 500-5000, 550-600, 550-650, 550-700, 550-800, 550-900, 550-1000, 550-1500, 550-2000, 550-3000, 550-4000, 550-5000, 600-650, 600-700, 600-800, 600-900, 600-1000, 600-1500, 600-2000, 600-3000, 600-4000, 600-5000, 650-700, 650-800, 650-900, 650-1000, 650-1500, 650-2000, 650-3000, 650-4000, 650-5000, 700-800, 700-900, 700-1000, 700-1500, 700-2000, 700-3000, 700-4000, 700-5000, 800-900, 800-1000, 800-1500, 800-2000, 800-3000, 800-4000, 800-5000, 900-1000, 900-1500, 900-2000, 900-3000, 900-4000, 900-5000, 1000-1500, 1000-2000, 1000-3000, 1000-4000, 1000-5000, 1500-2000, 1500-3000, 1500-4000, 1500-5000, 2000-3000, 2000-4000, 2000-5000, 3000-4000, 3000-5000, or 4000-5000 ng/mL. In some cases, the Cₘₐₓ of the antiarrhythmic pharmaceutical agent administered via inhalation can be from about 20 ng/mL to about 500 ng/mL, such as 20-500, 30-500, 40-500, 50-500, 60-500, 70-500, 80-500, 90-500, 100-500, 150-500, 200-500, or 250-500 ng/ml. In some cases, the Cₘₐₓ of the antiarrhythmic pharmaceutical agent administered via inhalation can be from about 50 to about 500 ng/mL. In some cases, the Cₘₐₓ of the antiarrhythmic pharmaceutical agent administered via inhalation can be from about 200 to about 500 ng/ml. In some cases, the Cₘₐₓ of the antiarrhythmic pharmaceutical agent administered via inhalation is at least about 200 ng/ml. In some cases, the Cₘₐₓ of the antiarrhythmic pharmaceutical agent administered via inhalation is at least about 250 ng/mL. In one or more embodiments antiarrhythmic pharmaceutical agent is a class I, class II, class III, or class IV antiarrhythmic. In some embodiments, the antiarrhythmic pharmaceutical agent is a class Ic, antiarrhythmic. In other embodiments, the antiarrhythmic pharmaceutical agent is flecainide or a pharmaceutically acceptable salt thereof.

In some cases, the Cₘₐₓ can be calculated as the maximum plasma concentration of the antiarrhythmic pharmaceutical agent observed. In some cases, the Cₘₐₓ can be calculated as the peak plasma concentration that the antiarrhythmic pharmaceutical agent achieves after the drug has been administrated. In some cases, the Cₘₐₓ can be calculated from plasma concentration of the antiarrhythmic pharmaceutical agent measured in the left ventricular chamber. In some cases, the Cₘₐₓ can be calculated from plasma concentration of the antiarrhythmic pharmaceutical agent measured in the pulmonary artery. In some cases, the Cₘₐₓ can be calculated from plasma concentration of the antiarrhythmic pharmaceutical agent measured in the vein (e.g., femoral vein). In some cases, the Cₘₐₓ can be measured in a human PK/PD study.

In some cases, the AUC_{Last} of the antiarrhythmic pharmaceutical agent administered via inhalation can be from about 100 hr*ng/ml to about 10000 hr*ng/mL, such as from 100-200, 100-300, 100-400, 100-420, 100-440, 100-460, 100-480, 100-500, 100-520, 100-540, 100-560, 100-580, 100-600, 100-620, 100-640, 100-660, 100-680, 100-700, 100-800, 100-900, 100-1000, 100-1500, 100-2000, 100-3000, 100-3500, 100-4000, 100-4500, 100-5000, 100-5500, 100-6000, 100-6500, 100-7000, 100-8000, 100-9000, 100-10000, 200-300, 200-400, 200-420, 200-440, 200-460, 200-480, 200-500, 200-520, 200-540, 200-560, 200-580, 200-600, 200-620, 200-640, 200-660, 200-680, 200-700, 200-800, 200-900, 200-1000, 200-1500, 200-2000, 200-3000, 200-3500, 200-4000, 200-4500, 200-5000, 200-5500, 200-6000, 200-6500, 200-7000, 200-8000, 200-9000, 200-10000, 300-400, 300-420, 300-440, 300-460, 300-480, 300-500, 300-520, 300-540, 300-560, 300-580, 300-600, 300-620, 300-640, 300-660, 300-680, 300-700, 300-800, 300-900, 300-1000, 300-1500, 300-2000, 300-3000, 300-3500, 300-4000, 300-4500, 300-5000, 300-5500, 300-6000, 300-6500, 300-7000, 300-8000, 300-9000, 300-10000, 400-420, 400-440, 400-460, 400-480, 400-500, 400-520, 400-540, 400-560, 400-580, 400-600, 400-620, 400-640, 400-660, 400-680, 400-700, 400-800, 400-900, 400-1000, 400-1500, 400-2000, 400-3000, 400-3500, 400-4000, 400-4500, 400-5000, 400-5500, 400-6000, 400-6500, 400-7000, 400-8000, 400-9000, 400-10000, 420-440, 420-460, 420-480, 420-500, 420-520, 420-540, 420-560, 420-580, 420-600, 420-620, 420-640, 420-660, 420-680, 420-700, 420-800, 420-900, 420-1000, 420-1500, 420-2000, 420-3000, 420-3500, 420-4000, 5500-9000, 5500-10000, 6000-6500, 6000-7000, 6000-8000, 6000-9000, 6000-10000, 6500-7000, 6500-8000, 6500-9000, 6500-10000, 7000-8000, 7000-9000, 7000-10000, 8000-9000, 8000-10000, or 9000-10000 hr*ng/mL. In some cases, the AUC_{Last} of the antiarrhythmic pharmaceutical agent administered via inhalation can be from about 200 to about 2000 hr*ng/mL. In some cases, the AUC_{Last} of the antiarrhythmic pharmaceutical agent administered via inhalation can be from about 500 to about 800 hr*ng/mL. In some cases, the AUC_{Last} of the antiarrhythmic pharmaceutical agent administered via inhalation can be from about 400 to about 600 hr*ng/mL. In one or more embodiments antiarrhythmic pharmaceutical agent is a class I, class II, class III, or class IV antiarrhythmic. In some embodiments, the antiarrhythmic pharmaceutical agent is a class Ic, antiarrhythmic. In other embodiments, the antiarrhythmic pharmaceutical agent is flecainide or a pharmaceutically acceptable salt thereof.

In some cases, the AUC_{Last} can be calculated as the area under the concentration-time curve up to the last measurable concentration. In some cases, the AUC_{Last} can be calculated as the total drug exposure over time. In some cases, the AUC_{Last} can be calculated from plasma concentration of the antiarrhythmic pharmaceutical agent measured in the left ventricular chamber. In some cases, the AUC_{Last} can be calculated from plasma concentration of the antiarrhythmic pharmaceutical agent measured in the pulmonary artery. In some cases, the AUC_{Last} can be calculated from plasma concentration of the antiarrhythmic pharmaceutical agent measured in the vein (e.g., femoral vein). In some cases, the AUC_{Last} can be measured in a human PK/PD study.

In some cases, the distribution t_{1/2} of the antiarrhythmic pharmaceutical agent administered via inhalation can be from about 0.1 minute to about 15 minutes, such as from about 0.1-0.5, 0.1-1, 0.1-1.5, 0.1-2, 0.1-2.5, 0.1-2.6, 0.1-2.7, 0.1-2.8, 0.1-2.9, 0.1-3, 0.1-3.1, 0.1-3.2, 0.1-3.3, 0.1-3.4, 0.1-3.5, 0.1-3.6, 0.1-3.7, 0.1-3.8, 0.1-3.9, 0.1-4, 0.1-4.1, 0.1-4.2, 0.1-4.3, 0.1-4.4, 0.1-4.5, 0.1-5, 0.1-5.5, 0.1-6, 0.1-7, 0.1-8, 0.1-9, 0.1-10, 0.1-11, 0.1-12, 0.1-13, 0.1-14, 0.1-15, 0.5-1, 0.5-1.5, 0.5-2, 0.5-2.5, 0.5-2.6, 0.5-2.7, 0.5-2.8, 0.5-2.9, 0.5-3, 0.5-3.1, 0.5-3.2, 0.5-3.3, 0.5-3.4, 0.5-3.5, 0.5-3.6, 0.5-3.7, 0.5-3.8, 0.5-3.9, 0.5-4, 0.5-4.1, 0.5-4.2, 0.5-4.3, 0.5-4.4, 0.5-4.5, 0.5-5, 0.5-5.5, 0.5-6, 0.5-7, 0.5-8, 0.5-9, 0.5-10, 0.5-11, 0.5-12, 0.5-13, 0.5-14, 0.5-15, 1-1.5, 1-2, 1-2.5, 1-2.6, 1-2.7, 1-2.8, 1-2.9, 1-3, 1-3.1, 1-3.2, 1-3.3, 1-3.4, 1-3.5, 1-3.6, 1-3.7, 1-3.8, 1-3.9, 1-4, 1-4.1, 1-4.2, 1-4.3, 1-4.4, 1-4.5, 1-5, 1-5.5, 1-6, 1-7, 1-8, 1-9, 1-10, 1-11, 1-12, 1-13, 1-14, 1-15, 1.5-2, 1.5-2.5, 1.5-2.6, 1.5-2.7, 1.5-2.8, 1.5-2.9, 1.5-3, 1.5-3.1, 1.5-3.2, 1.5-3.3, 1.5-3.4, 1.5-3.5, 1.5-3.6, 1.5-3.7, 1.5-3.8, 1.5-3.9, 1.5-4, 1.5-4.1, 1.5-4.2, 1.5-4.3, 1.5-4.4, 1.5-4.5, 1.5-5, 1.5-5.5, 1.5-6, 1.5-7, 1.5-8, 1.5-9, 1.5-10, 1.5-11, 1.5-12, 1.5-13, 1.5-14, 1.5-15, 2-2.5, 2-2.6, 2-2.7, 2-2.8, 2-2.9, 2-3, 2-3.1, 2-3.2, 2-3.3, 2-3.4, 2-3.5, 2-3.6, 2-3.7, 2-3.8, 2-3.9, 2-4, 2-4.1, 2-4.2, 2-4.3, 2-4.4, 2-4.5, 2-5, 2-5.5, 2-6, 2-7, 2-8, 2-9, 2-10, 2-11, 2-12, 2-13, 2-14, 2-15, 2.5-2.6, 2.5-2.7, 2.5-2.8, 2.5-2.9, 2.5-3, 2.5-3.1, 2.5-3.2, 2.5-3.3, 2.5-3.4, 2.5-3.5, 2.5-3.6, 2.5-3.7, 2.5-3.8, 2.5-3.9, 2.5-4, 2.5-4.1, 2.5-4.2, 2.5-4.3, 2.5-4.4, 2.5-4.5, 2.5-5, 2.5-5.5, 2.5-6, 2.5-7, 2.5-8, 2.5-9, 2.5-10, 2.5-11, 2.5-12, 2.5-13, 2.5-14, 2.5-15, 2.6-2.7, 2.6-2.8, 2.6-2.9, 2.6-3, 2.6-3.1, 2.6-3.2, 2.6-3.3, 2.6-3.4, 2.6-3.5, 2.6-3.6, 2.6-3.7, 2.6-3.8, 2.6-3.9, 2.6-4, 2.6-4.1, 2.6-4.2, 2.6-4.3, 2.6-4.4, 2.6-4.5, 2.6-5, 2.6-5.5, 2.6-6, 2.6-7, 2.6-8, 2.6-9, 2.6-10, 2.6-11, 2.6-12, 2.6-13, 2.6-14, 2.6-15, 2.7-2.8, 2.7-2.9, 2.7-3, 2.7-3.1, 2.7-3.2, 2.7-3.3, 2.7-3.4, 2.7-3.5, 2.7-3.6, 2.7-3.7, 2.7-3.8, 2.7-3.9, 2.7-4, 2.7-4.1, 2.7-4.2, 2.7-4.3, 2.7-4.4, 2.7-4.5, 2.7-5, 2.7-5.5, 2.7-6, 2.7-7, 2.7-8, 2.7-9, 2.7-10, 2.7-11, 2.7-12, 2.7-13, 2.7-14, 2.7-15, 2.8-2.9, 2.8-3, 2.8-3.1, 2.8-3.2, 2.8-3.3, 2.8-3.4, 2.8-3.5, 2.8-3.6, 2.8-3.7, 2.8-3.8, 2.8-3.9, 2.8-4, 2.8-4.1, 2.8-4.2, 2.8-4.3, 2.8-4.4, 2.8-4.5, 2.8-5, 2.8-5.5, 2.8-6, 2.8-7, 2.8-8, 2.8-9, 2.8-10, 2.8-11, 2.8-12, 2.8-13, 2.8-14, 2.8-15, 2.9-3, 2.9-3.1, 2.9-3.2, 2.9-3.3, 2.9-3.4, 2.9-3.5, 2.9-3.6, 2.9-3.7, 2.9-3.8, 2.9-3.9, 2.9-4, 2.9-4.1, 2.9-4.2, 2.9-4.3, 2.9-4.4, 2.9-4.5, 2.9-5, 2.9-5.5, 2.9-6, 2.9-7, 2.9-8, 2.9-9, 2.9-10, 2.9-11, 2.9-12, 2.9-13, 2.9-14, 2.9-15, 3-3.1, 3-3.2, 3-3.3, 3-3.4, 3-3.5, 3-3.6, 3-3.7, 3-3.8, 3-3.9, 3-4, 3-4.1, 3-4.2, 3-4.3, 3-4.4, 3-4.5, 3-5, 3-5.5, 3-6, 3-7, 3-8, 3-9, 3-10, or 14-15 min. In some cases, the distribution t_{1/2} of the antiarrhythmic pharmaceutical agent administered via inhalation can be from about 3 to about 5 minutes. In one or more embodiments antiarrhythmic pharmaceutical agent is a class I, class II, class III, or class IV antiarrhythmic. In some embodiments, the antiarrhythmic pharmaceutical agent is a class Ic, antiarrhythmic. In other embodiments, the antiarrhythmic pharmaceutical agent is flecainide or a pharmaceutically acceptable salt thereof.

In some cases, the distribution t_{1/2} can be calculated as the time at which the antiarrhythmic pharmaceutical agent plasma levels decreased to half of what they were at equilibrium due to distribution to tissues throughout the body. In some cases, the distribution t_{1/2} can be calculated as the time it takes for an antiarrhythmic pharmaceutical agent to lose half of its pharmacologic activity. In some cases, the distribution t_{1/2} can be calculated from plasma concentration of the antiarrhythmic pharmaceutical agent measured in the left ventricular chamber. In some cases, the distribution t_{1/2} can be calculated from plasma concentration of the antiarrhythmic pharmaceutical agent measured in the pulmonary artery. In some cases, the distribution t_{1/2} can be calculated from plasma concentration of the antiarrhythmic pharmaceutical agent measured in the vein (e.g., femoral vein). In some cases, the distribution t_{1/2} can be measured in a human PK/PD study.

In some cases, the elimination t_{1/2} of the antiarrhythmic pharmaceutical agent administered via inhalation can be from about 1 hour to about 25 hours, such as from about 1-3, 1-5, 1-7, 1-7.5, 1-8, 1-8.5, 1-8.7, 1-8.9, 1-9.1, 1-9.3, 1-9.5, 1-9.7, 1-9.9, 1-10.1, 1-10.3, 1-10.5, 1-10.7, 1-10.9, 1-11.1, 1-11.3, 1-11.5, 1-11.7, 1-11.9, 1-12.1, 1-12.5, 1-13, 1-13.5, 1-14, 1-15, 1-16, 1-17, 1-18, 1-19, 1-20, 1-25, 3-5, 3-7, 3-7.5, 3-8, 3-8.5, 3-8.7, 3-8.9, 3-9.1, 3-9.3, 3-9.5, 3-9.7, 3-9.9, 3-10.1, 3-10.3, 3-10.5, 3-10.7, 3-10.9, 3-11.1, 3-11.3, 3-11.5, 3-11.7, 3-11.9, 3-12.1, 3-12.5, 3-13, 3-13.5, 3-14, 3-15, 3-16, 3-17, 3-18, 3-19, 3-20, 3-25, 5-7, 5-7.5, 5-8, 5-8.5, 5-8.7, 5-8.9, 5-9.1, 5-9.3, 5-9.5, 5-9.7, 5-9.9, 5-10.1, 5-10.3, 5-10.5, 5-10.7, 5-10.9, 5-11.1, 5-11.3, 5-11.5, 5-11.7, 5-11.9, 5-12.1, 5-12.5, 5-13, 5-13.5, 5-14, 5-15, 5-16, 5-17, 5-18, 5-19, 5-20, 5-25, 7-7.5, 7-8, 7-8.5, 7-8.7, 7-8.9, 7-9.1, 7-9.3, 7-9.5, 7-9.7, 7-9.9, 7-10.1, 7-10.3, 7-10.5, 7-10.7, 7-10.9, 7-11.1, 7-11.3, 7-11.5, 7-11.7, 7-11.9, 7-12.1, 7-12.5, 7-13, 7-13.5, 7-14, 7-15, 7-16, 7-17, 7-18, 7-19, 7-20, 7-25, 7.5-8, 7.5-8.5, 7.5-8.7, 7.5-8.9, 7.5-9.1, 7.5-9.3, 7.5-9.5, 7.5-9.7, 7.5-9.9, 7.5-10.1, 7.5-10.3, 7.5-10.5, 9.3-10.5, 9.3-10.7, 9.3-10.9, 9.3-11.1, 9.3-11.3, 9.3-11.5, 9.3-11.7, 9.3-11.9, 9.3-12.1, 9.3-12.5, 9.3-13, 9.3-13.5, 9.3-14, 9.3-15, 9.3-16, 9.3-17, 9.3-18, 9.3-19, 9.3-20, 9.3-25, 9.5-9.7, 9.5-9.9, 9.5-10.1, 9.5-10.3, 9.5-10.5, 9.5-10.7, 9.5-10.9, 9.5-11.1, 9.5-11.3, 9.5-11.5, 9.5-11.7, 9.5-11.9, 9.5-12.1, 9.5-12.5, 9.5-13, 9.5-13.5, 9.5-14, 9.5-15, 9.5-16, 9.5-17, 9.5-18, 9.5-19, 9.5-20, 9.5-25, 9.7-9.9, 9.7-10.1, 9.7-10.3, 9.7-10.5, 9.7-10.7, 9.7-10.9, 9.7-11.1, 9.7-11.3, 9.7-11.5, 9.7-11.7, 9.7-11.9, 9.7-12.1, 9.7-12.5, 9.7-13, 9.7-13.5, 9.7-14, 9.7-15, 9.7-16, 9.7-17, 9.7-18, 9.7-19, 9.7-20, 9.7-25, 9.9-10.1, 9.9-10.3, 9.9-10.5, 9.9-10.7, 9.9-10.9, 9.9-11.1, 9.9-11.3, 9.9-11.5, 9.9-11.7, 9.9-11.9, 9.9-12.1, 9.9-12.5, 9.9-13, 9.9-13.5, 9.9-14, 9.9-15, 9.9-16, 9.9-17, 9.9-18, 9.9-19, 9.9-20, 9.9-25, 10.1-10.3, 10.1-10.5, 10.1-10.7, 10.1-10.9, 10.1-11.1, 10.1-11.3, 10.1-11.5, 10.1-11.7, 10.1-11.9, 10.1-12.1, 10.1-12.5, 10.1-13, 10.1-13.5, 10.1-14, 10.1-15, 10.1-16, 10.1-17, 10.1-18, 10.1-19, 10.1-20, 10.1-25, 10.3-10.5, 10.3-10.7, 10.3-10.9, 10.3-11.1, 10.3-11.3, 10.3-11.5, 10.3-11.7, 10.3-11.9, 10.3-12.1, 10.3-12.5, 10.3-13, 10.3-13.5, 10.3-14, 10.3-15, 10.3-16, 10.3-17, 10.3-18, 10.3-19, 10.3-20, 12.1-17, 12.1-18, 12.1-19, 12.1-20, 12.1-25, 12.5-13, 12.5-13.5, 12.5-14, 12.5-15, 12.5-16, 12.5-17, 12.5-18, 12.5-19, 12.5-20, 12.5-25, 13-13.5, 13-14, 13-15, 13-16, 13-17, 13-18, 13-19, 13-20, 13-25, 13.5-14, 13.5-15, 13.5-16, 13.5-17, 13.5-18, 13.5-19, 13.5-20, 13.5-25, 14-15, 14-16, 14-17, 14-18, 14-19, 14-20, 14-25, 15-16, 15-17, 15-18, 15-19, 15-20, 15-25, 16-17, 16-18, 16-19, 16-20, 16-25, 17-18, 17-19, 17-20, 17-25, 18-19, 18-20, 18-25, 19-20, 19-25, or 20-25 hours. In some cases, the elimination t_{1/2} of the antiarrhythmic pharmaceutical agent administered via inhalation can be from about 8.5 to about 10.5 hours. In one or more embodiments antiarrhythmic pharmaceutical agent is a class I, class II, class III, or class IV antiarrhythmic. In some embodiments, the antiarrhythmic pharmaceutical agent is a class Ic, antiarrhythmic. In other embodiments, the antiarrhythmic pharmaceutical agent is flecainide or a pharmaceutically acceptable salt thereof.

In some cases, the elimination t_{1/2} can be calculated as the time at which the antiarrhythmic pharmaceutical agent plasma levels decreased to half of what they were at equilibrium due to metabolism and elimination. In some cases, the elimination t_{1/2} can be calculated from plasma concentration of the antiarrhythmic pharmaceutical agent measured in the left ventricular chamber. In some cases, the elimination t_{1/2} can be calculated from plasma concentration of the antiarrhythmic pharmaceutical agent measured in the pulmonary artery. In some cases, the elimination t_{1/2} can be calculated from plasma concentration of the antiarrhythmic pharmaceutical agent measured in the vein (e.g., femoral vein). In some cases, the elimination t_{1/2} can be measured in a human PK/PD study.

In some cases, the maximum change in QRS interval duration (AQRS) following the antiarrhythmic pharmaceutical agent administered via inhalation can be from about 0.01 msec to about 100 msec, such as from about 0.01-0.1, 0.01-0.5, 0.01-1, 0.01-1.5, 0.01-2, 0.01-2.5, 0.01-3, 0.01-3.5, 0.01-4, 0.01-4.5, 0.01-5, 0.01-5.5, 0.01-6, 0.01-8, 0.01-10, 0.01-15, 0.01-20, 0.01-25, 0.01-30, 0.01-40, 0.01-50, 0.01-60, 0.01-70, 0.01-80, 0.01-90, 0.01-100, 0.1-0.5, 0.1-1, 0.1-1.5, 0.1-2, 0.1-2.5, 0.1-3, 0.1-3.5, 0.1-4, 0.1-4.5, 0.1-5, 0.1-5.5, 0.1-6, 0.1-8, 0.1-10, 0.1-15, 0.1-20, 0.1-25, 0.1-30, 0.1-40, 0.1-50, 0.1-60, 0.1-70, 0.1-80, 0.1-90, 0.1-100, 0.5-1, 0.5-1.5, 0.5-2, 0.5-2.5, 0.5-3, 0.5-3.5, 0.5-4, 0.5-4.5, 0.5-5, 0.5-5.5, 0.5-6, 0.5-8, 0.5-10, 0.5-15, 0.5-20, 0.5-25, 0.5-30, 0.5-40, 0.5-50, 0.5-60, 0.5-70, 0.5-80, 0.5-90, 0.5-100, 1-1.5, 1-2, 1-2.5, 1-3, 1-3.5, 1-4, 1-4.5, 1-5, 1-5.5, 1-6, 1-8, 1-10, 1-15, 1-20, 1-25, 1-30, 1-40, 1-50, 1-60, 1-70, 1-80, 1-90, 1-100, 1.5-2, 1.5-2.5, 1.5-3, 1.5-3.5, 1.5-4, 1.5-4.5, 1.5-5, 1.5-5.5, 1.5-6, 1.5-8, 1.5-10, 1.5-15, 1.5-20, 1.5-25, 1.5-30, 1.5-40, 1.5-50, 1.5-60, 1.5-70, 1.5-80, 1.5-90, 1.5-100, 2-2.5, 2-3, 2-3.5, 2-4, 2-4.5, 2-5, 2-5.5, 2-6, 2-8, 2-10, 2-15, 2-20, 2-25, 2-30, 2-40, 2-50, 2-60, 2-70, 2-80, 2-90, 2-100, 2.5-3, 2.5-3.5, 2.5-4, 2.5-4.5, 2.5-5, 2.5-5.5, 2.5-6, 2.5-8, 2.5-10, 2.5-15, 2.5-20, 2.5-25, 2.5-30, 2.5-40, 2.5-50, 2.5-60, 2.5-70, 2.5-80, 2.5-90, 2.5-100, 3-3.5, 3-4, 3-4.5, 3-5, 3-5.5, 3-6, 3-8, 3-10, 3-15, 3-20, 3-25, 3-30, 3-40, 3-50, 3-60, 3-70, 3-80, 3-90, 3-100, 3.5-4, 3.5-4.5, 3.5-5, 3.5-5.5, 3.5-6, 3.5-8, 3.5-10, 3.5-15, 3.5-3.20, 3.5-3.25, 3.5-3.30, 3.5-40, 3.5-50, 3.5-60, 3.5-70, 3.5-80, 3.5-90, 3.5-100, 4-4.5, 4-5, 4-5.5, 4-6, 4-8, 4-10, 4-15, 4-20, 4-25, 4-30, 4-40, 4-50, 4-60, 4-70, 4-80, 4-90, 4-100, 4.5-5, 4.5-5.5, 4.5-6, 4.5-8, 4.5-10, 4.5-15, 4.5-20, 4.5-25, 4.5-30, 4.5-4.50, 4.5-50, 4.5-60, 4.5-70, 4.5-80, 4.5-90, 4.5-100, 5-5.5, 5-6, 5-8, 5-10, 5-15, 5-20, 5-25, 5-30, 5-40, 5-50, 5-60, 5-70, 5-80, 5-90, 5-100, 5.5-6, 5.5-8, 5.5-10, 5.5-15, 5.5-20, 5.5-25, 5.5-30, 5.5-40, 5.5-50, 5.5-60, 5.5-70, 5.5-80, 5.5-90, 5.5-100, 6-8, 6-10, 6-15, 6-20, 6-25, 6-30, 6-40, 6-50, 6-60, 6-70, 6-80, 6-90, 6-100, 8-10, 8-15, 8-20, 8-25, 8-30, 8-40, 8-50, 8-60, 8-70, 8-80, 8-90, 8-100, 10-15, 10-20, 10-25, 10-30, 10-40, 10-50, 10-60, 10-70, 10-80, 10-90, 10-100, 15-20, 15-25, 15-30, 15-40, 15-50, 15-60, 15-70, 15-80, 15-90, 15-100, 20-25, 20-30, 20-40, 20-50, 20-60, 20-70, 20-80, 20-90, 20-100, 25-30, 25-40, 25-50, 25-60, 25-70, 25-80, 25-90, 25-100, 30-40, 30-50, 30-60, 30-70, 30-80, 30-90, 30-100, 40-50, 40-60, 40-70, 40-80, 40-90, 40-100, 50-60, 50-70, 50-80, 50-90, 50-100, 60-70, 60-80, 60-90, 60-100, 70-80, 70-90, 70-100, 80-90, 80-100, or 90-100 msec. In some cases, the maximum change in QRS interval duration (AQRS) following the antiarrhythmic pharmaceutical agent administered via inhalation can be from about 1 to about 10 msec. In some cases, the maximum change in QRS interval duration (AQRS) following the antiarrhythmic pharmaceutical agent administered via inhalation can be from about 5 to about 20 msec. In some cases, the AQRS can be measured in a human PK/PD study. In the present disclosure, the term “AQRS”, if not referred to with reference to time post-administration of the antiarrhythmic agent, can be used interchangeably with the term “maximum ΔQRS”, e.g. meaning the maximum change in QRS following administration of the antiarrhythmic agent as provided herein. In one or more embodiments antiarrhythmic pharmaceutical agent is a class I, class II, class III, or class IV antiarrhythmic. In some embodiments, the antiarrhythmic pharmaceutical agent is a class Ic, antiarrhythmic. In other embodiments, the antiarrhythmic pharmaceutical agent is flecainide or a pharmaceutically acceptable salt thereof.

In some cases, the time point at which the QRS interval is measured following the antiarrhythmic pharmaceutical agent administration via inhalation to determine the AQRS relative to pre-dose can be from about 0.1 minute to about 450 minutes, such as from about 0.1-1, 0.1-3, 0.1-5, 0.1-10, 0.1-15, 0.1-30, 0.1-45, 0.1-60, 0.1-90, 0.1-120, 0.1-150, 0.1-180, 0.1-210, 0.1-240, 0.1-270, 0.1-300, 0.1-330, 0.1-360, 0.1-390, 0.1-410, 0.1-450, 1-3, 1-5, 1-10, 1-15, 1-30, 1-45, 1-60, 1-90, 1-120, 1-150, 1-180, 1-210, 1-240, 1-270, 1-300, 1-330, 1-360, 1-390, 1-410, 1-450, 3-5, 3-10, 3-15, 3-30, 3-45, 3-60, 3-90, 3-120, 3-150, 3-180, 3-210, 3-240, 3-270, 3-300, 3-330, 3-360, 3-390, 3-410, 3-450, 5-10, 5-15, 5-30, 5-45, 5-60, 5-90, 5-120, 5-150, 5-180, 5-210, 5-240, 5-270, 5-300, 5-330, 5-360, 5-390, 5-410, 5-450, 10-15, 10-30, 10-45, 10-60, 10-90, 10-120, 10-150, 10-180, 10-210, 10-240, 10-270, 10-300, 10-330, 10-360, 10-390, 10-410, 10-450, 15-30, 15-45, 15-60, 15-90, 15-120, 15-150, 15-180, 15-210, 15-240, 15-270, 15-300, 15-330, 15-360, 15-390, 15-410, 15-450, 30-45, 30-60, 30-90, 30-120, 30-150, 30-180, 30-210, 30-240, 30-270, 30-300, 30-330, 30-360, 30-390, 30-410, 30-450, 45-60, 45-90, 45-120, 45-150, 45-180, 45-210, 45-240, 45-270, 45-300, 45-330, 45-360, 45-390, 45-410, 45-450, 60-90, 60-120, 60-150, 60-180, 60-210, 60-240, 60-270, 60-300, 60-330, 60-360, 60-390, 60-410, 60-450, 90-120, 90-150, 90-180, 90-210, 90-240, 90-270, 90-300, 90-330, 90-360, 90-390, 90-410, 90-450, 120-150, 120-180, 120-210, 120-240, 120-270, 120-300, 120-330, 120-360, 120-390, 120-410, 120-450, 150-180, 150-210, 150-240, 150-270, 150-300, 150-330, 150-360, 150-390, 150-410, 150-450, 180-210, 180-240, 180-270, 180-300, 180-330, 180-360, 180-390, 180-410, 180-450, 210-240, 210-270, 210-300, 210-330, 210-360, 210-390, 210-410, 210-450, 240-270, 240-300, 240-330, 240-360, 240-390, 240-410, 240-450, 270-300, 270-330, 270-360, 270-390, 270-410, 270-450, 300-330, 300-360, 300-390, 300-410, 300-450, 330-360, 330-390, 330-410, 330-450, 360-390, 360-410, 360-450, 390-410, 390-450, or 410-450 min.

The antiarrhythmic activity of pharmaceutical agent can be correlated with QRS interval duration. In some examples, the antiarrhythmic pharmaceutical agent administered via inhalation can have higher antiarrhythmic activity as compared to the antiarrhythmic pharmaceutical agent administered by intravenous delivery (e.g., intravenous infusion). In some cases, such a higher antiarrhythmic activity is reflected by a higher ratio of maximum ΔQRS to Cₘₐₓ. For example, given the same Cₘₐₓ, e.g., peak plasma concentration of the antiarrhythmic pharmaceutic agent, inhalation delivery of the antiarrhythmic agent as provided herein can have a higher maximum ΔQRS as compared to intravenous delivery of the same agent. In some cases, the comparison may not be made between corresponding doses via the two different administration routes, for example, inhalation of a first dose of the agent can have a first Cₘₐₓ (Cₘₐₓ₁) and a first maximum ΔQRS (ΔQRSₘₐₓ₁), and intravenous administration of a second dose of the agent can have a second Cₘₐₓ (Cₘₐₓ₂) and a second maximum ΔQRS (ΔQRSₘₐₓ₂). In some cases, Cₘₐₓ₁ and Cₘₐₓ₂ can be similar. In other case, Cₘₐₓ₁ and Cₘₐₓ₂ can be dissimilar. In some examples of the present disclosure, the ratio of ΔQRSₘₐₓ₁ versus Cₘₐₓ₁ can be higher than ΔQRSₘₐₓ₂ versus Cₘₐₓ₂, i.e., ΔQRSₘₐₓ₁/Cₘₐₓ₁>ΔQRSₘₐₓ₂/Cₘₐₓ₂. In some cases, ΔQRSₘₐₓ₁/Cₘₐₓ₁ is at least 1.1 folds, at least 1.2 folds, at least 1.3 folds, at least 1.4 folds, at least 1.5 folds, at least 1.6 folds, at least 1.7 folds, at least 1.8 folds, at least 1.9 folds, at least 2.0 folds, at least 2.1 folds, at least 2.2 folds, at least 2.3 folds, at least 2.4 folds, at least 2.5 folds, at least 2.6 folds, at least 2.7 folds, at least 2.8 folds, at least 2.9 folds, at least 3.0 folds, at least 3.1 folds, at least 3.2 folds, at least 3.3 folds, at least 3.4 folds, at least 3.5 folds, at least 3.6 folds, at least 3.7 folds, at least 3.8 folds, at least 3.9 folds, at least 4.0 folds, at least 4.2 folds, at least 4.4 folds, at least 4.6 folds, at least 4.8 folds, at least 5.0 folds, at least 5.5 folds, at least 6 folds, at least 7 folds, at least 8 folds, at least 9 folds, at least 10 folds, at least 12 folds, at least 15 folds, at least 20 folds, at least 25 folds, or at least 50 folds greater than ΔQRSₘₐₓ₂/Cₘₐₓ₂. In some cases, ΔQRSₘₐₓ₁/Cₘₐₓ₁ is at least 2 folds greater than ΔQRSₘₐₓ₂/Cₘₐₓ₂. In one or more embodiments antiarrhythmic pharmaceutical agent is a class I, class II, class III, or class IV antiarrhythmic. In some embodiments, the antiarrhythmic pharmaceutical agent is a class Ic, antiarrhythmic. In other embodiments, the antiarrhythmic pharmaceutical agent is flecainide or a pharmaceutically acceptable salt thereof.

In some cases, the compositions and methods provided herein confer a reduced negative inotropic burden to the subject receiving inhalational delivery of the antiarrhythmic pharmaceutical agent, e.g., flecainide, as compared to receiving delivery of a corresponding dose of the same agent via a different route (e.g., oral or intravenous delivery). Certain antiarrhythmic drugs can have negative inotropic effect, which can limit their use for acute cardioversion of new-onset paroxysmal atrial fibrillation (AF). For instance, intravenous delivery of flecainide can exert negative inotropic burden to the subject's heart, which can be measured by left ventricular (LV) contractility. In some cases, the negative inotropic burden can be measured by the area under the curve (AUC) of a curve depicting the magnitude of LV contractility (e.g., measured by dP/dt max) and time that it remain below baseline (e.g., baseline level before drug administration or at rest). In some embodiments, the negative inotropic burden of inhalational delivery of a therapeutically effective dose of antiarrhythmic pharmaceutical agent according to the methods described herein is lower than that of a corresponding therapeutically effective dose of the same agent delivered via a different route (e.g., oral or intravenous delivery). For instance, in such embodiments, the negative inotropic burden of inhalational delivery of a therapeutically effective dose of antiarrhythmic pharmaceutical agent according to the methods described herein is at most 90%, 80%, 70%, 60%, 50%, 40%, 30%, 20%, or 10% of that of a corresponding therapeutically effective dose of the same agent delivered via a different route (e.g., oral or intravenous delivery). In some embodiments, the negative inotropic burden of inhalational delivery of a therapeutically effective dose of antiarrhythmic pharmaceutical agent according to the methods described herein is about 90%, 80%, 70%, 60%, 50%, 40%, 30%, 20%, or 10% of that of a corresponding therapeutically effective dose of the same agent delivered via a different route (e.g., oral or intravenous delivery). the negative inotropic burden of inhalational delivery of a therapeutically effective dose of antiarrhythmic pharmaceutical agent according to the methods described herein is about 30% of that of a corresponding therapeutically effective dose of the same agent delivered via a different route (e.g., oral or intravenous delivery). In such embodiments, the corresponding therapeutically effective dose of the antiarrhythmic pharmaceutical agent delivered via the other route can have a similar conversion rate (a percentage of number of effective conversion of arrhythmia to sinus rhythm), e.g., with a variation of less than 20% or 10%, as compared to the therapeutically effective inhalational dose of the same agent.

### Indications and Subjects

Examples of cardiac arrhythmias the methods, compositions, and kits provided herein can treat include, but are not limited to, tachycardia, supraventricular tachycardia (SVT), paroxysmal supraventricular tachycardia (PSVT), atrial fibrillation (AF), paroxysmal atrial fibrillation (PAF), persistent atrial fibrillation, permanent atrial fibrillation, atrial flutter, paroxysmal atrial flutter, and lone atrial fibrillation. In some cases, the methods, compositions, and kits provided herein find use in treating a subject suffering from atrial arrhythmia, e.g., atrial fibrillation.

Thus, the pharmaceutical compositions according to some examples of the present disclosure can be used to treat and/or provide prophylaxis for a broad range of patients. A suitable patient for, receiving treatment and/or prophylaxis as described herein is any mammalian patient in need thereof, preferably such mammal is a human. Examples of subjects include, but are not limited to, pediatric patients, adult patients, and geriatric patients. In some cases, the composition is intended only as a treatment for rapid resolution of symptoms and restoration of normal sinus rhythm, and is not taken as a preventative, e.g., when the patient is well, there is no need for drug—this can increase the benefit-risk ratio of the therapy and overall safety due to the sporadic or intermittent dosing, and the focus on reducing disabling symptoms and restoring sinus rhythm only when needed.

The dosage necessary and the frequency of dosing of the antiarrhythmic pharmaceutical agent depend on the composition and concentration of the antiarrhythmic pharmaceutical agent within the composition. In some cases, the dose is less than about 10%, 20%, 30%, 40%, 50%, 60%, 70%, 80%, 90%, or 95% of its normal intravenous dose. In some cases, the dose is about 5% to about 10%, is about 10% to about 20%, is about 20% to about 30%, is about 30% to about 40%, is about 50% to about 60%, is about 60% to about 70%, is about 70% to about 80%, is about 80% to about 90%, or is about 90% to about 95% of the intravenous dose.

Pharmaceutical compositions disclosed herein can be more effective in subjects that include or lack certain physiological or demographic factors, such as, for example, age at clinical presentation, certain hemodynamic criteria, electrophysiological features, and prior treatments. In some embodiments, a subject treated with a pharmaceutical composition of the disclosure suffers from an atrial fibrillation with an onset that occurred within 48 hours prior to the treating. In some embodiments, a subject treated with a pharmaceutical composition of the disclosure suffers from an atrial fibrillation with an onset that occurred from 1 hour to 48 hours prior to the treating. In some embodiments, a subject treated with a pharmaceutical composition of the disclosure suffers from recurrent atrial fibrillation. In some embodiments, a subject treated with a pharmaceutical composition of the disclosure has undergone cardiac ablation no less than 3 months prior to the treating. In some embodiments, a subject treated with a pharmaceutical composition of the disclosure has an ongoing prescription for an oral antiarrhythmic medication for atrial fibrillation. In some embodiments, the oral antiarrhythmic medication is flecainide, or a pharmaceutically acceptable salt thereof.

In some embodiments, a subject treated with a pharmaceutical composition of the disclosure is over 17 years in age. In some embodiments, a subject treated with a pharmaceutical composition of the disclosure is no more than 85 years in age. In some embodiments, a subject treated with a pharmaceutical composition of the disclosure is from 18 years old to 85 years old.

In some embodiments, a subject treated with a pharmaceutical composition of the disclosure has a systolic blood pressure that is below 180 mmHg, below 175 mmHg, below 170 mmHg, below 165 mmHg, below 160 mmHg, below 155 mmHg, or below 150 mmHg at the time of the treating. In some cases, when referring to a physiological measurement of the subject, for instance, blood pressure, e.g., systolic blood pressure or diastolic blood pressure, or heart rate, e.g., ventricular rate, the term “at the time of treating” means the measurement is taken from 1 min to 6 hr prior to the treating, for instance, when measured 1 min to 10 min, 1 min to 30 min, 1 min to 60 min, 1 min to 90 min, 1 min to 2 hr, 1 min to 3 hr, 1 min to 4 hr, 1 min to 5 hr, 10 min to 30 min, 10 min to 60 min, 30 min to 60 min, 30 min to 90 min, 30 min to 2 hr, 1 hr to 2 hr, or 2 hr to 3 hr prior to the treating. In some cases, the physiological measurement, for instance, the measurement of the systolic blood pressure or the ventricular rate of the subject provides a basis for an informed decision as to whether or not the subject is to be treated with the subject pharmaceutical composition and method. In some embodiments, a subject treated with a pharmaceutical composition of the disclosure has a systolic blood pressure that is greater than 70 mmHg, greater than 75 mmHg, greater than 80 mmHg, greater than 85 mmHg, greater than 90 mmHg, greater than 95 mmHg, greater than 100 mmHg, greater than 105 mmHg, greater than 110 mmHg, greater than 115 mmHg, or greater than 120 mmHg at the time of the treating.

In some embodiments, a subject treated with a pharmaceutical composition of the disclosure has a systolic blood pressure that is from about 60 mmHg to about 180 mmHg, from about 65 mmHg to about 180 mmHg, from about 70 mmHg to about 180 mmHg, from about 75 mmHg to about 180 mmHg, from about 80 mmHg to about 180 mmHg, from about 85 mmHg to about 180 mmHg, from about 90 mmHg to about 180 mmHg, from about 95 mmHg to about 180 mmHg, from about 100 mmHg to about 180 mmHg, from about 105 mmHg to about 180 mmHg, from about 110 mmHg to about 180 mmHg, from about 115 mmHg to about 180 mmHg, from about 120 mmHg to about 180 mmHg, from about 70 mmHg to about 175 mmHg, from about 70 mmHg to about 170 mmHg, from about 70 mmHg to about 165 mmHg, from about 70 mmHg to about 160 mmHg, from about 70 mmHg to about 155 mmHg, from about 70 mmHg to about 150 mmHg, from about 80 mmHg to about 165 mmHg, from about 90 mmHg to about 165 mmHg, from about 100 mmHg to about 165 mmHg, from about 70 mmHg to about 160 mmHg, from about 70 mmHg to about 160 mmHg, from about 75 mmHg to about 160 mmHg, from about 80 mmHg to about 160 mmHg, from about 85 mmHg to about 160 mmHg, from about 90 mmHg to about 160 mmHg, from about 95 mmHg to about 160 mmHg, from about 100 mmHg to about 160 mmHg, from about 70 mmHg to about 155 mmHg, from about 75 mmHg to about 155 mmHg, or from about 80 mmHg to about 155 mmHg at the time of treating.

In some embodiments, a subject treated with a pharmaceutical composition of the disclosure has a ventricular rate that is at least about 50 BPM, at least about 55 BPM, at least about 60 BPM, at least about 65 BPM, at least about 70 BPM, at least about 75 BPM, at least about 80 BPM, at least about 85 BPM, at least about 90 BPM, at least about 95 BPM, or at least about 100 BPM at the time of treating. In some embodiments, a subject treated with a pharmaceutical composition of the disclosure has a ventricular rate that is no greater than about 200 BPM, no greater than about 190 BPM, no greater than about 180 BPM, no greater than about 175 BPM, no greater than about 170 BPM, no greater than about 165 BPM, no greater than about 160 BPM, no greater than about 155 BPM, no greater than about 150 BPM, no greater than about 145 BPM, or no greater than about 140 BPM at the time of treating.

In some embodiments, a subject treated with a pharmaceutical composition of the disclosure has a ventricular rate that is from about 50 BPM to about 200 BPM, 50 BPM to about 180 BPM, from about 55 BPM to about 180 BPM, from about 60 BPM to about 180 BPM, from about 65 BPM to about 180 BPM, from about 70 BPM to about 180 BPM, from about 75 BPM to about 180 BPM, from about 80 BPM to about 180 BPM, about 85 BPM to about 180 BPM, about 95 BPM to about 180 BPM, about 100 BPM to about 180 BPM, from about 50 BPM to about 175 BPM, from about 50 BPM to about 170 BPM, from about 50 BPM to about 165 BPM, from about 50 BPM to about 160 BPM, from about 50 BPM to about 155 BPM, from about 70 BPM to about 175 BPM, about 70 BPM to about 170 BPM, about 70 BPM to about 165 BPM, about 70 BPM to about 160 BPM, about 70 BPM to about 155 BPM, about 75 BPM to about 180 BPM, about 75 BPM to about 175 BPM, about 75 BPM to about 170 BPM, about 75 BPM to about 165 BPM, about 75 BPM to about 160 BPM, about 75 BPM to about 155 BPM, about 80 BPM to about 175 BPM, about 80 BPM to about 170 BPM, about 80 BPM to about 165 BPM, about 80 BPM to about 160 BPM, about 80 BPM to about 155 BPM, about 80 BPM to about 150 BPM, about 80 BPM to about 145 BPM, about 85 BPM to about 155 BPM, about 90 BPM to about 155 BPM, about 95 BPM to about 155 BPM, or about 100 BPM to about 155 BPM at the time of treating.

In some embodiments, a subject treated with a pharmaceutical composition of the disclosure has not been treated with antiarrhythmic drugs or electrical cardioversion since onset of an episode of atrial arrhythmia for which the pharmaceutical composition is being administered. In some embodiments, a subject treated with a pharmaceutical composition of the disclosure does not exhibit acute decompensated heart failure at the time of treating. In some embodiments, a subject treated with a pharmaceutical composition of the disclosure does not have heart failure with reduced ejection fraction or a history thereof. In some embodiments, a subject treated with a pharmaceutical composition of the disclosure does not have myocardial ischemia or a history thereof. In some embodiments, a subject treated with a pharmaceutical composition of the disclosure does not have myocardial infarction or a history thereof. In some embodiments, a subject treated with a pharmaceutical composition of the disclosure has not exhibited myocardial infarction (MI) within 3 months prior to administration of the pharmaceutical composition. In some embodiments, a subject treated with a pharmaceutical composition of the disclosure does not exhibit uncorrected severe aortic or mitral stenosis at the time of treating. In some embodiments, a subject treated with a pharmaceutical composition of the disclosure does not exhibit hypertrophic cardiomyopathy with outflow tract obstruction at the time of treating. In some embodiments, a subject treated with a pharmaceutical composition of the disclosure does not have persistent atrial fibrillation or a history thereof. In some embodiments, a subject treated with a pharmaceutical composition of the disclosure does not exhibit atrial flutter at the time of treating. In some embodiments, a subject treated with a pharmaceutical composition of the disclosure has not exhibited an episode of atrial flutter within 6 months prior to the treating. In some embodiments, a subject treated with a pharmaceutical composition of the disclosure does not exhibit abnormal left ventricular ejection fraction at the time of treating. In some embodiments, a subject treated with a pharmaceutical composition of the disclosure does not exhibit heart failure that is class 2 or greater as according to New York Heart Association Functional Classification at the time of treating.

In some embodiments, a subject treated with a pharmaceutical composition of the disclosure is hemodynamically stable, has a systolic blood pressure that is greater than about 90 mmHg, has ventricular rate from about 70 BPM to about 170 BPM at the time of treating, and does not have a condition or a history of a condition that is: myocardial infarction, myocardial ischemia, atrial stenosis, hypertrophic cardiomyopathy, and heart failure with reduced ejection fraction.

In some embodiments, a subject treated with a pharmaceutical composition of the disclosure does not have Long QT syndrome, Conduction disease (e.g. second- or third-degree heart block, bundle branch block), Sick sinus syndrome, Brugada Syndrome, Torsades de pointes (TdP), or a histories thereof. In some embodiments, a subject treated with a pharmaceutical composition of the disclosure does not exhibit at the time of treating an ECG-related feature that is: a QTc interval greater than 480 msec (estimated by the Fridericia's formula); a QRS duration greater than 105 ms; monomorphic or polymorphic ventricular tachycardias that are either sustained or not sustained; and excessive premature ventricular contractions greater than 20 multi-focal PVC's per hour (ventricular extrasystoles); or a predominantly paced heart rhythm.

In some embodiments, a subject treated with a pharmaceutical composition of the disclosure does not exhibit severe renal impairment, wherein a eGFR of the subject is less than 30 mL/min/1.73 m² at the time of treating. In some embodiments, a subject treated with a pharmaceutical composition of the disclosure is not on dialysis at the time of treating. In some embodiments, a subject treated with a pharmaceutical composition of the disclosure does not exhibit abnormal liver function at the time of treating. In some embodiments, the abnormal liver function is hepatic disease or biochemical evidence of significant liver derangement. In some embodiments, a subject treated with a pharmaceutical composition of the disclosure does not exhibit uncorrected hypokalemia at the time of treating. In some embodiments, a subject treated with a pharmaceutical composition of the disclosure does not exhibit a serum potassium less than 3.6 mEq/L at the time of treating.

In some embodiments, a subject treated with a pharmaceutical composition of the disclosure does not exhibit an established pulmonary disease in need of inhalation medication at the time of treating. In some embodiments, a subject treated with a pharmaceutical composition of the disclosure does not have a hypersensitivity to flecainide acetate or any of its active metabolites, or a history thereof. In some embodiments, a subject treated with a pharmaceutical composition of the disclosure is not concomitantly administered a systemic drug that is an inhibitor of CYP 2D6. In some embodiments, the inhibitor of CYP 2D6 is an antidepressant, a neuroleptic, or an antihistamine. In some embodiments, the inhibitor of CYP 2D6 is propranolol or ritonavir. In some embodiments, a subject treated with a pharmaceutical composition of the disclosure is not concomitantly administered a systemic drug that is a CYP 2D6 inducer. In some embodiments, the CYP 2D6 inducer is phenytoin, phenobarbital, or carbamazepine.

In some embodiments, a subject treated with a pharmaceutical composition of the disclosure has not been treated with a Class I or a Class II antiarrhythmic drug within a week prior to administration of the pharmaceutical composition. In some embodiments, a subject treated with a pharmaceutical composition of the disclosure with an ongoing episode of atrial fibrillation has not been treated with a Class I or a Class III antiarrhythmic drug since onset of the ongoing episode. In some embodiments, a subject treated with a pharmaceutical composition of the disclosure is administered no more than 320 mg flecainide or a pharmaceutically acceptable salt thereof per day from any source. In some embodiments, a subject treated with a pharmaceutical composition of the disclosure with an ongoing episode of atrial fibrillation has not been treated with electrical cardioversion since onset of the ongoing episode. In some embodiments, a subject treated with a pharmaceutical composition of the disclosure has not been treated with amiodarone within 12 weeks prior to administration of the pharmaceutical composition. In some embodiments, a subject treated with a pharmaceutical composition of the disclosure has not been considered high risk for stroke based on screening coagulation panel. In some embodiments, a subject treated with a pharmaceutical composition of the disclosure does not exhibit a CHA2DS2-VASc score greater than 2.

In some embodiments, a subject treated with a pharmaceutical composition of the disclosure does not exhibit a congenital heart disease at the time of treating. In some embodiments, a subject treated with a pharmaceutical composition of the disclosure does not exhibit a history of refractory atrial fibrillation that has been pharmacologically or electrically cardioverted. In some embodiments, a subject treated with a pharmaceutical composition of the disclosure does not exhibit atrial fibrillation that is secondary to electrolyte imbalance, thyroid disease, or a non-cardiovascular cause at the time of treating. In some embodiments, a subject treated with a pharmaceutical composition of the disclosure does not exhibit syncope at the time of treating.

In some embodiments, a subject treated with a pharmaceutical composition of the disclosure does not exhibit any serious or life threatening medical condition other than cardiac arrhythmia at the time of treating. In some embodiments, a subject treated with a pharmaceutical composition of the disclosure does not exhibit an acute pathogenic infection at the time of treating.

In some embodiments, a subject treated with a pharmaceutical composition of the disclosure has not exhibited a drug or alcohol dependence within 12 months prior to administration of the pharmaceutical composition. In some embodiments, a subject treated with a pharmaceutical composition of the disclosure does not exhibit a body mass index greater than 40 Kg/m² at the time of treating.

In some embodiments, a subject treated with a pharmaceutical composition of the disclosure:
(i) suffers from an atrial fibrillation with an onset that occurred from 1 hour to 48 hours prior to the treating;
(ii) has undergone cardiac ablation no less than 3 months prior to the treating, has an ongoing prescription for oral flecainide or a pharmaceutically acceptable salt thereof, or suffers from recurrent atrial fibrillation;
(iii) is from 18 years old to 85 years old;
(iv) has a systolic blood pressure that is from about 100 mmHg to about 160 mmHg at the time of treating; and
(v) has a ventricular rate that is from about 80 BPM to about 155 BPM at the time of treating,
   wherein the subject does not exhibit:
(a) abnormal left ventricular ejection fraction within 6 months prior to the treating;
(b) heart failure that is class 2 or greater as classified by New York Heart Association Functional Classification within 6 months prior to the treating;
(c) myocardial infarction or a history thereof;
(d) hemodynamic or cardiac instability at the time of treating; and
(e) an episode of atrial flutter within 6 months prior to the treating, wherein the subject has not undergone cardiac surgery for any of the conditions of (a)-(e) within 6 months prior to the treating.

In some embodiments, the drug mass nebulization rate contributes to the efficiency of the inhalation therapy, as demonstrated in studies in human subjects for the pharmacokinetics and pharmacodynamics of inhalation administration of flecainide acetate (e.g., clinical studies FLE-001, FLE-003 (in healthy subjects) and FLE-002 (patients) sponsored by InCarda Therapeutics, Inc.). In some embodiments, human subjects cannot be expected to inhale the nebulized drug solution continuously for longer than approximately 4.5 minutes without a break. Long inhalation duration (e.g., longer than 5 minutes) can result in fatigue, inadequate or poor compliance with proper inhalation maneuver in some subjects, which can lead to insufficient delivery of drug to the lung, and stress. In some cases, stress in some subjects, e.g., stress induced by long inhalation duration or other discomfort, can lead to a rise in sympathetic tone, which can render cardioversion more difficult.

In some cases, a fast drug mass nebulization rate minimizes inhalation time for an effective dose. The drug mass nebulization rate can be strongly influenced by the drug concentration in the nebulization solution. In a nebulized product, the drug delivery rate can be constrained by the ability of the device to produce a nebulized cloud with an appropriate droplet size for inhalation (e.g., less than 5 microns). On the other hand, when the aerosolization rate is too high and the cloud too dense, the small nebulized droplets can coalesce into larger droplets which tend to deposit in the mouth and throat and do not reach the lungs.

### Pharmaceutical Compositions, Formulations, and Kits

In one aspect, provided herein are pharmaceutical composition for treatment of a heart condition, e.g., cardiac arrhythmia, e.g., atrial arrhythmia.

In another aspect, the present disclosure provides a pharmaceutical composition comprising a therapeutically effective amount of an antiarrhythmic agent for use in treating a patient suffering from a heart condition, wherein said patient has a body mass index (BMI) of at most 40 kg/m², wherein said pharmaceutical composition is administered to said subject via inhalation.

In another aspect, the present disclosure provides an inhalable pharmaceutical composition comprising a therapeutically effective amount of an antiarrhythmic agent for use in treating a patient suffering from a heart condition, wherein said patient has a body mass index (BMI) of at most 40 kg/m².

In another aspect, the present disclosure provides an aerosolized solution comprising a therapeutically effective amount of an antiarrhythmic agent for use in treating a patient suffering from a heart condition, wherein said patient has a body mass index (BMI) of at most 40 kg/m².

The pharmaceutical composition can include a therapeutically effective amount of a salt of flecainide. The therapeutically effective amount of flecainide can be effective for treatment of a heart condition, e.g., cardiac arrhythmia, e.g., atrial arrhythmia, when it is administered to a subject in need thereof via inhalation. In some cases, the therapeutically effective amount of flecainide salt is effective for treatment of atrial arrhythmia by inducing cardioversion when it is administered to a subject in need thereof via inhalation.

In some cases, provided herein are pharmaceutical composition including a therapeutically effective amount of a salt of flecainide. As described herein, in some embodiments, the pharmaceutical composition is in the form of a liquid solution. In some cases, the concentration of flecainide or the salt of flecainide (e.g., flecainide acetate or flecainide hydrochloride) in the pharmaceutical compositions or formulations is about 60 mg/mL to about 200 mg/mL, such as 60 mg/mL to 195 mg/mL, 60 mg/mL to 190 mg/mL, 60 mg/mL to 185 mg/mL, 60 mg/mL to 175 mg/mL, 60 mg/mL to 170 mg/mL, 60 mg/mL to 165 mg/mL, 60 mg/mL to 160 mg/mL, 60 mg/mL to 155 mg/mL, 60 mg/mL to 150 mg/mL, 60 mg/mL to 145 mg/mL, 60 mg/mL to 140 mg/mL, 60 mg/mL to 135 mg/mL, 60 mg/mL to 130 mg/mL, 60 mg/mL to 125 mg/mL, 60 mg/mL to 120 mg/mL, 60 mg/mL to 118 mg/mL, 60 mg/mL to 115 mg/mL, 60 mg/mL to 112 mg/mL, 60 mg/mL to 110 mg/mL, 60 mg/mL to 108 mg/mL, 60 mg/mL to 105 mg/mL, 60 mg/mL to 102 mg/mL, 60 mg/mL to 100 mg/mL, 60 mg/mL to 98 mg/mL, 60 mg/mL to 95 mg/mL, 60 mg/mL to 92 mg/mL, 60 mg/mL to 90 mg/mL, 60 mg/mL to 88 mg/mL, 60 mg/mL to 85 mg/mL, 60 mg/mL to 82 mg/mL60 mg/mL to 80 mg/mL, 60 mg/mL to 78 mg/mL, 60 mg/mL to 75 mg/mL, 60 mg/mL to 72 mg/mL, 60 mg/mL to 70 mg/mL, 70 mg/mL to 195 mg/mL, 70 mg/mL to 190 mg/mL, 70 mg/mL to 185 mg/mL, 70 mg/mL to 175 mg/mL, 70 mg/mL to 170 mg/mL, 70 mg/mL to 165 mg/mL, 70 mg/mL to 160 mg/mL, 70 mg/mL to 155 mg/mL, 70 mg/mL to 150 mg/mL, 70 mg/mL to 145 mg/mL, 70 mg/mL to 140 mg/mL, 70 mg/mL to 135 mg/mL, 70 mg/mL to 130 mg/mL, 70 mg/mL to 125 mg/mL, 70 mg/mL to 120 mg/mL, 70 mg/mL to 118 mg/mL, 70 mg/mL to 115 mg/mL, 70 mg/mL to 112 mg/mL, 70 mg/mL to 110 mg/mL, 70 mg/mL to 108 mg/mL, 70 mg/mL to 105 mg/mL, 70 mg/mL to 102 mg/mL, 70 mg/mL to 100 mg/mL, 70 mg/mL to 98 mg/mL, 70 mg/mL to 95 mg/mL, 70 mg/mL to 92 mg/mL, 70 mg/mL to 90 mg/mL, 70 mg/mL to 88 mg/mL, 70 mg/mL to 85 mg/mL, 70 mg/mL to 82 mg/mL70 mg/mL to 80 mg/mL, 70 mg/mL to 78 mg/mL, 70 mg/mL to 75 mg/mL, 80 mg/mL to 195 mg/mL, 80 mg/mL to 190 mg/mL, 80 mg/mL to 185 mg/mL, 80 mg/mL to 175 mg/mL, 80 mg/mL to 170 mg/mL, 80 mg/mL to 165 mg/mL, 80 mg/mL to 160 mg/mL, 80 mg/mL to 155 mg/mL, 80 mg/mL to 150 mg/mL, 80 mg/mL to 145 mg/mL, 80 mg/mL to 140 mg/mL, 80 mg/mL to 135 mg/mL, 80 mg/mL to 130 mg/mL, 80 mg/mL to 125 mg/mL, 80 mg/mL to 120 mg/mL, 80 mg/mL to 118 mg/mL, 80 mg/mL to 115 mg/mL, 80 mg/mL to 112 mg/mL, 80 mg/mL to 110 mg/mL, 80 mg/mL to 108 mg/mL, 80 mg/mL to 105 mg/mL, 80 mg/mL to 102 mg/mL, 80 mg/mL to 100 mg/mL, 80 mg/mL to 98 mg/mL, 80 mg/mL to 95 mg/mL, 80 mg/mL to 92 mg/mL, 80 mg/mL to 90 mg/mL, 80 mg/mL to 88 mg/mL, 80 mg/mL to 85 mg/mL, 90 mg/mL to 195 mg/mL, 90 mg/mL to 190 mg/mL, 90 mg/mL to 185 mg/mL, 90 mg/mL to 175 mg/mL, 90 mg/mL to 170 mg/mL, 90 mg/mL to 165 mg/mL, 90 mg/mL to 160 mg/mL, 90 mg/mL to 155 mg/mL, 90 mg/mL to 150 mg/mL, 90 mg/mL to 145 mg/mL, 90 mg/mL to 140 mg/mL, 90 mg/mL to 135 mg/mL, 90 mg/mL to 130 mg/mL, 90 mg/mL to 125 mg/mL, 90 mg/mL to 120 mg/mL, 90 mg/mL to 118 mg/mL, 90 mg/mL to 115 mg/mL, 90 mg/mL to 112 mg/mL, 90 mg/mL to 110 mg/mL, 90 mg/mL to 108 mg/mL, 90 mg/mL to 105 mg/mL, 90 mg/mL to 102 mg/mL, 90 mg/mL to 100 mg/mL, 90 mg/mL to 98 mg/mL, 90 mg/mL to 95 mg/mL, 100 mg/mL to 195 mg/mL, 100 mg/mL to 190 mg/mL, 100 mg/mL to 185 mg/mL, 100 mg/mL to 175 mg/mL, 100 mg/mL to 170 mg/mL, 100 mg/mL to 165 mg/mL, 100 mg/mL to 160 mg/mL, 100 mg/mL to 155 mg/mL, 100 mg/mL to 150 mg/mL, 100 mg/mL to 145 mg/mL, 100 mg/mL to 140 mg/mL, 100 mg/mL to 135 mg/mL, 100 mg/mL to 130 mg/mL, 100 mg/mL to 125 mg/mL, 100 mg/mL to 120 mg/mL, 100 mg/mL to 118 mg/mL, or 100 mg/mL to 115 mg/mL.

In some cases, the concentration of flecainide or the salt of flecainide (e.g., flecainide acetate or flecainide hydrochloride) in the pharmaceutical compositions or formulations is at least about 115 mg/mL, at least about 112 mg/mL, at least about 110 mg/mL, at least about 109 mg/mL, at least about 108 mg/mL, at least about 107 mg/mL, at least about 106 mg/mL, at least about 105 mg/mL, at least about 104 mg/mL, at least about 103 mg/mL, at least about 102 mg/mL, at least about 101 mg/mL, at least about 100 mg/mL, at least about 99 mg/mL, at least about 98 mg/mL, at least about 97 mg/mL, at least about 96 mg/mL, at least about 95 mg/mL, at least about 94 mg/mL, at least about 93 mg/mL, at least about 92 mg/mL, at least about 91 mg/mL, at least about 90 mg/mL, at least about 89 mg/mL, at least about 88 mg/mL, at least about 87 mg/mL, at least about 86 mg/mL, at least about 85 mg/mL, at least about 84 mg/mL, at least about 83 mg/mL, at least about 82 mg/mL, at least about 81 mg/mL, at least about 80 mg/mL, at least about 79 mg/mL, at least about 78 mg/mL, at least about 77 mg/mL, at least about 76 mg/mL, at least about 75 mg/mL, at least about 74 mg/mL, at least about 73 mg/mL, at least about 72 mg/mL, at least about 71 mg/mL, at least about 70 mg/mL, at least about 69 mg/mL, at least about 68 mg/mL, at least about 67 mg/mL, at least about 66 mg/mL, at least about 65 mg/mL, at least about 64 mg/mL, at least about 63 mg/mL, at least about 62 mg/mL, at least about 61 mg/mL, or at least about 60 mg/mL.

In some cases, the concentration of flecainide or the salt of flecainide (e.g., flecainide acetate or flecainide hydrochloride) in the pharmaceutical compositions or formulations is about 195 mg/mL, about 190 mg/mL, about 185 mg/mL, about 175 mg/mL, about 170 mg/mL, about 165 mg/mL, about 160 mg/mL, about 155 mg/mL, about 150 mg/mL, about 145 mg/mL, about 140 mg/mL, about 135 mg/mL, about 130 mg/mL, about 125 mg/mL, about 120 mg/mL, about 118 mg/mL, about 115 mg/mL, about 112 mg/mL, about 110 mg/mL, about 109 mg/mL, about 108 mg/mL, about 107 mg/mL, about 106 mg/mL, about 105 mg/mL, about 104 mg/mL, about 103 mg/mL, about 102 mg/mL, about 101 mg/mL, about 100 mg/mL, about 99 mg/mL, about 98 mg/mL, about 97 mg/mL, about 96 mg/mL, about 95 mg/mL, about 94 mg/mL, about 93 mg/mL, about 92 mg/mL, about 91 mg/mL, about 90 mg/mL, about 89 mg/mL, about 88 mg/mL, about 87 mg/mL, about 86 mg/mL, about 85 mg/mL, about 84 mg/mL, about 83 mg/mL, about 82 mg/mL, about 81 mg/mL, about 80 mg/mL, about 79 mg/mL, about 78 mg/mL, about 77 mg/mL, about 76 mg/mL, about 75 mg/mL, about 74 mg/mL, about 73 mg/mL, about 72 mg/mL, about 71 mg/mL, about 70 mg/mL, about 69 mg/mL, about 68 mg/mL, about 67 mg/mL, about 66 mg/mL, about 65 mg/mL, about 64 mg/mL, about 63 mg/mL, about 62 mg/mL, or about 61 mg/mL.

In some cases, the nominal concentration of flecainide or the salt of flecainide (e.g., flecainide acetate or flecainide hydrochloride) in the pharmaceutical compositions or formulations is about 125 mM to about 430 mM, such as 125 mM to 420 mM, 125 mM to 400 mM, 125 mM to 390 mM, 125 mM to 380 mM, 125 mM to 370 mM, 125 mM to 360 mM, 125 mM to 350 mM, 125 mM to 340 mM, 125 mM to 330 mM, 125 mM to 320 mM, 125 mM to 310 mM, 125 mM to 300 mM, 125 mM to 290 mM, 125 mM to 280 mM, 125 mM to 270 mM, 125 mM to 260 mM, 125 mM to 250 mM, 125 mM to 240 mM, 125 mM to 230 mM, 125 mM to 220 mM, 125 mM to 210 mM, 125 mM to 200 mM, 125 mM to 190 mM, 125 mM to 180 mM, 125 mM to 170 mM, 125 mM to 160 mM, 125 mM to 155 mM, 125 mM to 150 mM, 125 mM to 145 mM, 125 mM to 140 mM, 125 mM to 135 mM, 125 mM to 130 mM, 140 mM to 420 mM, 140 mM to 400 mM, 140 mM to 390 mM, 140 mM to 380 mM, 140 mM to 370 mM, 140 mM to 360 mM, 140 mM to 350 mM, 140 mM to 340 mM, 140 mM to 330 mM, 140 mM to 320 mM, 140 mM to 310 mM, 140 mM to 300 mM, 140 mM to 290 mM, 140 mM to 280 mM, 140 mM to 270 mM, 140 mM to 260 mM, 140 mM to 250 mM, 140 mM to 240 mM, 140 mM to 230 mM, 140 mM to 220 mM, 140 mM to 210 mM, 140 mM to 200 mM, 140 mM to 190 mM, 140 mM to 180 mM, 140 mM to 170 mM, 140 mM to 160 mM, 140 mM to 155 mM, 140 mM to 150 mM, 140 mM to 145 mM, 160 mM to 420 mM, 160 mM to 400 mM, 160 mM to 390 mM, 160 mM to 380 mM, 160 mM to 370 mM, 160 mM to 360 mM, 160 mM to 350 mM, 160 mM to 340 mM, 160 mM to 330 mM, 160 mM to 320 mM, 160 mM to 310 mM, 160 mM to 300 mM, 160 mM to 290 mM, 160 mM to 280 mM, 160 mM to 270 mM, 160 mM to 260 mM, 160 mM to 250 mM, 160 mM to 240 mM, 160 mM to 230 mM, 160 mM to 220 mM, 160 mM to 210 mM, 160 mM to 200 mM, 160 mM to 190 mM, 160 mM to 180 mM, 160 mM to 170 mM, 180 mM to 420 mM, 180 mM to 400 mM, 180 mM to 390 mM, 180 mM to 380 mM, 180 mM to 370 mM, 180 mM to 360 mM, 180 mM to 350 mM, 180 mM to 340 mM, 180 mM to 330 mM, 180 mM to 320 mM, 180 mM to 310 mM, 180 mM to 300 mM, 180 mM to 290 mM, 180 mM to 280 mM, 180 mM to 270 mM, 180 mM to 260 mM, 180 mM to 250 mM, 180 mM to 240 mM, 180 mM to 230 mM, 180 mM to 220 mM, 180 mM to 210 mM, 180 mM to 200 mM, 180 mM to 190 mM, 200 mM to 420 mM, 200 mM to 400 mM, 200 mM to 390 mM, 200 mM to 380 mM, 200 mM to 370 mM, 200 mM to 360 mM, 200 mM to 350 mM, 200 mM to 340 mM, 200 mM to 330 mM, 200 mM to 320 mM, 200 mM to 310 mM, 200 mM to 300 mM, 200 mM to 290 mM, 200 mM to 280 mM, 200 mM to 270 mM, 200 mM to 260 mM, 200 mM to 250 mM, 200 mM to 240 mM, 200 mM to 230 mM, 200 mM to 220 mM, or 200 mM to 210 mM.

In some cases, the nominal concentration of flecainide or the salt of flecainide (e.g., flecainide acetate or flecainide hydrochloride) in the pharmaceutical compositions or formulations is at least about 400 mM, at least about 390 mM, at least about 380 mM, at least about 370 mM, at least about 360 mM, at least about 350 mM, at least about 340 mM, at least about 330 mM, at least about 320 mM, at least about 310 mM, at least about 300 mM, at least about 290 mM, at least about 280 mM, at least about 270 mM, at least about 260 mM, at least about 255 mM, at least about 250 mM, at least about 245 mM, at least about 240 mM, at least about 235 mM, at least about 230 mM, at least about 225 mM, at least about 220 mM, at least about 215 mM, at least about 210 mM, at least about 200 mM, at least about 190 mM, at least about 180 mM, at least about 170 mM, at least about 160 mM, at least about 155 mM, at least about 150 mM, at least about 145 mM, at least about 140 mM, at least about 135 mM, at least about 130 mM, or at least about 125 mM.

In some cases, the concentration of flecainide or the salt of flecainide (e.g., flecainide acetate or flecainide hydrochloride) in the pharmaceutical compositions or formulations is about 420 mM, about 400 mM, about 390 mM, about 380 mM, about 370 mM, about 360 mM, about 350 mM, about 340 mM, about 330 mM, about 320 mM, about 310 mM, about 300 mM, about 290 mM, about 280 mM, about 270 mM, about 260 mM, about 255 mM, about 250 mM, about 245 mM, about 240 mM, about 235 mM, about 230 mM, about 225 mM, about 220 mM, about 215 mM, about 210 mM, about 200 mM, about 190 mM, about 180 mM, about 170 mM, about 160 mM, about 155 mM, about 150 mM, about 145 mM, about 140 mM, about 135 mM, about 130 mM, or about 125 mM.

In some aspects, also provided herein are unit doses of pharmaceutical compositions described herein for treatment of heart condition, e.g., cardiac arrhythmia, e.g., atrial arrhythmia, via oral or nasal inhalation.

In one version, a unit dose of the pharmaceutical composition provided herein includes at least about 50 mg, such as at least about 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 450 mg, 500 mg or more of flecainide or a flecainide salt, e.g., flecainide acetate or flecainide hydrochloride. A unit dose of the pharmaceutical composition provided herein can include flecainide or the flecainide salt in the range of about 50 mg to about 500 mg, such as 50 mg to 60 mg, 50 mg to 70 mg, 50 mg to 80 mg, 50 mg to 90 mg, 50 mg to 100 mg, 50 mg to 110 mg, 50 mg to 120 mg, 50 mg to 130 mg, 50 mg to 140 mg, 50 mg to 150 mg, 50 mg to 160 mg, 50 mg to 170 mg, 50 mg to 180 mg, 50 mg to 190 mg, 50 mg to 200 mg, 50 mg to 210 mg, 50 mg to 220 mg, 50 mg to 230 mg, 50 mg to 240 mg, 50 mg to 250 mg, 50 mg to 260 mg, 50 mg to 270 mg, 50 mg to 280 mg, 50 mg to 290 mg, 50 mg to 300 mg, 50 mg to 310 mg, 50 mg to 320 mg, 50 mg to 330 mg, 50 mg to 340 mg, 50 mg to 350 mg, 50 mg to 360 mg, 50 mg to 370 mg, 50 mg to 380 mg, 50 mg to 390 mg, 50 mg to 400 mg, 50 mg to 450 mg, 50 mg to 500 mg, 80 mg to 90 mg, 80 mg to 100 mg, 80 mg to 110 mg, 80 mg to 120 mg, 80 mg to 130 mg, 80 mg to 140 mg, 80 mg to 150 mg, 80 mg to 160 mg, 80 mg to 170 mg, 80 mg to 180 mg, 80 mg to 190 mg, 80 mg to 200 mg, 80 mg to 210 mg, 80 mg to 220 mg, 80 mg to 230 mg, 80 mg to 240 mg, 80 mg to 250 mg, 80 mg to 260 mg, 80 mg to 270 mg, 80 mg to 280 mg, 80 mg to 290 mg, 80 mg to 300 mg, 80 mg to 310 mg, 80 mg to 320 mg, 80 mg to 330 mg, 80 mg to 340 mg, 80 mg to 350 mg, 80 mg to 360 mg, 80 mg to 370 mg, 80 mg to 380 mg, 80 mg to 390 mg, 80 mg to 400 mg, 80 mg to 450 mg, 80 mg to 500 mg, 120 mg to 140 mg, 120 mg to 150 mg, 120 mg to 160 mg, 120 mg to 170 mg, 120 mg to 180 mg, 120 mg to 190 mg, 120 mg to 200 mg, 120 mg to 210 mg, 120 mg to 220 mg, 120 mg to 230 mg, 120 mg to 240 mg, 120 mg to 250 mg, 120 mg to 260 mg, 120 mg to 270 mg, 120 mg to 280 mg, 120 mg to 290 mg, 120 mg to 300 mg, 120 mg to 310 mg, 120 mg to 320 mg, 120 mg to 330 mg, 120 mg to 340 mg, 120 mg to 350 mg, 120 mg to 360 mg, 120 mg to 370 mg, 120 mg to 380 mg, 120 mg to 390 mg, 120 mg to 400 mg, 120 mg to 450 mg, 120 mg to 500 mg, 150 mg to 160 mg, 150 mg to 170 mg, 150 mg to 180 mg, 150 mg to 190 mg, 150 mg to 200 mg, 150 mg to 210 mg, 150 mg to 220 mg, 150 mg to 230 mg, 150 mg to 240 mg, 150 mg to 250 mg, 150 mg to 260 mg, 150 mg to 270 mg, 150 mg to 280 mg, 150 mg to 290 mg, 150 mg to 300 mg, 150 mg to 310 mg, 150 mg to 320 mg, 150 mg to 330 mg, 150 mg to 340 mg, 150 mg to 350 mg, 150 mg to 360 mg, 150 mg to 370 mg, 150 mg to 380 mg, 150 mg to 390 mg, 150 mg to 400 mg, 150 mg to 450 mg, 150 mg to 500 mg, 180 mg to 200 mg, 180 mg to 210 mg, 180 mg to 220 mg, 180 mg to 230 mg, 180 mg to 240 mg, 180 mg to 250 mg, 180 mg to 260 mg, 180 mg to 270 mg, 180 mg to 280 mg, 180 mg to 290 mg, 180 mg to 300 mg, 180 mg to 310 mg, 180 mg to 320 mg, 180 mg to 330 mg, 180 mg to 340 mg, 180 mg to 350 mg, 180 mg to 360 mg, 180 mg to 370 mg, 180 mg to 380 mg, 180 mg to 390 mg, 180 mg to 400 mg, 180 mg to 450 mg, 180 mg to 500 mg, 200 mg to 220 mg, 200 mg to 230 mg, 200 mg to 240 mg, 200 mg to 250 mg, 200 mg to 260 mg, 200 mg to 270 mg, 200 mg to 280 mg, 200 mg to 290 mg, 200 mg to 300 mg, 200 mg to 310 mg, 200 mg to 320 mg, 200 mg to 330 mg, 200 mg to 340 mg, 200 mg to 350 mg, 200 mg to 360 mg, 200 mg to 370 mg, 200 mg to 380 mg, 200 mg to 390 mg, 200 mg to 400 mg, 200 mg to 450 mg, 200 mg to 500 mg, 220 mg to 240 mg, 220 mg to 250 mg, 220 mg to 260 mg, 220 mg to 270 mg, 220 mg to 280 mg, 220 mg to 290 mg, 220 mg to 300 mg, 220 mg to 310 mg, 220 mg to 320 mg, 220 mg to 330 mg, 220 mg to 340 mg, 220 mg to 350 mg, 220 mg to 360 mg, or 340 mg to 500 mg.

In one version, a unit dose as provided herein includes flecainide or a flecainide salt of about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 450 mg, or about 500 mg.

In one aspect, provided herein are formulations for treatment of a heart condition, e.g., cardiac arrhythmia, e.g., atrial arrhythmia. The formulations can include the pharmaceutical compositions provided herein and a pharmaceutically acceptable carrier, excipient, diluent, or any other suitable component for the intended administration routes, such as oral or nasal inhalation. Examples of pharmaceutically acceptable excipients include, but are not limited to, lipids, metal ions, surfactants, amino acids, carbohydrates, buffers, salts, polymers, and the like, and combinations thereof.

The pharmaceutical formulation according to one or more embodiments of the disclosure may comprise a salt of flecainide and, optionally, one or more other active ingredients and, optionally, one or more pharmaceutically acceptable excipients. For example, the pharmaceutical formulation can comprise particles of the flecainide salt with no other ingredients added (neat particles), may comprise neat particles of antiarrhythmic pharmaceutical agent together with other particles, and/or may comprise particles comprising antiarrhythmic pharmaceutical agent and one or more active ingredients and/or one or more pharmaceutically acceptable excipients.

In one aspect, also provided herein are kits for treatment of heart conditions via inhalation. The kits can include one or more pharmaceutical agents, for instance, a salt of flecainide, or some additional active agent(s) as described herein. In some cases, the kits include container for the pharmaceutical agents or compositions. In some cases, unit doses of the pharmaceutical agents as discussed above are provided in the kits. In some cases, the kits also include containers/receptacles for containing the pharmaceutical agents.

In some cases, the kits include separate containers/receptacles for containing the pharmaceutical composition as described herein. In some cases, the kits include an aerosolization device for forming an aerosol of the pharmaceutical compositions. The aerosolization device can be any device as provided herein, and in some cases, used for inhalation of the pharmaceutical compositions. In some cases, the kits include nasal spray device as provided herein. In some cases, the pharmaceutical composition(s) is/are present in aerosol form in the kits. In some other cases, the kits include a single container for containing the pharmaceutical composition. The kits can further include instructions for methods of using the kit. The instructions can be presented in the form of a data sheet, a manual, in a piece of paper, printed on one or more containers or devices of the kit. Alternatively, the instructions can be provided in electronic form, for instance, available in a disc or online with a weblink available from the kit. The instructions for use of the kit can comprise instructions for use of the pharmaceutical composition and the aerosolization device (e.g., a nebulizer) to treat any applicable indication, e.g., a heart condition, e.g., cardiac arrhythmia, e.g., atrial arrhythmia. The instructions for use of the kit can comprise instructions for use of the pharmaceutical composition and the aerosolization device (e.g., a nebulizer) to treat atrial fibrillation. In some cases, the kits include a nose clip. A nose clip can be used to hinder passage of air through a nose of a subject during inhalation and increase the proportion of a total inhaled volume that is the aerosol issued by the nebulizer.

Examples of carbohydrates include, but are not limited to, monosaccharides, disaccharides, and polysaccharides. For example, monosaccharides such as dextrose (anhydrous and monohydrate), galactose, mannitol, D-mannose, sorbitol, sorbose and the like; disaccharides such as lactose, maltose, sucrose, trehalose, and the like; trisaccharides such as raffinose and the like; and other carbohydrates such as starches (hydroxyethylstarch), and maltodextrins.

Non-limiting examples of lipids include phospholipids, glycolipids, ganglioside GM1, sphingomyelin, phosphatidic acid, cardiolipin; lipids bearing polymer chains such as polyethylene glycol, chitin, hyaluronic acid, or polyvinylpyrrolidone; lipids bearing sulfonated mono-, di-, and polysaccharides; fatty acids such as palmitic acid, stearic acid, and oleic acid; cholesterol, cholesterol esters, and cholesterol hemisuccinate.

In some cases, the phospholipid comprises a saturated phospholipid, such as one or more phosphatidylcholines. Exemplary acyl chain lengths are 16:0 and 18:0 (e.g., palmitoyl and stearoyl). The phospholipid content can be determined by the active agent activity, the mode of delivery, and other factors.

Phospholipids from both natural and synthetic sources can be used in varying amounts. When phospholipids are present, the amount is typically sufficient to coat the active agent(s) with at least a single molecular layer of phospholipid. In general, the phospholipid content ranges from about 5 wt % to about 99.9 wt %, such as about 20 wt % to about 80 wt %.

Generally, compatible phospholipids can comprise those that have a gel to liquid crystal phase transition greater than about 40° C., such as greater than about 60° C., or greater than about 80° C. The incorporated phospholipids can be relatively long chain (e.g., C₁₆-C₂₂) saturated lipids. Exemplary phospholipids useful in the present disclosure include, but are not limited to, phosphoglycerides such as dipalmitoylphosphatidylcholine, distearoylphosphatidylcholine, diarachidoylphosphatidylcholine, dibehenoylphosphatidylcholine, diphosphatidyl glycerols, short-chain phosphatidylcholines, hydrogenated phosphatidylcholine, E-100-3 (available from Lipoid KG, Ludwigshafen, Germany), long-chain saturated phosphatidylethanolamines, long-chain saturated phosphatidylserines, long-chain saturated phosphatidylglycerols, long-chain saturated phosphatidylinositols, phosphatidic acid, phosphatidylinositol, and sphingomyelin.

Examples of metal ions include, but are not limited to, divalent cations, including calcium, magnesium, zinc, iron, and the like. For instance, when phospholipids are used, the pharmaceutical composition can also comprise a polyvalent cation, as disclosed in WO 01/85136 and WO 01/85137, which are incorporated herein by reference in their entireties. The polyvalent cation can be present in an amount effective to increase the melting temperature (Tm) of the phospholipid such that the pharmaceutical composition exhibits a Im which is greater than its storage temperature (Tₘ) by at least about 20° C., such as at least about 40° C. The molar ratio of polyvalent cation to phospholipid can be at least about 0.05:1, such as about 0.05:1 to about 2.0:1 or about 0.25:1 to about 1.0:1. An example of the molar ratio of polyvalent cation: phospholipid is about 0.50:1. When the polyvalent cation is calcium, it can be in the form of calcium chloride. Although metal ion, such as calcium, is often included with phospholipid, none is required.

The pharmaceutical composition can include one or more surfactants. For instance, one or more surfactants can be in the liquid phase with one or more being associated with solid particles or particles of the composition. By “associated with” it is meant that the pharmaceutical compositions can incorporate, adsorb, absorb, be coated with, or be formed by the surfactant. Surfactants include, but are not limited to, fluorinated and nonfluorinated compounds, such as saturated and unsaturated lipids, nonionic detergents, nonionic block copolymers, ionic surfactants, and combinations thereof. It should be emphasized that, in addition to the aforementioned surfactants, suitable fluorinated surfactants are compatible with the teachings herein and can be used to provide the desired preparations.

Examples of nonionic detergents include, but are not limited to, sorbitan esters including sorbitan trioleate (Span™ 85), sorbitan sesquioleate, sorbitan monooleate, sorbitan monolaurate, polyoxyethylene (20) sorbitan monolaurate, and polyoxyethylene (20) sorbitan monooleate, oleyl polyoxyethylene (2) ether, stearyl polyoxyethylene (2) ether, lauryl polyoxyethylene (4) ether, glycerol esters, and sucrose esters. Other suitable nonionic detergents can be easily identified using Mccutcheon's Emulsifiers and Detergents (McPublishing Co., Glen Rock, N.J.), which is incorporated herein by reference in its entirety.

Examples of block copolymers include, but are not limited to, diblock and triblock copolymers of polyoxyethylene and polyoxypropylene, including poloxamer 188 (Pluronic™ F-68), poloxamer 407 (Pluronic™ F-127), and poloxamer 338. Examples of ionic surfactants include, but are not limited to, sodium sulfosuccinate, and fatty acid soaps. Examples of amino acids include, but are not limited to hydrophobic amino acids. Use of amino acids as pharmaceutically acceptable excipients is known in the art as disclosed in WO 95/31479, WO 96/32096, and WO 96/32149, which are incorporated herein by reference in their entireties.

Examples of buffers include, but are not limited to, acetate, tris, or citrate. Examples of acids include, but are not limited to, carboxylic acids. Examples of salts include, but are not limited to, sodium chloride, salts of carboxylic acids, (e.g., sodium citrate, sodium ascorbate, magnesium gluconate, sodium gluconate, tromethamine hydrochloride, etc.), ammonium carbonate, ammonium acetate, ammonium chloride, and the like. Examples of organic solids include, but are not limited to, camphor, and the like. The pharmaceutical composition of one or more embodiments of the present disclosure can also include a biocompatible, such as biodegradable polymer, copolymer, or blend or other combination thereof. In this respect useful polymers comprise polylactides, polylactide-glycolides, cyclodextrins, polyacrylates, methylcellulose, carboxymethylcellulose, polyvinyl alcohols, polyanhydrides, polylactams, polyvinyl pyrrolidones, polysaccharides (dextrans, starches, chitin, chitosan, etc.), hyaluronic acid, proteins, (albumin, collagen, gelatin, etc.). Those skilled in the art will appreciate that, by selecting the appropriate polymers, the delivery efficiency of the composition and/or the stability of the dispersions can be tailored to optimize the effectiveness of the antiarrhythmic pharmaceutical agent(s).

For solutions, the compositions can include one or more osmolality adjuster, such as sodium chloride. For instance, sodium chloride can be added to solutions to adjust the osmolality of the solution. In one or more embodiments, an aqueous composition consists essentially of the antiarrhythmic pharmaceutical agent, the osmolality adjuster, and water.

Solutions can also comprise a buffer or a pH adjusting agent, typically a salt prepared from an organic acid or base. Representative buffers comprise organic acid salts of citric acid, lactic acid, ascorbic acid, gluconic acid, carbonic acid, tartaric acid, succinic acid, acetic acid, or phthalic acid, Tris, tromethamine hydrochloride, or phosphate buffers. Thus, the buffers can include citrates, phosphates, phthalates, and lactates.

Besides the above mentioned pharmaceutically acceptable excipients, it can be desirable to add other pharmaceutically acceptable excipients to the pharmaceutical composition to improve particle rigidity, production yield, emitted dose and deposition, shelf-life, and patient acceptance. Such optional pharmaceutically acceptable excipients include, but are not limited to: coloring agents, taste masking agents, buffers, hygroscopic agents, antioxidants, and chemical stabilizers. Further, various pharmaceutically acceptable excipients can be used to provide structure and form to the particle compositions (e.g., latex particles). In this regard, it will be appreciated that the rigidifying components can be removed using a post-production technique such as selective solvent extraction.

The pharmaceutical compositions of one or more embodiments of the present disclosure can lack taste. In this regard, although taste masking agents are optionally included within the composition, the compositions in some embodiments do not include a taste masking agent other than a cyclodextrin and lack taste even without a taste masking agent.

The pharmaceutical compositions can also include mixtures of pharmaceutically acceptable excipients. For instance, mixtures of carbohydrates and amino acids are within the scope of the present disclosure.

The compositions of one or more embodiments of the present disclosure can take various forms, such as solutions, dry powders, reconstituted powders, suspensions, or dispersions comprising a non-aqueous phase, such as propellants (e.g., chlorofluorocarbon, hydrofluoroalkane).

The solutions of the present disclosure are typically clear. In this regard, many of the antiarrhythmic pharmaceutical agents of the present disclosure are water soluble.

In some embodiments, the isotonicity of the solution ranges from isotonic to physiologic isotonicity. Physiologic isotonicity is the isotonicity of physiological fluids.

In some versions, the pharmaceutical composition is a nebulized aerosol and comprises liquid droplets having a mass median diameter less than about 20 μm, such as less than about 10 μm, less than about 7 μm, or less than about 5 μm. The droplets can have a mass median aerodynamic diameter ranging from about 1 μm to about 6 μm, such as about 1.5 μm to about 5 μm, or about 2 μm to about 4 μm. If the droplets are too large, a larger percentage of the particles cannot reach the lungs. If the droplets are too small, a larger percentage of the droplets can be exhaled.

Unit doses of the pharmaceutical compositions can be placed in a container. Examples of containers include, but are not limited to, syringes, capsules, blow fill seal, blisters, vials, ampoules, cartridges, or container closure systems made of metal, polymer (e.g., plastic, elastomer), glass, or the like. For instance, the vial can be a colorless Type I borosilicate glass ISO 4R 6 mL vial with a chlorobutyl rubber siliconized stopper, and flip-off type aluminum cap with colored plastic cover. In some embodiments, the vial can be a colorless Type I borosilicate glass ISO 6R 10 mL vial with a chlorobutyl rubber siliconized stopper, and flip-off type aluminum cap with colored plastic cover.

The container can be inserted into an aerosolization device. The container can be of a suitable shape, size, and material to contain the pharmaceutical composition and to provide the pharmaceutical composition in a usable condition. For example, the capsule or blister can comprise a wall which comprises a material that does not adversely react with the pharmaceutical composition. In addition, the wall can comprise a material that allows the capsule to be opened to allow the pharmaceutical composition to be aerosolized. In one version, the wall comprises one or more of gelatin, hydroxypropyl methylcellulose (HPMC), polyethyleneglycol-compounded HPMC, hydroxyproplycellulose, agar, aluminum foil, or the like. In one version, the capsule can comprise telescopically adjoining sections, as described for example in U.S. Pat. No. 4,247,066 which is incorporated herein by reference in its entirety. The size of the capsule can be selected to adequately contain the dose of the pharmaceutical composition. The sizes generally range from size 5 to size 000 with the outer diameters ranging from about 4.91 mm to 9.97 mm, the heights ranging from about 11.10 mm to about 26.14 mm, and the volumes ranging from about 0.10 ml to about 1.37 mL, respectively. Suitable capsules are available commercially from, for example, Shionogi Qualicaps Co. in Nara, Japan and Capsugel in Greenwood, S.C. After filling, a top portion can be placed over the bottom portion to form a capsule shape and to contain the powder within the capsule, as described in U.S. Pat. Nos. 4,846,876 and 6,357,490, and in WO 00/07572, which are incorporated herein by reference in their entireties. After the top portion is placed over the bottom portion, the capsule can optionally be banded.

For solutions, the amount of the composition in the unit dose can range from about 0.5 mL to about 15 mL, such as 1 mL to 15 mL, 2 mL to 15 mL, 3 mL to 15 mL, 4 mL to 15 mL, 5 mL to 15 mL, 6 mL to 15 mL, 7 mL to 15 mL, 8 mL to 15 mL, 9 mL to 15 mL, 10 mL to 15 mL, 11 mL to 15 mL, 12 mL to 15 mL, 10 mL to 15 mL, 14 mL to 15 mL, 1 mL to 13 mL, 2 mL to 13 mL, 3 mL to 13 mL, 4 mL to 13 mL, 5 mL to 13 mL, 6 mL to 13 mL, 7 mL to 13 mL, 8 mL to 13 mL, 9 mL to 13 mL, 10 mL to 13 mL, 11 mL to 13 mL, 12 mL to 13 mL, 1 mL to 10 mL, 2 mL to 10 mL, 3 mL to 10 mL, 4 mL to 10 mL, 5 mL to 10 mL, 6 mL to 10 mL, 7 mL to 10 mL, 8 mL to 10 mL, 9 mL to 10 mL, 1 mL to 8 mL, 2 mL to 8 mL, 3 mL to 8 mL, 4 mL to 8 mL, 5 mL to 8 mL, 6 mL to 8 mL, 7 mL to 8 mL, 1 mL to 5 mL, 2 mL to 5 mL, 3 mL to 5 mL, 4 mL to 5 mL, or 1 mL to 3 mL. In some embodiments, the amount of the composition in a unit dose is about 1 mL, about 2 mL, about 3 mL, about 4 mL, about 5 mL, about 6 mL, about 7 mL, about 8 mL, about 9 mL, about 10 mL, about 11 mL, about 12 mL, about 10 mL, about 14 mL, or about 15 mL. In some embodiments, the amount of the composition in a unit dose is at most about 2 mL, at most about 3 mL, at most about 4 mL, at most about 5 mL, at most about 6 mL, at most about 7 mL, at most about 8 mL, at most about 9 mL, at most about 10 mL, at most about 11 mL, at most about 12 mL, at most about 10 mL, at most about 14 mL, or at most about 15 mL.

The compositions of the present disclosure can be made by any of the various methods and techniques known and available to those skilled in the art.

For instance, a solution of antiarrhythmic pharmaceutical agent can be made using the following procedure. Typically, manufacturing equipment is sterilized before use. A portion of the final volume, e.g., 70%, of solvent, e.g., water for injection, can be added into a suitable container. Some or all of other additional pharmaceutically acceptable carrier or excipient, solubilizer, or other additional ingredients of the pharmaceutical composition (e.g., cyclodextrin, e.g., HPβCD; e.g., acids, e.g., acetic acid, hydrochloric acid, nitric acid, or citric acid; e.g., saccharin, e.g., saccharin sodium) can be added either before or after addition of the antiarrhythmic agent, e.g., the flecainide salt, e.g., flecainide acetate. Antiarrhythmic pharmaceutical agent, e.g., a salt of flecainide can then be added. The antiarrhythmic pharmaceutical agent can be mixed until dissolved. Additional solvent can be added to make up the final batch volume. The batch can be filtered, e.g., through a 0.2 μm filter into a sterilized receiving vessel. Filling components can be sterilized before use in filling the batch into vials, e.g., 10 ml vials.

As an example, the above-noted sterilizing can include the following. A 5 liter type 1 glass bottle and lid can be placed in an autoclave bag and sterilized at elevated temperature, e.g., 121° C. for 15 minutes, using an autoclave. Similarly, vials can be placed into suitable racks, inserted into an autoclave bag, and sterilized at elevated temperature, e.g., 121° C. for 15 minutes, using an autoclave. Also, similarly, stoppers can be placed in an autoclave bag and sterilized at elevated temperature, e.g., 121° C. for 15 minutes, using an autoclave. Before sterilization, sterilizing filters can be attached to tubing, e.g., a 2 mm length of 7 mm×13 mm silicone tubing. A filling line can be prepared by placed in an autoclave bag and sterilized at elevated temperature, e.g., 121° C. for 15 minutes, using an autoclave.

The above-noted filtration can involve filtration into a laminar flow work area. The receiving bottle and filters can be set up in the laminar flow work area.

The above-noted filling can also be conducted under laminar flow protection. The filling line can be unwrapped and placed into the receiving bottle. The sterilized vials and stoppers can be unwrapped under laminar flow protection. Each vial can be filled, e.g., to a target fill of 5 g, and stoppered. A flip off collar can be applied to each vial. The sealed vials can be inspected for vial leakage, correct overseals, and cracks.

The pharmaceutical composition according to one or more embodiments of the disclosure may, if desired, contain a combination of antiarrhythmic pharmaceutical agent (e.g., flecainide salt) and one or more additional active agents. Examples of additional active agents include, but are not limited to, agents that may be delivered through the lungs.

Additional active agents may comprise, for example, hypnotics and sedatives, psychic energizers, tranquilizers, respiratory drugs, anticonvulsants, muscle relaxants, antiparkinson agents (dopamine antagonists), analgesics, anti-inflammatoires, antianxiety drugs (anxiolytics), appetite suppressants, antimigraine agents, muscle contractants, additional anti-infectives (antivirals, antifungals, vaccines) antiarthritics, antimalarials, antiemetics, antiepileptics, cytokines, growth factors, anti-cancer agents, antithrombotic agents, antihypertensives, cardiovascular drugs, antiarrhythmics, antioxidants, anti-asthma agents, hormonal agents including contraceptives, sympathomimetics, diuretics, lipid regulating agents, antiandrogenic agents, antiparasitic, anticoagulants, neoplastics, antineoplastics, hypoglycemics, nutritional agents and supplements, growth supplements, antienteritis agents, vaccines, antibodies, diagnostic agents, and contrasting agents. The additional active agent, when administered by inhalation, may act locally or systemically.

The additional active agent may fall into one of a number of structural classes, including but not limited to small molecules, peptides, polypeptides, proteins, polysaccharides, steroids, proteins capable of eliciting physiological effects, nucleotides, oligonucleotides, polynucleotides, fats, electrolytes, and the like.

Examples of additional active agents suitable for use in this disclosure include but are not limited to one or more of calcitonin, amphotericin B, erythropoietin (EPO), Factor VIII, Factor IX, ceredase, cerezyme, cyclosporin, granulocyte colony stimulating factor (GCSF), thrombopoietin (TPO), alpha-1 proteinase inhibitor, elcatonin, granulocyte macrophage colony stimulating factor (GMCSF), growth hormone, human growth hormone (HGH), growth hormone releasing hormone (GHRH), heparin, low molecular weight heparin (LMWH), interferon alpha, interferon beta, interferon gamma, interleukin-1 receptor, interleukin-2, interleukin-1 receptor antagonist, interleukin-3, interleukin-4, interleukin-6, luteinizing hormone releasing hormone (LHRH), factor IX, insulin, pro-insulin, insulin analogues (e.g., mono-acylated insulin as described in U.S. Pat. No. 5,922,675, which is incorporated herein by reference in its entirety), amylin, C-peptide, somatostatin, somatostatin analogs including octreotide, vasopressin, follicle stimulating hormone (FSH), insulin-like growth factor (IGF), insulintropin, macrophage colony stimulating factor (M-CSF), nerve growth factor (NGF), tissue growth factors, keratinocyte growth factor (KGF), glial growth factor (GGF), tumor necrosis factor (TNF), endothelial growth factors, parathyroid hormone (PTH), glucagon-like peptide thymosin alpha 1, IIb/Ila inhibitor, alpha-1 antitrypsin, phosphodiesterase (PDE) compounds, VLA-4 inhibitors, bisphosponates, respiratory syncytial virus antibody, cystic fibrosis transmembrane regulator (CFFR) gene, deoxyribonuclease (DNase), bactericidal/permeability increasing protein (BPI), anti-CMV antibody, 13-cis retinoic acid, oleandomycin, troleandomycin, roxithromycin, clarithromycin, davercin, azithromycin, flurithromycin, dirithromycin, josamycin, spiromycin, midecamycin, leucomycin, miocamycin, rokitamycin, andazithromycin, and swinolide A; fluoroquinolones such as ciprofloxacin, ofloxacin, levofloxacin, trovafloxacin, alatrofloxacin, moxifloxicin, norfloxacin, enoxacin, grepafloxacin, gatifloxacin, lomefloxacin, sparfloxacin, temafloxacin, pefloxacin, amifloxacin, fleroxacin, tosufloxacin, prulifloxacin, irloxacin, pazufloxacin, clinafloxacin, and sitafloxacin, teicoplanin, rampolanin, mideplanin, colistin, daptomycin, gramicidin, colistimethate, polymixins such as polymixin B, capreomycin, bacitracin, penems; penicillins including penicilinase-sensitive agents like penicillin G, penicillin V, penicillinase-resistant agents like methicillin, oxacillin, cloxacillin, dicloxacillin, floxacillin, nafcillin; gram negative microorganism active agents like ampicillin, amoxicillin, and hetacillin, cillin, and galampicillin; antipseudomonal penicillins like carbenicillin, ticarcillin, azlocillin, mezlocillin, and piperacillin; cephalosporins like cefpodoxime, cefprozil, ceftbuten, ceftizoxime, ceftriaxone, cephalothin, cephapirin, cephalexin, cephradrine, cefoxitin, cefamandole, cefazolin, cephaloridine, cefaclor, cefadroxil, cephaloglycin, cefuroxime, ceforanide, cefotaxime, cefatrizine, cephacetrile, cefepime, cefixime, cefonicid, cefoperazone, cefotetan, cefinetazole, ceftazidime, loracarbef, and moxalactam, monobactams like aztreonam; and carbapenems such as imipenem, meropenem, pentamidine isethiouate, lidocaine, metaproterenol sulfate, beclomethasone diprepionate, triamcinolone acetamide, budesonide acetonide, fluticasone, ipratropium bromide, flunisolide, cromolyn sodium, ergotamine tartrate and where applicable, analogues, agonists, antagonists, inhibitors, and pharmaceutically acceptable salt forms of the above. In reference to peptides and proteins, the disclosure is intended to encompass synthetic, native, glycosylated, unglycosylated, pegylated forms, and biologically active fragments, derivatives, and analogs thereof.

Additional active agents for use in the disclosure can further include nucleic acids, as bare nucleic acid molecules, vectors, associated viral particles, plasmid DNA or RNA or other nucleic acid constructions of a type suitable for transfection or transformation of cells, e.g., suitable for gene therapy including antisense. Further, an active agent may comprise live attenuated or killed viruses suitable for use as vaccines. Other useful drugs include those listed within the Physician's Desk Reference (most recent edition), which is incorporated herein by reference in its entirety.

When a combination of active agents is used, the agents may be provided in combination in a single species of pharmaceutical composition or individually in separate species of pharmaceutical compositions.

In some embodiments, the present disclosure provides a kit comprising a first dose and a second dose of a pharmaceutical composition formulated for oral inhalation, wherein:
(a) said pharmaceutical composition comprises a class I antiarrhythmic agent or a pharmaceutically acceptable salt thereof;
(b) said second dose comprises said class I antiarrhythmic agent or a pharmaceutically acceptable salt thereof in an amount that is equivalent to about 40% to about 80% (w/w) of said first dose;
(c) said first dose is provided in a first vessel, and said second dose is provided in a second vessel.

In some embodiments, said second dose comprises said salt of flecainide in an amount that is equivalent to about 40% to about 80% (w/w) of said first dose. In some embodiments, said second dose comprises said salt of flecainide in an amount that is equivalent to said first dose. In some embodiments, said second dose comprises said salt of flecainide in an amount that is equivalent to about 40% to about 60% (w/w) of said first dose. In some embodiments, said second dose comprises said salt of flecainide in an amount that is equivalent to about 50% (w/w) of said first dose.

In some embodiments, said second dose comprises said salt of flecainide in an amount that is equivalent to about 40% to about 80% (w/w) of said first dose. In some embodiments, said second dose comprises said salt of flecainide in an amount that is equivalent to about 65% to about 85% (w/w) of said first dose. In some embodiments, said second dose comprises said salt of flecainide in an amount that is equivalent to said first dose. In some embodiments, said second dose comprises said salt of flecainide in an amount that is equivalent to about 15% to about 35% (w/w) of said first dose. In some embodiments, said second dose comprises said salt of flecainide in an amount that is equivalent to about 40% to about 60% (w/w) of said first dose. In some embodiments, said second dose comprises said salt of flecainide in an amount that is equivalent to about 40% (w/w) of said first dose. In some embodiments, said second dose comprises said salt of flecainide in an amount that is equivalent to about 50% (w/w) of said first dose. In some embodiments, said second dose comprises said salt of flecainide in an amount that is equivalent to about 60% (w/w) of said first dose. In some embodiments, said second dose comprises said salt of flecainide in an amount that is equivalent to about 70% (w/w) of said first dose. In some embodiments, said second dose comprises said salt of flecainide in an amount that is equivalent to about 25% (w/w) of said first dose. In some embodiments, said second dose comprises said salt of flecainide in an amount that is equivalent to about 75% (w/w) of said first dose.

In some embodiments, said first dose comprises from about 100 mg to about 250 mg said salt of flecainide. In some embodiments, said first dose comprises from about 100 mg to about 140 mg said salt of flecainide. In some embodiments, said first dose comprises from about 20 mg to about 40 mg said salt of flecainide. In some embodiments, said first dose comprises from about 50 mg to about 70 mg said salt of flecainide. In some embodiments, said first dose comprises from about 80 mg to about 100 mg said salt of flecainide. In some embodiments, said first dose comprises about 120 mg said salt of flecainide. In some embodiments, said first dose comprises about 90 mg said salt of flecainide. In some embodiments, said first dose comprises about 60 mg said salt of flecainide. In some embodiments, said first dose comprises about 30 mg said salt of flecainide.

In some embodiments, said second dose comprises from about 30 mg to about 90 mg said salt of flecainide. In some embodiments, said second dose comprises from about 50 mg to about 70 mg said salt of flecainide. In some embodiments, said second dose comprises from about 20 mg to about 40 mg said salt of flecainide. In some embodiments, said second dose comprises from about 50 mg to about 70 mg said salt of flecainide. In some embodiments, said second dose comprises from about 80 mg to about 100 mg said salt of flecainide.

In some embodiments, administration of said first dose results in a peak plasma concentration (Cₘₐₓ) of said salt of flecainide in said subject that is at least 200 ng/mL. In some embodiments, said Cₘₐₓ is between about 250 ng/ml and about 1000 ng/ml. In some embodiments, said Cₘₐₓ is between about 300 ng/ml and about 700 ng/ml.

In some embodiments, said kit further comprises a nebulizer. In some embodiments, said nebulizer is a breath-actuated nebulizer. In some embodiments, said nebulizer is a jet nebulizer. In some embodiments, said nebulizer is a vibrating mesh nebulizer.

In some embodiments, the kit further comprises instructions for administration of said first dose and said second dose in accordance with a method described herein.

In some embodiments, the present disclosure provides a kit described herein, wherein said kit is for use in treating a patient suffering from atrial fibrillation, wherein said patient has a qualifying BMI, wherein said qualifying BMI is a BMI that is at least 23 kg/m². In some embodiments, said qualifying BMI is a BMI that is at least 25 kg/m². In some embodiments, said qualifying BMI is a BMI that is at least 27 kg/m². In some embodiments, said qualifying BMI is a BMI that is at least 29 kg/m². In some embodiments, said qualifying BMI is a BMI that is at least 30 kg/m². In some embodiments, said qualifying BMI is a BMI that is from about 23 kg/m² to about 40 kg/m². In some embodiments, said qualifying BMI is a BMI that is from about 23 kg/m² to about 37 kg/m². In some embodiments, said qualifying BMI is a BMI that is from about 23 kg/m² to about 35 kg/m². In some embodiments, said qualifying BMI is a BMI that is from about 25 kg/m² to about 40 kg/m². In some embodiments, said qualifying BMI is a BMI that is from about 25 kg/m² to about 37 kg/m². In some embodiments, said qualifying BMI is a BMI that is from about 25 kg/m² to about 35 kg/m². In some embodiments, said qualifying BMI is a BMI from about 25 kg/m² to about 30 kg/m². In some embodiments, said qualifying BMI is a BMI from about 30 kg/m² to about 40 kg/m². In some embodiments, said qualifying BMI is a BMI from about 25 kg/m² to about 40 kg/m².

In some embodiments, the present disclosure provides a pharmaceutical composition comprising a therapeutically effective amount of an antiarrhythmic agent for use in treating a patient suffering from a heart condition, wherein said patient has a body mass index (BMI) of at most 40 kg/m², wherein said pharmaceutical composition is administered to said subject via inhalation.

In some embodiments, the present disclosure provides an inhalable pharmaceutical composition comprising a therapeutically effective amount of an antiarrhythmic agent for use in treating a patient suffering from a heart condition, wherein said patient has a body mass index (BMI) of at most 40 kg/m². In some embodiments, said patient has a body mass index (BMI) of at most 39 kg/m². In some embodiments, said patient has a body mass index (BMI) of at most 38 kg/m². In some embodiments, said patient has a body mass index (BMI) of at most 37 kg/m². In some embodiments, said patient has a body mass index (BMI) of at most 36 kg/m². In some embodiments, said patient has a body mass index (BMI) of at most 35 kg/m².

In some embodiments, the present disclosure provides an aerosolized solution comprising a therapeutically effective amount of an antiarrhythmic agent for use in treating a patient suffering from a heart condition, wherein said patient has a body mass index (BMI) of at most 40 kg/m². In some embodiments, said patient has a body mass index (BMI) of at most 38 kg/m². In some embodiments, said patient has a body mass index (BMI) of at most 37 kg/m². In some embodiments, said patient has a body mass index (BMI) of at most 36 kg/m². In some embodiments, said patient has a body mass index (BMI) of at most 35 kg/m².

In some embodiments, the present disclosure provides a method of treating a heart condition, comprising:
identifying a human subject that has experienced or is experiencing said heart condition, and that has a body mass index (BMI) of no more than 40 kg/m²; and
administering to said subject via oral inhalation a pharmaceutical composition that comprises a therapeutically effective amount of an antiarrhythmic agent.

In some embodiments, said identifying comprises obtaining a measured body mass of said subject. In some embodiments, said identifying comprises measuring body mass of said subject to obtain said measured body mass of said subject. In some embodiments, said identifying comprises obtaining a measured body mass and a measured height of said subject. In some embodiments, said identifying further comprises measuring said body mass and height of said subject to obtain said measured body mass and said measured height. In some embodiments, said identifying further comprises calculating BMI of said subject based on said measured body mass and said measured height.

In some embodiments, the present disclosure provides a method of treatment of a heart condition, comprising:
administering to a human subject via inhalation a pharmaceutical composition that comprises a therapeutically effective amount of an antiarrhythmic agent, wherein said subject has been prescribed with said pharmaceutical composition based, at least in part, on that: (a) said subject has experienced or is experiencing said heart condition, and (b) said subject has a body mass index (BMI) of at most 40 kg/m².

In some embodiments, said subject is determined to have a body mass of at most 100 kg, 95 kg, 90 kg, 85 kg, or 80 kg. In some embodiments, said subject is determined to have a body mass of at most 90 kg, 89 kg, 88 kg, 87 kg, 86 kg, 85 kg, 84 kg, 83 kg, 82 kg, 81 kg, or 80 kg. In some embodiments, said subject is determined to have a body mass of less than 90 kg, 89 kg, 88 kg, 87 kg, 86 kg, 85 kg, 84 kg, 83 kg, 82 kg, 81 kg, or 80 kg.

In some embodiments, said subject is determined to have a BMI of at most 37 kg/m², 35 kg/m², 33 kg/m², 31 kg/m², 30 kg/m², 29 kg/m², 28 kg/m², 27 kg/m², 26 kg/m², or 25 kg/m². In some embodiments, said subject is determined to have a BMI of at most 32 kg/m², 31.5 kg/m², 31 kg/m², 30.5 kg/m², 30 kg/m², 29.5 kg/m², 29 kg/m², 28.5 kg/m², 28 kg/m², 27.5 kg/m², 27 kg/m², 26.5 kg/m², 26 kg/m², 25.5 kg/m², or 25 kg/m². In some embodiments, said patient has a body mass index (BMI) of at most 38 kg/m². In some embodiments, said patient has a body mass index (BMI) of at most 37 kg/m². In some embodiments, said patient has a body mass index (BMI) of at most 36 kg/m². In some embodiments, said patient has a body mass index (BMI) of at most 35 kg/m².

In some embodiments, said subject is determined to have a BMI of less than 32 kg/m², 31.5 kg/m², 31 kg/m², 30.5 kg/m², 30 kg/m², 29.5 kg/m², 29 kg/m², 28.5 kg/m², 28 kg/m², 27.5 kg/m², 27 kg/m², 26.5 kg/m², 26 kg/m², 25.5 kg/m², or 25 kg/m².

In some embodiments, the present disclosure provides a method of treating a human subject suffering from a heart condition, comprising:
obtaining a measured body mass index (BMI) of said subject; and
then administering to said subject via inhalation a pharmaceutical composition if said measured BMI is at most 40 kg/m²,
wherein said pharmaceutical composition comprises a therapeutically effective amount of an antiarrhythmic agent.

In some embodiments, said obtaining comprises measuring a body mass of said subject. In some embodiments, said obtaining comprises obtaining said measured body mass and a measured height of said subject. In some embodiments, said obtaining further comprises measuring weight and height of said subject to obtain said measured body mass and said measured height. In some embodiments, said obtaining said measurement further comprises calculating said measured BMI based on said measured body mass and said measured height.

In some embodiments, the method comprises administering to said subject via inhalation said pharmaceutical composition if said measured BMI is at most 37 kg/m², 35 kg/m², 33 kg/m², 31 kg/m², 30 kg/m², 29 kg/m², 28 kg/m², 27 kg/m², 26 kg/m², or 25 kg/m². In some embodiments, the method comprises administering to said subject via inhalation said pharmaceutical composition if said measured BMI is at most 32 kg/m², 31.5 kg/m², 31 kg/m², 30.5 kg/m², 30 kg/m², 29.5 kg/m², 29 kg/m², 28.5 kg/m², 28 kg/m², 27.5 kg/m², 27 kg/m², 26.5 kg/m², 26 kg/m², 25.5 kg/m², or 25 kg/m². In some embodiments, the method comprises administering to said subject via inhalation said pharmaceutical composition if said measured BMI is less than 32 kg/m², 31.5 kg/m², 31 kg/m², 30.5 kg/m², 30 kg/m², 29.5 kg/m², 29 kg/m², 28.5 kg/m², 28 kg/m², 27.5 kg/m², 27 kg/m², 26.5 kg/m², 26 kg/m², 25.5 kg/m², or 25 kg/m². In some embodiments, the method comprises administering to said subject via inhalation said pharmaceutical composition if said measured BMI is at most 35 kg/m². In some embodiments, said measured height, or said BMI is measured at the time of treating.

In some embodiments, said subject has a systolic blood pressure that is greater than about 90 mmHg at the time of treating. In some embodiments, said subject has a systolic blood pressure that is from about 100 mmHg to about 160 mmHg at the time of treating.

In some embodiments, said subject has a ventricular rate that is no more than 170 BPM at the time of treating. In some embodiments, said subject has a ventricular rate that is no more than 160 BPM at the time of treating. In some embodiments, said subject has a ventricular rate that is no more than 155 BPM at the time of treating. In some embodiments, said subject has a ventricular rate that is from about 40 BPM to about 155 BPM at the time of treating. In some embodiments, said subject has a ventricular rate that is from about 50 BPM to about 155 BPM at the time of treating. In some embodiments, said subject has a ventricular rate that is from about 60 BPM to about 155 BPM at the time of treating. In some embodiments, said subject has a ventricular rate that is from about 70 BPM to about 155 BPM at the time of treating. In some embodiments, said subject has a ventricular rate that is from about 80 BPM to about 155 BPM at the time of treating.

In one aspect, the present disclosure provides a pharmaceutical composition, comprising: a therapeutically effective amount of a salt of flecainide, wherein the pharmaceutical composition is in the form of a liquid solution that has the salt of flecainide at a concentration above 60 mg/mL. In some embodiments, the pharmaceutical composition further comprises a cyclodextrin. In some embodiments, the presence of cyclodextrin increases the solubility of the salt of flecainide as compared to a corresponding formulation without the cyclodextrin. In some embodiments, the solution has pH above 5.5 when the pH is measured at room temperature. In some embodiments, the pharmaceutical composition further comprises a cyclodextrin, and the pH of the solution is above 5.5 when the pH is measured at room temperature. In some embodiments, the presence of cyclodextrin renders it possible to increase pH of the solution without compromising the solubility of the salt of flecainide, as compared to a corresponding solution without the cyclodextrin.

In some embodiments, the pharmaceutical composition or formulation provided herein enables delivery of more pharmaceutically active ingredient, e.g., flecainide, to the subject. In some embodiments, the subject pharmaceutical composition or formulation has an increased flecainide concentration as compared to a corresponding flecainide formulation (e.g., flecainide acetate water solution which has a solubility around 60 mg/mL). In some cases, the increased flecainide concentration increases the delivery speed when the composition is nebulized and administered via inhalation. In some embodiments, the increased flecainide concentration shortens the inhalation duration as a given dose can be delivered at a higher speed as compared to a corresponding formulation with a lower concentration of flecainide. Shorter inhalation duration can improve subject compliance, which can further increase the delivery efficiency of the drug.

In some embodiments, the pharmaceutical composition or formulation provided herein reduces adverse cough of the subject while inhaling, has improved organoleptic properties, and improves overall patient experience of inhalation. In some embodiments, the improved overall inhalation experience results in better compliance with the full inhalation program. In some embodiments, more effective drug delivery is achieved when the subject has better inhalation compliance, and thus more drug is delivered.

In some embodiments, the subject pharmaceutical composition or formulation has improved pharmacokinetics or pharmacodynamics parameter(s), for instance, the peak plasma level of flecainide (Cₘₐₓ) can be higher with the subject pharmaceutical composition as compared to a corresponding pharmaceutical composition in which the concentration of the salt of flecainide is lower. In some embodiments, the Cₘₐₓ achieved with the subject pharmaceutical composition is at least 10%, 20%, 30%, 40%, 50%, 60%, 70%, 80%, 90%, or 100%, or 1.1, 1.2, 1.3, 1.4, 1.5, 1.6, 1.7, 1.8, 1.9, 2.0 or greater fold higher as compared to a corresponding pharmaceutical composition in which the concentration of the salt of flecainide is lower.

In some embodiments, a pharmaceutical composition with a low concentration of acid or a high pH, e.g., at most about 10 mM acetic acid, e.g., about 5 mM acetic acid, or e.g. a pH of higher than 5.5, e.g., a pH of 5.9 when the pH is measured at room temperature, has a higher Cₘₐₓ as compared to a corresponding pharmaceutical composition in which the concentration of the acid is higher or the pH is lower, e.g., at least about 50 mM acetic acid, e.g., about 90 mM acetic acid, or e.g., a pH of at most 5.5, e.g., a pH of about 5.2 when the pH is measured at room temperature. In some embodiments, the Cₘₐₓ achieved with the subject pharmaceutical composition that has a low concentration of acid or a high pH is at least 10%, 20%, 30%, 40%, 50%, 60%, 70%, 80%, 90%, or 100%, or 1.1, 1.2, 1.3, 1.4, 1.5, 1.6, 1.7, 1.8, 1.9, 2.0 or greater fold higher as compared to a corresponding pharmaceutical composition in which the concentration of the acid is higher or the pH is lower. In some embodiments, the Cₘₐₓ achieved with the subject pharmaceutical composition that has at most about 10 mM acetic acid, e.g., about 5 mM acetic acid, or a pH of higher than 5.5, e.g., a pH of 5.9 when the pH is measured at room temperature is at least 10%, 20%, 30%, 40%, 50%, 60%, 70%, 80%, 90%, or 100%, or 1.1, 1.2, 1.3, 1.4, 1.5, 1.6, 1.7, 1.8, 1.9, 2.0 or greater fold higher as compared to a corresponding pharmaceutical composition in which the concentration of the acetic acid is about 90 mM, or the pH is about 5.2.

In one aspect of the present disclosure, provided herein is a unit dose of a pharmaceutical composition provided herein. In some embodiments, the unit dose comprises about 50 mg to about 350 mg of the salt of flecainide. In another aspect, provided herein are kits comprising the pharmaceutical composition or the unit dose provided herein and instructions for use of the pharmaceutical composition for treatment of a heart condition (e.g., cardiac arrhythmia, e.g., atrial arrhythmia).

In one aspect of the present disclosure, provided is a system comprising a pharmaceutical composition provided herein and a nebulizer. In some embodiments, the system further comprises instructions for use of the nebulizer and the pharmaceutical composition for treatment of a heart condition. In some embodiments, the system comprises: a pharmaceutical composition that comprises a salt of flecainide, a cyclodextrin, and an acid; a nebulizer configured to inhalationally administer the pharmaceutical composition as droplets having a mass median aerodynamic diameter of less than 10 μm; and instructions for use of the nebulizer to inhalationally administer the pharmaceutical composition in an aerosolized dose that contains from about 50 mg to about 150 mg of the salt of flecainide, wherein the pharmaceutical composition is in the form of a liquid solution that has (i) the salt of flecainide at a concentration of from about 65 mg/mL to about 95 mg/mL, (ii) the cyclodextrin at a concentration of from about 10% (w/v) to about 30% (w/v) of the solution; and (iii) a pH of from about 5.5 to about 6.5 when the pH is measured at room temperature.

### Cyclodextrins

In some aspects of the present disclosure, a cyclodextrin is used as a solubility enhancer of a salt of flecainide. Cyclodextrins are cyclic carbohydrates derived from starch. The unmodified cyclodextrins differ by the number of glucopyranose units joined together in the cylindrical structure. The parent cyclodextrins contain 6, 7, or 8 glucopyranose units and are referred to as α-, β-, and γ-cyclodextrin respectively. Each cyclodextrin subunit can have secondary hydroxyl groups at the 2 and 3 positions and a primary hydroxyl group at the 6-position. The cyclodextrins can be pictured as hollow truncated cones with hydrophilic exterior surfaces and hydrophobic interior cavities. In aqueous solutions, these hydrophobic cavities can provide a haven for hydrophobic organic compounds that can fit all or part of their structure into these cavities. This process, known as inclusion complexation, can result in increased apparent aqueous solubility and stability for the complexed drug.

The cyclodextrin in a pharmaceutical composition provided herein can include, but not limited to, α-cyclodextrin, β-cyclodextrin, γ-cyclodextrin, derivatized α-cyclodextrins, derivatized β-cyclodextrins, and derivatized γ-cyclodextrins. Non-limiting examples of cyclodextrin that can be used in the subject pharmaceutical composition include α-cyclodextrin, β-cyclodextrin, γ-cyclodextrin, hydroxypropyl-β-cyclodextrin, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin dimaltosyl-β-cyclodextrin, succinyl-β-cyclodextrin, 6A-amino-6A-deoxy-N-(3-hydroxypropyl)-β-cyclodextrin, sulfobutylether-β-cyclodextrin, sulfobutylether-γ-cyclodextrin, sulfoalkylether-β-cyclodextrins, and sulfoalkylether-γ-cyclodextrins. In some embodiments, the pharmaceutical composition comprises hydroxypropyl-β-cyclodextrin (HPβCD). In some embodiments, the pharmaceutical composition comprises more than one species of cyclodextrins, such as, 2, 3, 4, 5, 6, 7, 8, 9, 10 or more different species of cyclodextrins. In some embodiments, the pharmaceutical composition comprises HPβCD and one or more other cyclodextrins, such as, 1, 2, 3, 4, 5, 6, 7, 8, 9, 10 or more other different species of cyclodextrins.

The subject pharmaceutical composition can comprise a cyclodextrin at a concentration of at least about 1% (w/v) of the solution, such as at least about 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 28%, 30%, 35%, 40%, 45%, 50%, 55%, 60%, 65%, 70%, 75%, 80%, 85%, 90%, 100%, or more (w/v) of the solution. In some embodiments, the pharmaceutical composition comprises a cyclodextrin at a concentration of from about 1% (w/v) to about 80% (w/v) of the solution, such as from about 2% (w/v) to about 70% (w/v), from about 2% (w/v) to about 60% (w/v), from about 2% (w/v) to about 50% (w/v), from about 2% (w/v) to about 40% (w/v), from about 2% (w/v) to about 30% (w/v), from about 2% (w/v) to about 20% (w/v), from about 2% (w/v) to about 15% (w/v), from about 2% (w/v) to about 10% (w/v), from about 2% (w/v) to about 8% (w/v), from about 2% (w/v) to about 5% (w/v), from about 5% (w/v) to about 80% (w/v), from about 5% (w/v) to about 70% (w/v), from about 5% (w/v) to about 60% (w/v), from about 5% (w/v) to about 50% (w/v), from about 5% (w/v) to about 40% (w/v), from about 5% (w/v) to about 30% (w/v), from about 5% (w/v) to about 20% (w/v), from about 5% (w/v) to about 15% (w/v), from about 5% (w/v) to about 12% (w/v), from about 5% (w/v) to about 10% (w/v), from about 10% (w/v) to about 60% (w/v), from about 10% (w/v) to about 50% (w/v), from about 10% (w/v) to about 40% (w/v), from about 10% (w/v) to about 30% (w/v), from about 20% (w/v) to about 30% (w/v), from about 10% (w/v) to about 25% (w/v), from about 19% (w/v) to about 25% (w/v), from about 19.5% (w/v) to about 25% (w/v), from about 20% (w/v) to about 25% (w/v), from about 20.5% (w/v) to about 25% (w/v), from about 21% (w/v) to about 25% (w/v), from about 21.5% (w/v) to about 25% (w/v), from about 22% (w/v) to about 25% (w/v), from about 22.5% (w/v) to about 25% (w/v), from about 23% (w/v) to about 25% (w/v), from about 10% (w/v) to about 20% (w/v), or from about 10% (w/v) to about 15% (w/v) of the solution. In some embodiments, the pharmaceutical composition comprises a cyclodextrin at a concentration of about 2%, 3%, 4%, 5%, 6%, 7%, 8%, 9%, 10%, 11%, 12%, 13%, 14%, 15%, 16%, 17%, 18%, 19%, 20%, 20.5%, 21%, 21.5%, 22%, 22.5%, 23%, 23.5%, 24%, 24.5%, 25%, 25.5%, 26%, 26.5%, 27%, 27.5%, 28%, 28.5%, 29%, 29.5%, 30%, 31%, 32%, 33%, 34%, 35%, 38%, 40%, 45%, 50%, 55%, 60%, 65%, 70%, 75%, or 80% (w/v) of the solution.

In some embodiments, the concentration of the cyclodextrin contributes to the viscosity of the solution, which can reduce the nebulization efficiency (or rate) of the solution. For instance, in some cases, the higher the concentration of the cyclodextrin is, the higher viscosity of the solution is. In some cases, the concentration of the cyclodextrin in the pharmaceutical composition is controlled so that the viscosity of the solution is not higher than a reference value, such as about 3.1 cP, 3.2 cP, 3.3 cP, 3.4 cP, 3.5 cP, 3.6 cP, 3.7 cP, 3.8 cP, 3.9 cP, 4.0 cP, 4.1 cP, 4.2 cP, 4.3 cP, 4.4 cP, 4.5 cP, 4.6 cP, 4.7 cP, 4.8 cP, 4.9 cP, or 5.0 cP. In some cases, the concentration of the cyclodextrin in the pharmaceutical composition is at most about 2%, 5%, 8%, 10%, 12%, 15%, 18%, 20%, 20.5%, 21%, 21.5%, 22%, 22.5%, 23%, 23.5%, 24%, 24.5%, 25%, 25.5%, 26%, 26.5%, 27%, 27.5%, 28%, 28.5%, 29%, 29.5%, 30%, 31%, 32%, 33%, 34%, 35%, 38%, 40%, 45%, 50%, 55%, 60%, 65%, 70%, 75%, or 80% (w/v) of the solution.

In some embodiments, the pharmaceutical composition comprises HPβCD at a concentration of about 20% (w/v) of the solution. In some embodiments, the pharmaceutical composition comprises HPβCD at a concentration of about 22.5% (w/v) of the solution. In some embodiments, the pharmaceutical composition comprises HPβCD at a concentration of about 20% (w/v) of the solution.

### Acids

Some aspects of the present disclosure relate to use of one or more acids in the pharmaceutical composition. In some cases, the acid enhances solubility of flecainide. In some cases, flecainide freebase has a low solubility, e.g., in water. In some cases, certain salts of flecainide have higher solubility as compared to other salts of flecainide and flecainide freebase. For instance, flecainide acetate can have a higher solubility as compared to some other flecainide salts, as demonstrated in Example 1. In some cases, acid is provided in the pharmaceutical composition to provide anion for flecainide salt formation and sufficiently low pH to ensure the solubility of the flecainide salt.

In some cases, a mixture of more than one acid can increase flecainide solubility as compared to a single acid. In some instances, the pharmaceutical composition comprises acetic acid as the single acid. In some instances, the pharmaceutical composition comprises citric acid as the single acid. In some cases, the pharmaceutical composition comprises a mixture of different acids. In some cases, the pharmaceutical composition comprises lactic acid. In some cases, the pharmaceutical composition comprises L-(+)-lactic acid. In some cases, the pharmaceutical composition comprises D-(−)-lactic acid. In some cases, the pharmaceutical composition comprises a mixture of D-(−)-lactic acid and L-(+)-lactic acid, i.e. the pharmaceutical composition comprises DL-lactic acid. In some cases, there are equal amounts of D-(−)-lactic acid and L-(+)-lactic acid in the pharmaceutical composition. In some cases, the pharmaceutical composition comprises ascorbic acid. Non-limiting examples of the acids that can be used in the subject pharmaceutical compositions and methods of treatment include any suitable organic or inorganic acid, such as any GRAS (Generally Recognized As Safe) listed acid, e.g., acetic acid, aconitic acid, adipic acid, alginic acid, benzoic acid, caprylic acid, citric acid, cholic acid, formic acid, lactic acid (e.g., D-(−)-lactic acid or L-(+)-lactic acid), linoleic acid, malic acid, maleic acid, propionic acid, stearic acid, succinic acid, sulfuric acid, tannic acid, tartaric acid, glutamic acid, hydrochloric acid, phosphoric acid, ascorbic acid, erythorbic acid, sorbic acid, or thiodipropionic acid, or any other acid that is not listed in GRAS but is pharmaceutically acceptable in the subject pharmaceutical composition.

In some cases, the pharmaceutical composition comprises a mixture of different acids, such as 2, 3, 4, 5, 6, 7, 8, 9, 10 or more different acids. In some cases, the pharmaceutical composition comprises one of the following: acetic acid and nitric acid; acetic acid and sulfuric acid; acetic acid and citric acid; acetic acid, nitric acid, and sulfuric acid; acetic acid, nitric acid, and citric acid; acetic acid, citric acid, and sulfuric acid; or acetic acid, nitric acid, citric acid, and sulfuric acid.

In some embodiments, the pharmaceutical composition has a pH that is above 5.5 when the pH is measured at room temperature, such as above 5.6, above 5.7, above 5.8, above 5.9, above 6.0, above 6.1, above 6.2, above 6.3, above 6.4, above 6.5, above 6.6, above 6.7, or above 6.8 when the pH is measured at room temperature. In some cases, the pharmaceutical composition is acidic at room temperature, e.g., having a pH at most 6.9, at most 6.8, at most 6.7, at most 6.6, at most 6.5, at most 6.4, at most 6.3, at most 6.2, at most 6.1, at most 6.0, at most 5.9, at most 5.8, at most 5.7, or at most 5.6 when the pH is measured at room temperature. In some cases, the pharmaceutical composition has a pH that is from about 5.5 and about 6.5 when the pH is measured at room temperature, such as from about 5.6 and about 6.4, from about 5.7 and about 6.3, from about 5.8 and about 6.2, or from about 5.9 and about 6.1 when the pH is measured at room temperature. In some instances, the pharmaceutical composition has a pH of about 5.5, 5.6, 5.7, 5.8, 5.9, 6.0, 6.1, 6.2, 6.3, or 6.4 when the pH is measured at room temperature. In some examples, the pharmaceutical composition has a pH of about 5.5 when the pH is measured at room temperature. In some embodiments, the pH of the pharmaceutical composition is titrated by a pH buffer as described herein.

In some embodiments, the concentration of the acid in the pharmaceutical composition is about 2 mM to about 200 mM, such as about 2 mM to about 180 mM, from about 2 mM to about 150 mM, from about 2 mM to about 120 mM, from about 2 mM to about 100 mM, from about 2 mM to about 80 mM, from about 2 mM to about 60 mM, from about 2 mM to about 50 mM, from about 2 mM to about 40 mM, from about 2 mM to about 30 mM, from about 2 mM to about 20 mM, from about 2 mM to about 10 mM, from about 2 mM to about 8 mM, 2 mM to about 6 mM, from about 5 mM to about 200 mM, from about 5 mM to about 150 mM, from about 5 mM to about 120 mM, from about 5 mM to about 100 mM, from about 5 mM to about 80 mM, from about 5 mM to about 60 mM, from about 5 mM to about 50 mM, from about 5 mM to about 40 mM, from about 5 mM to about 30 mM, from about 5 mM to about 20 mM, from about 5 mM to about 10 mM, from about 5 mM to about 8 mM, from about 10 mM to about 200 mM, from about 10 mM to about 150 mM, from about 10 mM to about 120 mM, from about 10 mM to about 100 mM, from about 10 mM to about 90 mM, from about 10 mM to about 80 mM, from about 10 mM to about 70 mM, from about 10 mM to about 60 mM, from about 10 mM to about 50 mM, from about 10 mM to about 40 mM, from about 10 mM to about 30 mM, from about 10 mM to about 20 mM, from about 20 mM to about 200 mM, from about 20 mM to about 150 mM, from about 20 mM to about 100 mM, from about 20 mM to about 90 mM, from about 20 mM to about 80 mM, from about 20 mM to about 70 mM, from about 20 mM to about 60 mM, from about 20 mM to about 50 mM, from about 20 mM to about 40 mM, from about 20 mM to about 30 mM, from about 40 mM to about 200 mM, from about 40 mM to about 150 mM, from about 40 mM to about 120 mM, from about 40 mM to about 100 mM, from about 40 mM to about 90 mM, from about 40 mM to about 80 mM, from about 40 mM to about 70 mM, from about 40 mM to about 60 mM, or about 40 mM to about 50 mM. In some embodiments, the concentration of the acid in the pharmaceutical composition is at most about 200 mM, such as at most about 180 mM, at most about 160 mM, at most about 150 mM, at most about 140 mM, at most about 120 mM, at most about 100 mM, at most about 90 mM, at most about 80 mM, at most about 70 mM, at most about 60 mM, at most about 50 mM, at most about 40 mM, at most about 30 mM, at most about 20 mM, at most about 10 mM, at most about 9 mM, at most about 8 mM, at most about 7 mM, at most about 6 mM, at most about 5 mM, at most about 4 mM, at most about 3 mM, at most about 2 mM, or at most about 1 mM. In some embodiments, the concentration of the acid in the pharmaceutical composition is about 100 mM, about 90 mM, about 80 mM, about 70 mM, about 60 mM, about 50 mM, about 40 mM, about 30 mM, about 20 mM, about 10 mM, about 9 mM, about 8 mM, about 7 mM, about 6 mM, about 5 mM, about 4 mM, about 3 mM, about 2 mM, or about 1 mM. In some embodiments, the concentration of the acid in the pharmaceutical composition is about 20 mM. In some embodiments, the concentration of the acid in the pharmaceutical composition is about 5 mM.

Without wishing to be bound to a particular theory, the concentration of the acid in the pharmaceutical composition can contribute to the pharmaceutical application of the composition. In some embodiments, the acid concentration and thus the pH of the solution can influence solubility of the flecainide salt in the formulation, therefore affecting the concentration of the flecainide salt. As discussed above, the concentration of the flecainide salt can contribute to the delivery efficiency and rate of the pharmaceutical composition. In some embodiments, the acid concentration can influence the organoleptic properties of the pharmaceutical composition. For instance, the lower the pH of the solution is, the more irritating the solution can be to the mouth, the nose, the pharynx, or other parts of the respiratory system, particularly the upper respiratory system. Irritation of the solution can induce cough or other adverse reactions, or reduced compliance of the subject, therefore adversely affecting the delivery efficiency of the pharmaceutical composition.

In some embodiments, the pharmaceutical composition provided herein does not induce coughing reflex when being inhaled by a subject. In some embodiments, the pharmaceutical composition provided herein induces coughing reflex less frequently when being inhaled by a subject as compared to a corresponding pharmaceutical composition that has a higher acid concentration or lower pH, such as about 10%, 20%, 30%, 50%, 60%, 70%, 80%, 90%, or 100%, or 1.1, 1.2, 1.3, 1.4, 1.5, 1.6, 1.7, 1.8, 1.9, 2.0 or greater fold lower as compared to the corresponding pharmaceutical composition. In some embodiments, a subject receiving inhalation administration of the pharmaceutical composition reports less severe discomfort when inhaling the composition, or has lower incidence of reporting discomfort when inhaling the composition, as compared to a corresponding pharmaceutical composition that has a higher acid concentration or lower pH, such as about 10%, 20%, 30%, 50%, 60%, 70%, 80%, 90%, or 100%, or 1.1, 1.2, 1.3, 1.4, 1.5, 1.6, 1.7, 1.8, 1.9, 2.0 or greater fold lower as compared to the corresponding pharmaceutical composition.

Without wishing to be bound by a certain theory, the presence of a cyclodextrin in the pharmaceutical composition can reduce the concentration of acid that is required to achieve a desirable concentration of flecainide salt according to some aspects of the present disclosure. In some embodiments, the presence of a cyclodextrin (e.g., HPβCD) increases flecainide solubility, e.g., through inclusion complexation that “dissolves” flecainide salt (e.g., flecainide acetate) inside the cavity of the cyclodextrin. The inclusion complexation, on the other hand, can reduce the reliability of flecainide salt on the acid (or the low pH) to be dissolved in the solution. Therefore, as a result, the introduction of cyclodextrin (e.g., HPβCD) can lead to reduction of acid concentration in the solution, both of which synergistically lead to increased flecainide concentration in the solution, improved organoleptic properties, increased delivery speed, and improved delivery efficiency.

### Sweeteners and Organoleptic Properties

In some embodiments, the pharmaceutical composition provided herein comprises a sweetener to improve the organoleptic properties of the composition. The sweetener can be a natural sweet substance, e.g. certain sugars, or an artificial sweetener. Without wishing to be bound to a certain theory, the presence of the sweetener in the pharmaceutical composition can improve the organoleptic properties of the composition. In some cases, the presence of the sweetener in the pharmaceutical composition can improve the compliance of the subject. presence of the sweetener in the pharmaceutical composition can increase the delivery efficiency of the composition. In some embodiments, the presence of the sweetener in the pharmaceutical composition can enhance the therapeutic effects of the composition.

Non-limiting examples of artificial sweeteners that can be used in the pharmaceutical composition include acesulfame potassium, aspartame, cyclamate, mogrosides, saccharin, stevia, sucralose, neotame, and sugar alcohols (e.g., erythritol, hydrogenated starch hydrolysates, isomalt, lactitol, maltitol, mannitol, sorbitol, and xylitol), such as those used in commercial products, like Sweet n′ low powder sweetener, Truvia powder sweetener, Equal (aspartame), Stevia powder sachet, Aspen Naturals liquid stevia, Now Better Stevia liquid sweetener, Sweet N′ Low liquid sweetener, Quick Sweet: Neotame liquid sweetener, or Splenda powder sachet, or pharmaceutically acceptable salts thereof In some embodiments, the pharmaceutical composition comprises saccharin. In some embodiments, the pharmaceutical composition comprises a salt of saccharin. In some embodiments, the pharmaceutical composition comprises saccharin sodium.

Natural sweet substances that can be used in the pharmaceutical composition include, but not limited to, sucrose, agave, brown sugar, confectioner's (powdered) sugar, corn syrup, dextrose, fructose, fruit juice concentrate, glucose, high-fructose corn syrup, honey, invert sugar, lactose, malt sugar, maltose, maple syrup, molasses, nectars, raw sugar, and syrup. Sugars can increase the viscosity of the liquid solution, thus the concentration of any sugar added into the pharmaceutical composition, in some embodiments, is tightly controlled below a certain threshold value.

In some embodiments, the concentration of the sweetener, e.g., artificial sweetener, in the pharmaceutical composition is from about 0.001% (w/v) to about 1% (w/v) of the solution, such as 0.002% to 1%, 0.005% to 1%, 0.01% to 1%, 0.02% to 1%, 0.05% to 1%, 0.08% to 1%, 0.1% to 1%, 0.2% to 1%, 0.5% to 1%, 0.8 to 1%, 0.002% to 0.5%, 0.005% to 0.5%, 0.01% to 0.5%, 0.02% to 0.5%, 0.05% to 0.5%, 0.08% to 0.5%, 0.1% to 0.5%, 0.2% to 0.5%, 0.005% to 0.1%, 0.01% to 0.1%, 0.02% to 0.1%, 0.05% to 0.1%, 0.08% to 0.1%, 0.005% to 0.05%, 0.01% to 0.05%, or 0.02% to 0.05% (w/v) of the solution. In some embodiments, the concentration of the sweetener, e.g., artificial sweetener, in the pharmaceutical composition is at least about 0.001%, at least about 0.002%, at least about 0.005%, at least about 0.01%, at least about 0.02%, at least about 0.05%, at least about 0.08%, at least about 0.1%, at least about 0.2%, at least about 0.5%, or at least about 0.8, or at least about 1% (w/v) of the solution. In some embodiments, the concentration of the sweetener, e.g., artificial sweetener, in the pharmaceutical composition is at most about 0.001%, at most about 0.002%, at most about 0.005%, at most about 0.01%, at most about 0.02%, at most about 0.05%, at most about 0.08%, at most about 0.1%, at most about 0.2%, at most about 0.5%, or at most about 0.8, or at most about 1% (w/v) of the solution.

### Preparation

In some aspects, the present disclosure provides a method of preparing a liquid pharmaceutical composition that comprises an antiarrhythmic pharmaceutical agent. In some cases, the method comprises combining: (a) water; (b) a pH adjusting agent; (c) flecainide or a pharmaceutically acceptable salt thereof; and (d) a cyclodextrin. In some cases, the water used in preparing the formulation is sterilized. In some cases, the water used in preparing the formulation is water for injection. In some cases, all the starting materials are sterilized by established technologies that meet the standards for medical use.

In some cases, the method of preparation includes (a) providing the water; (b) contacting the portion of water with the flecainide or pharmaceutically acceptable salt thereof, the cyclodextrin, and the pH adjusting agent in a vessel; and (c) adding a subsequent portion of the water to the vessel to provide the pharmaceutical composition.

In some cases, in the composition prepared by the method provided herein, a concentration of the flecainide or a pharmaceutically acceptable salt thereof is from about 65 mg/mL to about 95 mg/mL in the pharmaceutical composition, a concentration of the cyclodextrin in the pharmaceutical composition is from about 10% (w/v) to about 30% (w/v); and a room-temperature pH in the pharmaceutical composition of from about 5.5 to about 6.5.

In some cases, the pH adjusting agent comprises an ion selected from the group consisting of: acetate, citrate, nitrate, chloride, sulfate, maleate, tartrate, phosphate, aconitate, adipate, ascorbate, benzoate, caprylate, cholate, formate, glutamate, lactate, propionate, sorbate, stearate, and succinate. In some cases, the pH adjusting agent comprises a pH buffer. In some cases, the pH adjusting agent comprises an acid or a base. In some cases, the pH adjusting agent comprises an acid. In some cases, the pH adjusting agent is selected from the group consisting of: acetic acid, citric acid, nitric acid, hydrochloric acid, sulfuric acid, maleic acid, tartaric acid, phosphoric acid, aconitic acid, adipic acid, ascorbic acid, benzoic acid, caprylic acid, cholic acid, formic acid, glutamic acid, lactic acid, propionic acid, sorbic acid, stearic acid, and succinic acid. In some cases, the pH adjusting agent is selected from the group consisting of: acetic acid, citric acid, nitric ac-id, hydrochloric acid, and sulfuric acid. In some cases, the pH adjusting agent comprises a mixture of acids, including, but not limited to, acetic acid, citric acid, nitric acid, hydrochloric acid, and sulfuric acid. In some cases, the pH adjusting agent comprises acetic acid. In some cases, the pH adjusting agent comprises citric acid.

In some cases, the pH adjusting agent is added to a concentration at about 2 mM to about 50 mM. In some cases, the pH adjusting agent is added to a concentration at about 2 mM to about 10 mM. In some cases, the pH adjusting agent comprises acetic acid. In some cases, the concentration in the pharmaceutical composition of the acetic acid is about 5 mM. In some cases, the pH adjusting agent comprises citric acid. In some cases, the concentration in the pharmaceutical composition of the citric acid is about 5 mM.

In some cases, the method of preparation includes adding cyclodextrin to the solution. In some cases, the cyclodextrin to be added comprises α-cyclodextrin, β-cyclodextrin, γ-cyclodextrin, derivatized α-cyclodextrins, derivatized β-cyclodextrins, or derivatized γ-cyclodextrins. In some cases, the cyclodextrin comprises α-cyclodextrin, β-cyclodextrin, γ-cyclodextrin, hydroxypropyl-β-cyclodextrin, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin dimaltosyl-β-cyclodextrin, succinyl-β-cyclodextrin, 6A-amino-6A-deoxy-N-(3-hydroxypropyl)-β-cyclodextrin, sulfobutyl-ether-β-cyclodextrin, sulfobutylether-γ-cyclodextrin, sulfoalkylether-β-cyclodextrins, or sulfoalkylether-γ-cyclodextrins. In some cases, the cyclodextrin comprises hydroxypropyl-β-cyclodextrin. In some cases, the concentration of the cyclodextrin in the pharmaceutical com-position is from about 10% (w/v) to about 30% (w/v).

In some cases, the method of preparation includes adding a sweetener to the solution. In some cases, the sweetener is selected from the group consisting of: acesulfame potassium, aspartame, cyclamate, mogrosides, saccharin, stevia, sucralose, neotame, mannitol, sorbitol, xylitol, lactitol, isomalt, maltitol, and pharmaceutically acceptable salts thereof. In some cases, the sweetener comprises saccharin. In some cases, the sweetener comprises a salt of saccharin. In some cases, the sweetener comprises saccharin sodium. In some cases, the sweetener is added to a concentration at from about 0.001% (w/v) to about 1% (w/v). In some cases, the sweetener is added to a concentration at from about 0.001% (w/v) to about 0.05% (w/v). In some cases, the sweetener is added to a concentration at from about 0.001% (w/v) to about 0.01% (w/v).

In some cases, the method of preparation includes adding a pharmaceutically acceptable salt of flecainide, such as, flecainide acetate, flecainide hydrochloride, flecainide citrate, flecainide phosphate, or flecainide nitrate. In some cases, the pharmaceutically acceptable the salt of flecainide comprises flecainide acetate. In some cases, the pharmaceutically acceptable the salt of flecainide comprises flecainide hydrochloride.

In some cases, the method of preparation further includes packaging the pharmaceutical composition in unit dose form. For instance, the unit dose form can include about 50 mg to about 350 mg of the pharmaceutically acceptable salt of flecainide. For instance, the unit dose form comprises about 60 mg to about 150 mg of the pharmaceutically acceptable salt of flecainide, such as about 75 mg to about 125 mg, about 250 mg to about 350 mg, or about 150 mg to about 250 mg, such as about 90 mg, about 120 mg, or about 200 mg of the pharmaceutically acceptable salt of flecainide.

### EXAMPLES

The following examples are provided to further illustrate some embodiments of the present disclosure, but are not intended to limit the scope of the disclosure; it will be understood by their exemplary nature that other procedures, methodologies, or techniques known to those skilled in the art may alternatively be used.

### Example 1: Analysis of Anthropometric Predictors for Successful Cardioversion of Recent-Onset Atrial Fibrillation to Sinus Rhythm with Orally Inhaled Flecainide

A flecainide acetate oral inhalation solution for acute cardioversion of recent-onset symptomatic AF was administered to 80 patients to determine if height, weight, and body mass index (BMI) are predictors of successful cardioversion of AF to sinus rhythm (SR) with the inhalation solution.

### Methods

The flecainide formulation used in this study is described in TABLE 1.

TABLE 1
Formulation in clinical study

| Inhalation solution | Formulation | |
|---|---|---|
| [concentration (mg/mL)] | Formulation A | |
| Flecainide Dose (eTLD) | 120 | mg |
| Concentration of | 75 | |
| flecainide acetate (mg/mL) | | |
| Other components: | | |
| Acetic acid buffer | 5 | mM |
| Sodium saccharin | 0.75 | mM |

| Hydroxypropyl β- | 20% |
|---|---|
| cyclodextrin (w/v) | |
| pH* | 5.9 |
| Estimated Duration of | 8 |
| Inhalation Regimen (min) | |
| Dosing pattern | 3.5 minutes inhalation, followed |
| | by 1 minute break, followed by |
| | 3.5 minutes inhalation |
| Approximate Rate of Dose | 15.0 |
| Delivery (mg/min) | |
| Inhalation Regimen | Dose/inhale to conversion^{#} |

^{#}‘Dose/inhale to conversion’ = Inhale until the time of conversion of AF to SR, or continue to inhale until the full dose is administered, whichever occurs first
*NaOH is added as necessary to achieve specified pH

In this study, for inhalation delivery of flecainide, an estimated total lung dose (eTLD) of 120 mg were calculated to account for losses of flecainide in the inhalation device and losses of flecainide in subjects' mouth and throat. An 120 mg eTLD was thereby used to denote the dose that actually reached the lungs of the subjects. By design, in all nebulizers there can be a residual volume or mass of drug solution that stays in the nebulizer, and there can also be a percentage of the aerosol caught by subject's throat and mouth. For instance, in this study, it was estimated that 30% of the aerosol was lost in subject's throat and mouth. Therefore the eTLD would be: eTLD=(100-30) %*amount of aerosolized drug that left nebulizer=70%*(amount of drug placed in nebulizer−amount of drug staying in nebulizer).

Patient baseline characteristics are summarized in TABLE 2.

TABLE 2

| | | | | | Obese/Severe | |
|---|---|---|---|---|---|---|
| | Normal | | Overweight | | Obese | |
| Characteristic | (N = 33)* | | (N = 33)* | | (N = 17)* | |
| Age (y) | 59.8 ± 10.3 | | 60.7 ± 12.7 | | 56.9 ± 15.6 | |
| Male sex, n (%) | 24 | (72.7%) | 22 | (66.7%) | 10 | (58.8%) |
| White, n (%) | 32 | (97.0%) | 30 | (90.9%) | 16 | (94.1%) |
| Body mass index (kg/m2) | 23.4 ± 1.6 | | 27.3 ± 1.4 | | 32.6 ± 2.3 | |
| Hypertension, n (%) | 7 | (21.2%) | 9 | (27.3%) | 8 | (47.1%) |
| Hyperlipidemia, n (%) | 7 | (21.2%) | 11 | (33.3%) | 4 | (23.5%) |
| Diabetes, n (%) | 0 | (0.0%) | 0 | (0.0%) | 1 | (5.9%) |
| NYHA HF Class I, n (%) | 4 | (12.1%) | 2 | (6.1%) | 2 | (11.8%) |
| NYHA HF Class II, n (%) | 0 | (0%) | 1 | (3.0%) | 0 | (0%) |
| CHA₂DS₂-VASc Score | 0.9 ± 1.0 | | 1.4 ± 1.6 | | 1.5 ± 1.2 | |
| AF symptom duration, | 15.2 ± 12.4 | | 11.0 ± 6.7 | | 11.9 ± 11.1 | |
| hours | | | | | | |
| First AF Episode, n (%) | 15 | (45.5%) | 11 | (33.3%) | 10 | (58.8%) |
| Recurrent Paroxysmal | 17 | (51.5%) | 20 | (60.6%) | 6 | (35.3%) |
| AF Episode, n (%) | | | | | | |
| AF Post-Cardiac | 1 | (3.0%) | 2 | (6.1%) | 1 | (5.9%) |
| Ablation, n (%) | | | | | | |

| | | | |
|---|---|---|---|
| # Previous AF Episodes | 1.3 ± 0.6 | 2.9 ± 3.9 | 2.4 ± 1.6 |
| (excludes 1^{st} episode | | | |

*Data are mean ± SD unless otherwise noted. Safety population (N = 83).

### Nebulizer Configuration.

The AeroEclipse®II BAN inhaler was used for nebulization and inhalation of the exemplary flecainide formulations. It is available as an approved device in several countries across the world including European countries, USA, and Canada. The inhaler is a hand-held, breath actuated, jet nebulizer which operates through a source of compressed air available as medical air in the hospital ER. The AeroEclipse®BAN delivers a high respirable dose and an optimal particle size to reach the deeper lung regions to enable faster drug absorption. Flecainide acetate inhalation solution was transferred from the vial into the reservoir of the AeroEclipse®II BAN at a volume corresponding to the required dose to the lung. To administer an estimated total lung dose of 120 ng flecainide acetate, the nebulizer was filled with 4.2 mL of Formulation A described in TABLE 1. The total volume filled in each dosing cup was based on the assumption of a 70% (v/v) lung deposition, a 12.5% (v/v) loss due to fugitive aerosol based on in-vitro experiments, and device retention of approximately 1.5 mL of the solution.

### Inhalation Guidelines.

Participants in the study were administered the study medications according to the following guidelines:
Subject self-administers inhalation using the AeroEclipse® II BAN
Subject is seated upright in a comfortable chair or adjustable bed with table that has adjustable height in front (e.g. overbed table on casters) where the subject can rest his/her arms (e.g. overbed table on casters) from approximately 30 minutes prior to the start of inhalation up to at least 15 minutes after the last inhalation is completed. The setting at the site allows for subject to remain in this upright sitting position while linked to cardiac monitoring systems, while blood samples are being collected and while self-administering the inhalation solution which is linked to compressed air.
The subject receives clear instructions from trained study personnel on how to self-administer the treatment in accordance with the instructions for use.
A topical oral anesthetic spray (e.g., containing lidocaine [e.g., Medica] or phenol [e.g., Chloraseptic]) or lozenge [e.g., Trachitol or Cepacol] may be applied to the back of the subject's throat prophylactically if not contraindicated, to improve the tolerability of the inhalation procedure.
A sugar-containing spray or lozenge (e.g., for dry mouth) may be applied or used prophylactically if not contraindicated, to improve the tolerability of the inhalation procedure.
The subject may practice the inhalation procedure (without flecainide and for approximately 1 minute, for example) prior to study drug administration.

Once the inhalation pattern is established, the subject should not remove inhaler device from the mouth for the required inhalation time. If the subject removes the nebulizer for any reason (e.g., cough, excessive saliva, etc.) for >30 seconds, then the inhalation time for that inhalation should be extended by the length of the interruption. Subjects can elect to terminate inhalation of study medication at any time, for any reason.

### Results

Logistic regression was performed to identify predictors of cardioversion success at 90 minutes post-dose, and potential interactions were examined by linear regression. Data are presented for all patients receiving 120 mg flecainide acetate who did not have flecainide present in their pre-dosing blood samples. Analyzed factors are summarized in TABLE 3.

TABLE 3

| | Estimate | Std. Error | Z value | Pr(>\|z\|) |
|---|---|---|---|---|
| Intercept | −181.375 | 61.816 | −2.934 | 0.003** |
| Weight | −1.026 | 0.365 | −2.808 | 0.005** |
| Height | 1.027 | 0.346 | 2.972 | 0.003** |
| BMI | 3.223 | 1.17 | 2.756 | 0.006** |
| Gender (male) | −2.047 | 0.967 | −2.117 | 0.034* |
| Age | 0.027 | 0.04 | 0.672 | 0.501 |
| AF Episode | −0.27 | 1.178 | −0.229 | 0.819 |
| (post-ablation) | | | | |
| AF Episode | −0.298 | 0.586 | −0.509 | 0.611 |
| (first ever) | | | | |
| AF DurInit (≥1 | −0.146 | 1.471 | −0.099 | 0.921 |
| h up to ≤24 hr) | | | | |
| Duration AF | −0.03 | 0.05 | −0.6 | 0.549 |
| CHA₂DS₂- | −0.416 | 0.44 | −0.946 | 0.344 |
| VASc Score | | | | |
| NYHA | −0.719 | 1.143 | −0.63 | 0.529 |

*p < 0.05;
**p < 0.01

Data from 81 patients (32.1% female) with a mean age of 59.8 years (range: 26.0, 84.0) were included in the analysis. This cohort had a mean weight of 87 kg (range: 57, 150), a mean height of 180 cm (range: 156, 199), and a mean BMI of 26.8 kg/m² (range: 17.2, 37.9). A logistic regression model identified height, weight, and BMI as significant predictors of cardioversion success (p<0.01) and a boundary restriction analysis revealed a negative correlation between BMI and conversion rate across the entire dataset.

Clinically significant conversion rates were observed for patients with BMI values that were considered normal (BMI<25 kg/m²⁼⁵³%; 95% CI: 36, 70), overweight (BMI≥25 and <30 kg/m²⁼⁴⁷%; 95% CI: 29, 64), and obese (BMI≥30 and <35 kg/m²⁼⁴³%; 95% CI: 17, 69); however, none of the severely obese patients (BMI≥35 mg/m²) had their AF successfully converted to sinus rhythm.

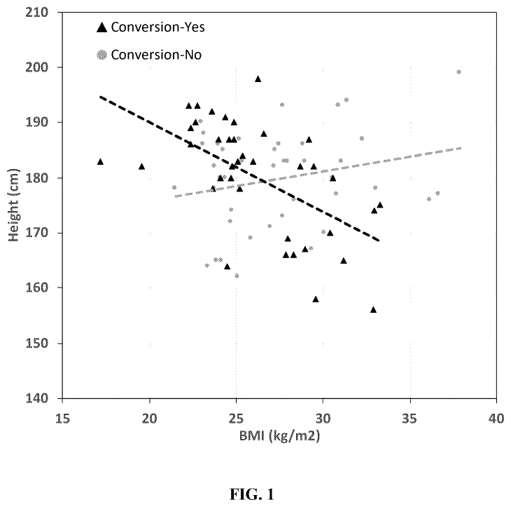

FIG. 1 is a chart showing the relationship between body mass index (BMI) and height in atrial fibrillation patients that underwent cardioversion (triangle points, r=−0.58, p<0.001, slope=−1.63) or did not undergo cardioversion (circle points, r=+0.24, p=0.132, slope=+0.52) by 90 minutes after oral inhalation of 120 mg flecainide acetate.

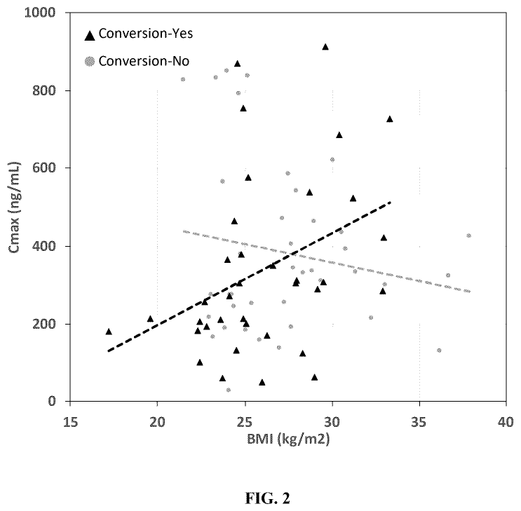

FIG. 2 is a chart showing the relationship between body mass index (BMI) and flecainide Cₘₐₓ in atrial fibrillation patients that underwent cardioversion (triangle points, r=+0.39, p=0.020, slope=+23.8) or did not undergo cardioversion (circle points, r=−0.17, p=0.290, slope=−9.4) by 90 minutes after oral inhalation of 120 mg flecainide acetate. A statistical difference (p=0.019) was observed between the patients that underwent cardioversion and the patients that did not undergo cardioversion.

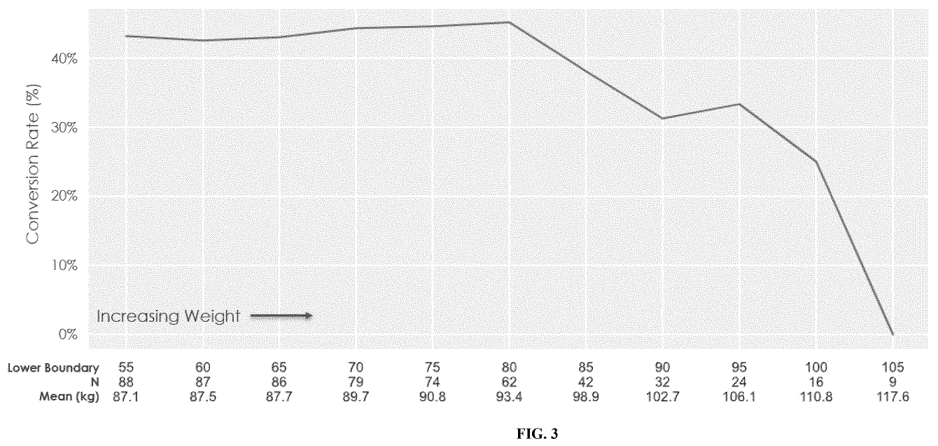

FIG. 3 is a chart depicting a boundary analysis of weight versus cardioversion rate in atrial fibrillation patients (n=88) administered 120 mg flecainide acetate via oral inhalation. The x-axis displays population with restrictions on weight lower boundary increasing in 5 kg increments from left to right. For example, a weight lower boundary of 80 kg includes all patients weighing>=80 kg. The y-axis displays conversion rate (%) for each restricted population.

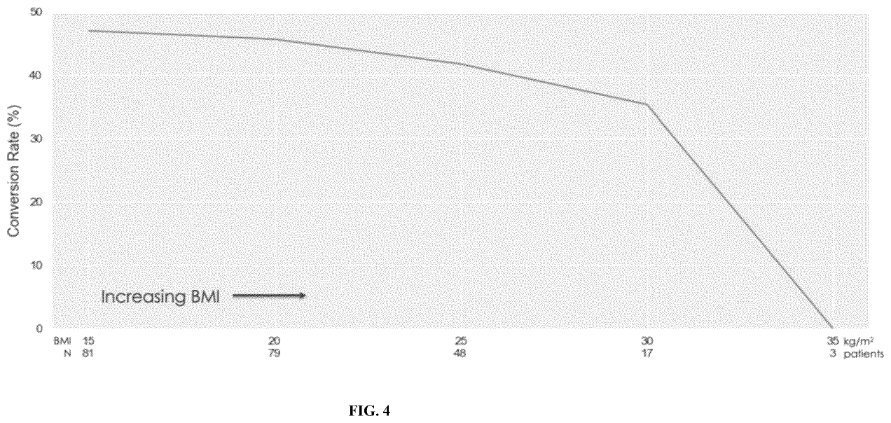

FIG. 4 is a chart depicting a boundary analysis of BMI versus cardioversion rate in atrial fibrillation patients (n=81) administered 120 mg flecainide acetate via oral inhalation. The x-axis displays population with restrictions on BMI lower boundary increasing in 5 kg/m² increments from left to right. For example, a BMI lower boundary of 15 kg/m² includes all patients with a BMI>15 kg/m². The y-axis displays conversion rate (%) for each restricted population.

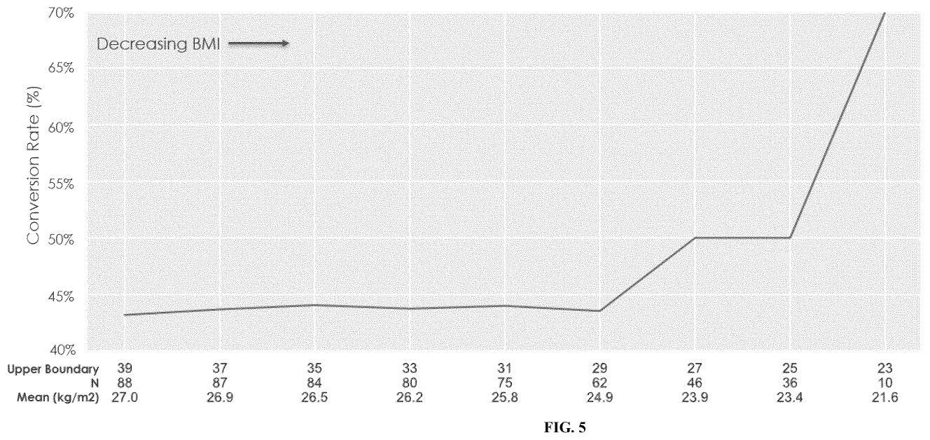

FIG. 5 is a chart depicting a boundary analysis of BMI versus cardioversion rate in atrial fibrillation patients (n=88) administered 120 mg flecainide acetate via oral inhalation. The x-axis displays population with restrictions on BMI upper boundary decreasing in 2 kg/m² increments from left to right. For example, a BMI upper boundary of 27 kg/m² includes all patients with a BMI<27 kg/m². The y-axis displays conversion rate (%) for each restricted population.

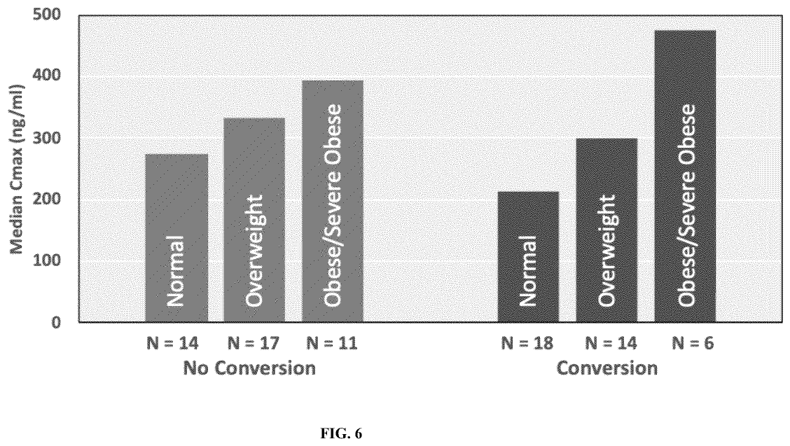

FIG. 6 is a chart depicting median Cₘₐₓ versus BMI classification (normal (BMI<25), overweight (BMI≥25 and <30), obese/severe obese (BMI≥30)) in atrial fibrillation patients (n=81) administered 120 mg flecainide acetate via oral inhalation. A positive correlation between median flecainide peak plasma concentration (Cₘₐₓ) and BMI category was observed in both the conversion and no conversion cohorts in this categorical analysis.

Successful cardioversion of recent onset AF to SR with 120 mg flecainide administered in Formulation A was attenuated in taller patients who were overweight or obese, but not in overweight or obese patients of average or short stature.

### EMBODIMENTS

The following non-limiting embodiments provide illustrative examples of the invention, but do not limit the scope of the invention.

Embodiment 1. A method of treating cardiac arrhythmia, the method comprising:
(a) identifying a subject that: (i) is suffering from said cardiac arrhythmia, and (ii) has a qualifying body mass index (BMI), wherein said qualifying BMI is a BMI of no more than about 40 kg/m²;
(b) recommending administration to said subject via oral inhalation a pharmaceutical composition that comprises a therapeutically effective amount of an antiarrhythmic agent, wherein said recommendation is made at least on the basis that said subject has said qualifying BMI; and
(c) administering to said subject said pharmaceutical composition in accordance with said recommendation.

Embodiment 2. A method of treating cardiac arrhythmia, the method comprising:
(a) identifying a subject that: (i) is suffering from said cardiac arrhythmia, and (ii) has a qualifying body mass index (BMI), wherein said qualifying BMI is a BMI of no more than about 40 kg/m²,
(b) determining that treatment of said subject is warranted at least on the basis that said subject has said qualifying BMI; and
(c) administering to said subject via oral inhalation a pharmaceutical composition that comprises a therapeutically effective amount of an antiarrhythmic agent in accordance with said determining.

Embodiment 3. A method of treating cardiac arrhythmia, the method comprising:
(a) identifying a subject that: (i) is suffering from said cardiac arrhythmia, and (ii) has a qualifying body mass index (BMI), wherein said qualifying BMI is a BMI of no more than about 40 kg/m²;
(b) designating said subject as eligible for treatment at least on the basis that said subject has said qualifying BMI;
(c) administering to said subject via oral inhalation a pharmaceutical composition that comprises a therapeutically effective amount of an antiarrhythmic agent in accordance with said designating.

Embodiment 4. The method of any one of embodiments 1-3, wherein said subject has a systolic blood pressure that is greater than about 90 mmHg at initiation of said administering.

Embodiment 5. The method of any one of embodiments 1-3, wherein said subject has a systolic blood pressure that is from about 100 mmHg to about 160 mmHg at initiation of said administering.

Embodiment 6. The method of any one of embodiments 1-5, wherein said subject has a ventricular rate that is no more than 170 BPM at initiation of said administering.

Embodiment 7. The method of any one of embodiments 1-5, wherein said subject has a ventricular rate that is from about 80 BPM to about 155 BPM at initiation of said administering.

Embodiment 8. The method of any one of embodiments 1-7, wherein said qualifying BMI is a BMI of no more than about 37 kg/m², about 35 kg/m², about 33 kg/m², about 31 kg/m², about 30 kg/m², about 29 kg/m², about 27 kg/m², or about 25 kg/m².

Embodiment 9. The method of any one of embodiments 1-7, wherein said qualifying BMI is a BMI of no more than about 35 kg/m².

Embodiment 10. The method of any one of embodiments 1-7, wherein said qualifying BMI is a BMI of no more than about 33 kg/m².

Embodiment 11. The method of any one of embodiments 1-10, wherein said antiarrhythmic agent is a class I, II, III, IV, or V antiarrhythmic agent.

Embodiment 12. The method of any one of embodiments 1-10, wherein said pharmaceutical composition comprises a therapeutically effective amount of flecainide or a pharmaceutically acceptable salt thereof.

Embodiment 13. The method of embodiment 12, wherein said pharmaceutical composition in the form of a liquid solution.

Embodiment 14. The method of embodiment 12, wherein said pharmaceutical composition has said salt of flecainide at a concentration that is at least 60 mg/mL.

Embodiment 15. The method of any one of embodiments 12-14, wherein said pharmaceutical composition further comprises a cyclodextrin.

Embodiment 16. The method of embodiment 15, wherein said cyclodextrin comprises hydroxypropyl-β-cyclodextrin.

Embodiment 17. The method of embodiment 15 or 16, wherein said concentration of said cyclodextrin is from about 15% (w/v) to about 25% (w/v) of said solution.

Embodiment 18. The method of embodiment 15 or 16, wherein said concentration of said cyclodextrin is at least about 10% (w/v) of said solution.

Embodiment 19. The method of embodiment 15 or 16, wherein said concentration of said cyclodextrin is about 20% (w/v) of said solution.

Embodiment 20. The method of any one of embodiments 12-19, wherein a pH of said solution is above 5.5 at room temperature.

Embodiment 21. The method of any one of embodiments 12-20, wherein said concentration of said salt of flecainide is about 65 mg/mL to about 130 mg/mL.

Embodiment 22. The method of any one of embodiments 12-20, wherein said concentration of said salt of flecainide is about 75 mg/mL.

Embodiment 23. The method of any one of embodiments 12-22, wherein said salt of flecainide is selected from the group consisting of: flecainide acetate, flecainide hydrochloride, flecainide citrate, flecainide phosphate, and flecainide nitrate.

Embodiment 24. The method of any one of embodiments 12-22, wherein said salt of flecainide comprises flecainide acetate.

Embodiment 25. The method of any one of embodiments 12-24, wherein said pharmaceutical composition further comprises an acid.

Embodiment 26. The method of embodiment 25, wherein said acid is selected from the group consisting of: acetic acid, citric acid, nitric acid, hydrochloric acid, sulfuric acid, maleic acid, tartaric acid, phosphoric acid, aconitic acid, adipic acid, ascorbic acid, benzoic acid, caprylic acid, cholic acid, formic acid, glutamic acid, lactic acid, propionic acid, sorbic acid, stearic acid, and succinic acid.

Embodiment 27. The method of embodiment 25 or 26, wherein a concentration of said acid in said pharmaceutical composition is about 2 mM to about 50 mM.

Embodiment 28. The method of embodiment 25 or 26, wherein said concentration of said acid is about 20 mM.

Embodiment 29. The method of embodiment 25 or 26, wherein said concentration of said acid is about 5 mM.

Embodiment 30. The method of any one of embodiments 25-29, wherein said acid comprises acetic acid.

Embodiment 31. The method of embodiment 30, wherein a concentration of acetic acid is about 5 mM.

Embodiment 32. The method of any one of embodiments 25-31, wherein said acid comprises a mixture of acids selected from the group consisting of: acetic acid, citric acid, nitric acid, hydrochloric acid, and sulfuric acid.

Embodiment 33. The method of any one of embodiments 20-32, wherein said pH of said solution is from about 5.5 to about 6.5.

Embodiment 34. The method of any one of embodiments 20-32, wherein said pH of said solution is about 5.9.

Embodiment 35. The method of any one of embodiments 12-34, wherein said pharmaceutical composition further comprises a sweetener.

Embodiment 36. The method of embodiment 35, wherein said sweetener comprises saccharin.

Embodiment 37. The method of embodiment 35, wherein said sweetener comprises saccharin sodium.

Embodiment 38. The method of any one of embodiments 12-37, wherein said administration results in a peak plasma concentration (Cₘₐₓ) of said salt of flecainide in said subject that is at least 200 ng/ml.

Embodiment 39. The method of embodiment 38, wherein said Cₘₐₓ is between about 250 ng/ml and about 1000 ng/ml.

Embodiment 40. The method of embodiment 38, wherein said Cₘₐₓ is between about 300 ng/ml and about 700 ng/ml.

Embodiment 41. The method of any one of embodiments 12-40, wherein about 100 mg to about 250 mg of said salt of flecainide is administered to said subject via inhalation.

Embodiment 42. The method of any one of embodiments 12-40, wherein about 90 mg of said salt of flecainide is administered to said subject via inhalation.

Embodiment 43. The method of any one of embodiments 12-40, wherein about 120 mg of said salt of flecainide is administered to said subject via inhalation.

Embodiment 44. The method of any one of embodiments 12-40, wherein about 200 mg of said salt of flecainide is administered to said subject via inhalation.

Embodiment 45. The method of any one of embodiments 1-44, wherein said administration of said pharmaceutical composition is performed within about 10 min.

Embodiment 46. The method of any one of embodiments 1-45, wherein said pharmaceutical composition is a nebulized solution that comprises nebulized droplets having a mass median aerodynamic diameter of less than 10 μm.

Embodiment 47. The method of any one of embodiments 1-40, wherein said administration of said pharmaceutical composition is performed via one or two inhalations.

Embodiment 48. The method of any one of embodiments 1-40, wherein said administration of said pharmaceutical composition is performed via two inhalations that are separated by a break for from about 10 seconds to about 1 minute.

Embodiment 49. The method of any one of embodiments 1-44, wherein said administration of said pharmaceutical composition is performed within about 5 min.

Embodiment 50. The method of any one of embodiments 1-49, wherein said administration is performed via a nebulizer.

Embodiment 51. The method of embodiment 50, wherein said nebulizer is a breath-actuated nebulizer.

Embodiment 52. The method of embodiment 50, wherein said nebulizer is a jet nebulizer.

Embodiment 53. The method of embodiment 50, wherein said nebulizer is a vibrating mesh nebulizer.

Embodiment 54. The method of any one of embodiments 1-53, wherein said cardiac arrhythmia comprises atrial arrhythmia.

Embodiment 55. The method of embodiment 54, wherein said atrial arrhythmia comprises tachycardia.

Embodiment 56. The method of embodiment 54 or 55, comprising acute treatment after detection of said atrial arrhythmia in said subject.

Embodiment 57. The method of any one of embodiments 1-56, wherein said cardiac arrhythmia comprises atrial fibrillation.

Embodiment 58. The method of embodiment 57, wherein said atrial fibrillation is recurrent atrial fibrillation.

Embodiment 59. The method of embodiment 57, wherein said atrial fibrillation is paroxysmal atrial fibrillation.

Embodiment 60. A method of treating cardiac arrhythmia, the method comprising:
(a) identifying a subject that: (i) is suffering from said cardiac arrhythmia, and (ii) has a qualifying body mass index (BMI), wherein said qualifying BMI is a BMI of at least about 23 kg/m²;
(b) administering to said subject via oral inhalation a first dose of a pharmaceutical composition comprising an antiarrhythmic agent;
(c) after said administering of (b), waiting for a period of time, wherein said period of time is at least about 10 minutes;
(d) after (c), determining that said subject exhibits said cardiac arrhythmia after said period of time;
(e) after (d), recommending administration of a second dose of said pharmaceutical composition via oral inhalation to said subject, wherein said recommending is made based on at least: (i) said determining that subject exhibits said cardiac arrhythmia after said period of time; and (ii) that subject has said qualifying BMI; and
(f) administering said second dose of said pharmaceutical composition via oral inhalation to said subject in accordance with said recommendation.

Embodiment 61. A method of treating cardiac arrhythmia, the method comprising:
(a) identifying a subject that: (i) is suffering from said cardiac arrhythmia, and (ii) has a qualifying body mass index (BMI), wherein said qualifying BMI is a BMI of at least about 23 kg/m²;
(b) administering to said subject via oral inhalation a first dose of a pharmaceutical composition comprising an antiarrhythmic agent;
(c) after the administering of (b), waiting for a period of time, wherein said period of time is at least about 10 minutes;
(d) after (c), determining that said subject exhibits said cardiac arrhythmia after said period of time;
(e) after the waiting, determining that administration of a second dose of said pharmaceutical composition via oral inhalation to said subject is warranted based on at least: (i) said determining that subject exhibits said cardiac arrhythmia after said period of time; and (ii) that subject has said qualifying BMI; and
(f) administering said second dose of said pharmaceutical composition via oral inhalation to said subject in accordance with said determining that administration of said second dose is warranted.

Embodiment 62. A method of treating cardiac arrhythmia, the method comprising:
(a) identifying a subject that is: (i) suffering from said cardiac arrhythmia, and (ii) has a qualifying body mass index (BMI), wherein said qualifying BMI is a BMI of at least about 23 kg/m²;
(b) administering to said subject via oral inhalation a first dose of a pharmaceutical composition comprising an antiarrhythmic agent;
(c) after the administering of (b), waiting for a period of time, wherein said period of time is at least about 10 minutes,
(d) after (c), determining that said subject exhibits said cardiac arrhythmia after said period of time
(e) after the waiting, designating said subject as eligible for treatment with a second dose of said pharmaceutical composition via oral inhalation, wherein said designating is made based on at least: (i) said determining that subject exhibits said cardiac arrhythmia after said period of time; and (ii) that subject has said qualifying BMI; administering to said subject via oral inhalation said second dose of said (f) pharmaceutical composition in accordance with said designating.

Embodiment 63. The method of any one of embodiments 60-62, wherein said determining of (d) comprises obtaining an ECG of said subject.

Embodiment 64. The method of any one of embodiments 60-63, wherein said period of time is at least about 20 minutes, at least about 30 minutes, at least about 40 minutes, at least about 50 minutes, at least about 60 minutes, at least about 70 minutes, at least about 80 minutes, at least about 90 minutes, at least about 100 minutes, at least about 110 minutes, or at least about 120 minutes.

Embodiment 65. The method of any one of embodiments 60-63, wherein said period of time is at least about 20 minutes.

Embodiment 66. The method of any one of embodiments 60-65, wherein said qualifying BMI is a BMI of at least about 25 kg/m², 27 kg/m², about 29 kg/m², about 30 kg/m², about 31 kg/m², about 33 kg/m², about 35 kg/m², about 37 kg/m², or about 40 kg/m².

Embodiment 67. The method of any one of embodiments 60-65, wherein said qualifying BMI is a BMI of at least about 25 kg/m².

Embodiment 68. The method of any one of embodiments 60-65, wherein said qualifying BMI is a BMI from about 23 kg/m² to about 40 kg/m².

Embodiment 69. The method of any one of embodiments 60-65, wherein said qualifying BMI is a BMI from about 23 kg/m² to about 37 kg/m².

Embodiment 70. The method of any one of embodiments 60-65, wherein said qualifying BMI is a BMI from about 23 kg/m² to about 35 kg/m².

Embodiment 71. The method of any one of embodiments 60-65, wherein said qualifying BMI is a BMI from about 25 kg/m² to about 40 kg/m².

Embodiment 72. The method of any one of embodiments 60-65, wherein said qualifying BMI is a BMI from about 25 kg/m² to about 37 kg/m².

Embodiment 73. The method of any one of embodiments 60-65, wherein said qualifying BMI is a BMI from about 25 kg/m² to about 35 kg/m².

Embodiment 74. The method of any one of embodiments 60-73, wherein said identifying of (a) further comprises determining said BMI of said subject.

Embodiment 75. The method of embodiment 74, wherein said determining said BMI comprises determining subject height and body mass.

Embodiment 76. The method of embodiment 75, wherein said subject height or body mass is reported by said subject.

Embodiment 77. The method of embodiment 75 or 76, wherein said subject height or body mass is measured by a health practitioner.

Embodiment 78. The method of any one of embodiments 60-77, wherein said subject has a systolic blood pressure that is greater than about 90 mmHg at the time of treating.

Embodiment 79. The method of any one of embodiments 60-77, wherein said subject has a systolic blood pressure that is from about 100 mmHg to about 160 mmHg at the time of treating.

Embodiment 80. The method of any one of embodiments 60-79, wherein said subject has a ventricular rate that is no more than 170 BPM at the time of treating.

Embodiment 81. The method of any one of embodiments 60-79, wherein said subject has a ventricular rate that is from about 80 BPM to about 155 BPM at the time of treating.

Embodiment 82. The method of any one of embodiments 60-81, wherein said antiarrhythmic agent is a class I, II, III, IV, or V antiarrhythmic agent.

Embodiment 83. The method of any one of embodiments 60-81, wherein said pharmaceutical composition comprises a therapeutically effective amount of flecainide or a pharmaceutically acceptable salt thereof.

Embodiment 84. The method of embodiment 83, wherein said pharmaceutical composition in the form of a liquid solution.

Embodiment 85. The method of embodiment 84, wherein said pharmaceutical composition has said salt of flecainide at a concentration that is at least 60 mg/mL.

Embodiment 86. The method of any one of embodiments 83-85, wherein said pharmaceutical composition further comprises a cyclodextrin.

Embodiment 87. The method of embodiment 86, wherein said cyclodextrin comprises hydroxypropyl-β-cyclodextrin.

Embodiment 88. The method of embodiment 86 or 87, wherein said concentration of said cyclodextrin is from about 15% (w/v) to about 25% (w/v) of said solution.

Embodiment 89. The method of embodiment 86 or 87, wherein said concentration of said cyclodextrin is at least about 10% (w/v) of said solution.

Embodiment 90. The method of embodiment 86 or 87, wherein said concentration of said cyclodextrin is about 20% (w/v) of said solution.

Embodiment 91. The method of any one of embodiments 85-90, wherein a pH of said solution is above 5.5 at room temperature.

Embodiment 92. The method of any one of embodiments 85-91, wherein said concentration of said salt of flecainide is about 65 mg/mL to about 130 mg/mL.

Embodiment 93. The method of any one of embodiments 85-91, wherein said concentration of said salt of flecainide is about 75 mg/mL.

Embodiment 94. The method of any one of embodiments 85-93, wherein said salt of flecainide is selected from the group consisting of: flecainide acetate, flecainide hydrochloride, flecainide citrate, flecainide phosphate, and flecainide nitrate.

Embodiment 95. The method of any one of embodiments 85-93, wherein said salt of flecainide comprises flecainide acetate.

Embodiment 96. The method of any one of embodiments 83-95, wherein said pharmaceutical composition further comprises an acid.

Embodiment 97. The method of embodiment 96, wherein said acid is selected from the group consisting of: acetic acid, citric acid, nitric acid, hydrochloric acid, sulfuric acid, maleic acid, tartaric acid, phosphoric acid, aconitic acid, adipic acid, ascorbic acid, benzoic acid, caprylic acid, cholic acid, formic acid, glutamic acid, lactic acid, propionic acid, sorbic acid, stearic acid, and succinic acid.

Embodiment 98. The method of embodiment 96 or 97, wherein a concentration of said acid in said pharmaceutical composition is about 2 mM to about 50 mM.

Embodiment 99. The method of embodiment 96 or 97, wherein said concentration of said acid is about 20 mM.

Embodiment 100. The method of embodiment 96 or 97, wherein said concentration of said acid is about 5 mM.

Embodiment 101. The method of any one of embodiments 96-100, wherein said acid comprises acetic acid.

Embodiment 102. The method of embodiment 101, wherein a concentration of acetic acid is about 5 mM.

Embodiment 103. The method of any one of embodiments 96-102, wherein said acid comprises a mixture of acids selected from the group consisting of: acetic acid, citric acid, nitric acid, hydrochloric acid, and sulfuric acid.

Embodiment 104. The method of any one of embodiments 91-103, wherein said pH of said solution is from about 5.5 to about 6.5.

Embodiment 105. The method of any one of embodiments 91-103, wherein said pH of said solution is about 5.9.

Embodiment 106. The method of any one of embodiments 83-105, wherein said pharmaceutical composition further comprises a sweetener.

Embodiment 107. The method of embodiment 106, wherein said sweetener comprises saccharin.

Embodiment 108. The method of embodiment 106, wherein said sweetener comprises saccharin sodium.

Embodiment 109. The method of any one of embodiments 83-108, wherein administration of said first dose results in a peak plasma concentration (Cₘₐₓ) of said salt of flecainide in said subject that is at least 200 ng/ml.

Embodiment 110. The method of embodiment 109, wherein said Cₘₐₓ is between about 250 ng/ml and about 1000 ng/ml.

Embodiment 111. The method of embodiment 109, wherein said Cₘₐₓ is between about 300 ng/ml and about 700 ng/ml.

Embodiment 112. The method of any one of embodiments 83-112, wherein said second dose comprises said salt of flecainide in an amount that is equivalent to about 40% to about 80% (w/w) of said first dose.

Embodiment 113. The method of any one of embodiments 83-112, wherein said second dose comprises said salt of flecainide in an amount that is equivalent to said first dose.

Embodiment 114. The method of any one of embodiments 83-112, wherein said second dose comprises said salt of flecainide in an amount that is equivalent to about 40% to about 60% (w/w) of said first dose.

Embodiment 115. The method of any one of embodiments 83-112, wherein said second dose comprises said salt of flecainide in an amount that is equivalent to about 50% (w/w) of said first dose.

Embodiment 116. The method of any one of embodiments 83-115, wherein said first dose comprises from about 100 mg to about 250 mg said salt of flecainide.

Embodiment 117. The method of any one of embodiments 83-115, wherein said first dose comprises from about 100 mg to about 140 mg said salt of flecainide.

Embodiment 118. The method of any one of embodiments 83-115, wherein said first dose comprises about 120 mg said salt of flecainide.

Embodiment 119. The method of any one of embodiments 83-115, wherein said second dose comprises from about 30 mg to about 90 mg said salt of flecainide.

Embodiment 120. The method of any one of embodiments 83-115, wherein said second dose comprises from about 50 mg to about 70 mg said salt of flecainide.

Embodiment 121. A method of treating cardiac arrhythmia in a subject, wherein the method comprises:
(a) administering to said subject via oral inhalation a first dose of a pharmaceutical composition, wherein said pharmaceutical composition is an aqueous solution that comprises flecainide or a pharmaceutically acceptable salt thereof at a concentration that is at least 60 mg/ml;
(b) after the administering of (a), waiting for a period of time, wherein said period of time is at least about 10 minutes; and
(c) after the waiting, administering to said subject a second dose of said pharmaceutical composition via oral inhalation, wherein said second dose comprises said flecainide or salt thereof in an amount that is equivalent to about 40% to about 100% (w/w) of said first dose.

Embodiment 122. A method of treating cardiac arrhythmia in a subject, wherein the method comprises:
(a) administering to said subject via oral inhalation a first dose of a pharmaceutical composition comprising from about 80 mg to about 160 mg flecainide or a pharmaceutically acceptable salt thereof, wherein said pharmaceutical composition is an aqueous solution of said flecainide or salt thereof;
(b) after the administering of (a), waiting for a period of time, wherein said period of time is at least about 10 minutes; and
(c) after the waiting, administering to said subject a second dose of said pharmaceutical composition via oral inhalation, wherein said second dose comprises said flecainide or salt thereof in an amount that is equivalent to about 40% to about 100% (w/w) of said first dose.

Embodiment 123. The method of embodiment 121 or 122, wherein said subject is obese.

Embodiment 124. The method of embodiment 121 or 122, wherein said subject has a body mass index (BMI) of at least 23 kg/m².

Embodiment 125. The method of embodiment 121 or 122, wherein said subject has a body mass index (BMI) of at least 25 kg/m².

Embodiment 126. The method of embodiment 121 or 122, wherein said subject has a body mass index (BMI) of at least 27 kg/m².

Embodiment 127. The method of embodiment 121 or 122, wherein said subject has a body mass index (BMI) of at least 30 kg/m².

Embodiment 128. The method of embodiment 121 or 122, wherein said subject has a body mass index (BMI) that is from about 23 kg/m² to about 40 kg/m².

Embodiment 129. The method of embodiment 121 or 122, wherein said subject has a body mass index (BMI) that is from about 23 kg/m² to about 37 kg/m².

Embodiment 130. The method of embodiment 121 or 122, wherein said subject has a body mass index (BMI) that is from about 23 kg/m² to about 35 kg/m².

Embodiment 131. The method of embodiment 121 or 122, wherein said subject has a body mass index (BMI) that is from about 25 kg/m² to about 40 kg/m².

Embodiment 132. The method of embodiment 121 or 122, wherein said subject has a body mass index (BMI) that is from about 25 kg/m² to about 37 kg/m².

Embodiment 133. The method of embodiment 121 or 122, wherein said subject has a body mass index (BMI) that is from about 25 kg/m² to about 35 kg/m².

Embodiment 134. The method of any one of embodiments 121-133, wherein said period of time is about 20 minutes, about 30 minutes, about 40 minutes, about 50 minutes, about 60 minutes, about 70 minutes, about 80 minutes, about 90 minutes, or about 100 minutes.

Embodiment 135. The method of any one of embodiments 121-133, wherein said period of time is about 20 minutes.

Embodiment 136. The method of any one of embodiments 121-135, wherein said second dose comprises said flecainide in an amount that is equivalent to about 40% to about 80% (w/w) of said first dose.

Embodiment 137. The method of any one of embodiments 121-135, wherein said second dose comprises said flecainide in an amount that is equivalent to said first dose.

Embodiment 138. The method of any one of embodiments 121-135, wherein said second dose comprises said flecainide in an amount that equivalent to about 40% to about 60% (w/w) of said first dose.

Embodiment 139. The method of any one of embodiments 121-135, wherein said second dose comprises said flecainide in an amount that equivalent to about 50% (w/w) of said first dose.

Embodiment 140. The method of any one of embodiments 121-135, wherein said first dose comprises about 120 mg flecainide acetate, and said second dose comprises about 60 mg flecainide acetate.

Embodiment 141. The method of any one of embodiments 60-81 and 121-140, wherein said administering said first dose comprises:
(i) inhaling an aerosol of said pharmaceutical composition via a nebulizer for a first duration that is from about 3 minutes to about 4 minutes;
(ii) after said first duration, pausing inhalation for a second duration that is from about 30 seconds to about 90 seconds; and
(iii) after said second duration, resuming inhalation of said aerosol of said pharmaceutical composition via said nebulizer for a third duration that is from about 3 minutes to about 4 minutes, thereby administering said first dose.

Embodiment 142. The method of embodiment 141, wherein said first duration is about

3.5 minutes.

Embodiment 143. The method of embodiment 141 or 142, wherein said second duration is about 1 minute.

Embodiment 144. The method of any one of embodiments 141-143, wherein said third duration is about 3.5 minutes.

Embodiment 145. The method of any one of embodiments 60-81 and 121-144, wherein said administering said second dose comprises inhaling an aerosol of said pharmaceutical composition via a nebulizer for about 3 minutes to about 4 minutes, thereby administering said second dose.

Embodiment 146. The method of any one of embodiments 60-81 and 121-144, wherein said administering said second dose comprises inhaling an aerosol of said pharmaceutical composition via a nebulizer for about 3.5 minutes, thereby administering said second dose.

Embodiment 147. The method of any one of embodiments 141-146, wherein said subject inhales said aerosol via tidal breathing.

Embodiment 148. The method of any one of embodiments 141-147, wherein said nebulizer is a breath-actuated nebulizer.

Embodiment 149. The method of any one of embodiments 141-147, wherein said nebulizer is a jet nebulizer.

Embodiment 150. The method of any one of embodiments 141-147, wherein said nebulizer is a vibrating mesh nebulizer.

Embodiment 151. The method of any one of embodiments 141-150, wherein said aerosol comprises nebulized droplets of said pharmaceutical composition, wherein said nebulized droplets have a mass median aerodynamic diameter of less than 10 μm.

Embodiment 152. The method of any one of embodiments 141-151, wherein said pharmaceutical composition has said salt of flecainide at a concentration that is at least 60 mg/mL.

Embodiment 153. The method of any one of embodiments 141-152, wherein said pharmaceutical composition further comprises a cyclodextrin.

Embodiment 154. The method of embodiment 153, wherein said cyclodextrin comprises hydroxypropyl-β-cyclodextrin.

Embodiment 155. The method of embodiment 153 or 154, wherein said concentration of said cyclodextrin is from about 15% (w/v) to about 25% (w/v) of said solution.

Embodiment 156. The method of embodiment 153 or 154, wherein said concentration of said cyclodextrin is at least about 10% (w/v) of said solution.

Embodiment 157. The method of embodiment 153 or 154, wherein said concentration of said cyclodextrin is about 20% (w/v) of said solution.

Embodiment 158. The method of any one of embodiments 152-157, wherein a pH of said solution is above 5.5 at room temperature.

Embodiment 159. The method of any one of embodiments 152-158, wherein said concentration of said salt of flecainide is about 65 mg/mL to about 130 mg/mL.

Embodiment 160. The method of any one of embodiments 152-158, wherein said concentration of said salt of flecainide is about 75 mg/mL.

Embodiment 161. The method of any one of embodiments 152-159, wherein said salt of flecainide is selected from the group consisting of: flecainide acetate, flecainide hydrochloride, flecainide citrate, flecainide phosphate, and flecainide nitrate.

Embodiment 162. The method of any one of embodiments 152-159, wherein said salt of flecainide comprises flecainide acetate.

Embodiment 163. The method of any one of embodiments 152-160, wherein said pharmaceutical composition further comprises an acid.

Embodiment 164. The method of embodiment 163, wherein said acid is selected from the group consisting of: acetic acid, citric acid, nitric acid, hydrochloric acid, sulfuric acid, maleic acid, tartaric acid, phosphoric acid, aconitic acid, adipic acid, ascorbic acid, benzoic acid, caprylic acid, cholic acid, formic acid, glutamic acid, lactic acid, propionic acid, sorbic acid, stearic acid, and succinic acid.

Embodiment 165. The method of embodiment 163 or 164, wherein a concentration of said acid in said pharmaceutical composition is about 2 mM to about 50 mM.

Embodiment 166. The method of embodiment 163 or 164, wherein said concentration of said acid is about 20 mM.

Embodiment 167. The method of embodiment 163 or 164, wherein said concentration of said acid is about 5 mM.

Embodiment 168. The method of any one of embodiments 163-167, wherein said acid comprises acetic acid.

Embodiment 169. The method of embodiment 168, wherein a concentration of acetic acid is about 5 mM.

Embodiment 170. The method of any one of embodiments 163-169, wherein said acid comprises a mixture of acids selected from the group consisting of: acetic acid, citric acid, nitric acid, hydrochloric acid, and sulfuric acid.

Embodiment 171. The method of any one of embodiments 152-170, wherein said pH of said solution is from about 5.5 to about 6.5.

Embodiment 172. The method of any one of embodiments 152-170, wherein said pH of said solution is about 5.9.

Embodiment 173. The method of any one of embodiments 152-172, wherein said pharmaceutical composition further comprises a sweetener.

Embodiment 174. The method of embodiment 173, wherein said sweetener comprises saccharin.

Embodiment 175. The method of embodiment 173, wherein said sweetener comprises saccharin sodium.

Embodiment 176. The method of any one of embodiments 60-175, wherein said cardiac arrhythmia comprises atrial arrhythmia.

Embodiment 177. The method of embodiment 176, wherein said atrial arrhythmia comprises tachycardia.

Embodiment 178. The method of any one of embodiments 60-175, wherein said cardiac arrhythmia comprises atrial fibrillation.

Embodiment 179. The method of embodiment 178, wherein said atrial fibrillation is recurrent atrial fibrillation.

Embodiment 180. The method of embodiment 178, wherein said atrial fibrillation is paroxysmal atrial fibrillation.

Embodiment 181. A kit comprising a first dose and a second dose of a pharmaceutical composition formulated for oral inhalation, wherein:
(a) said pharmaceutical composition comprises a class I antiarrhythmic agent or a pharmaceutically acceptable salt thereof;
(b) said second dose comprises said class I antiarrhythmic agent or a pharmaceutically acceptable salt thereof in an amount that is equivalent to about 40% to about 80% (w/w) of said first dose;
(c) said first dose is provided in a first vessel, and said second dose is provided in a second vessel.

Embodiment 182. The kit of embodiment 181, wherein said antiarrhythmic agent is flecainide or a pharmaceutically acceptable salt thereof.

Embodiment 183. The kit of embodiment 181 or embodiment 182, wherein said pharmaceutical composition in the form of a liquid solution.

Embodiment 184. The kit of embodiment 183, wherein said pharmaceutical composition has said salt of flecainide at a concentration that is at least 60 mg/mL.

Embodiment 185. The kit of any one of embodiments 183-184, wherein said pharmaceutical composition further comprises a cyclodextrin.

Embodiment 186. The kit of embodiment 185, wherein said cyclodextrin comprises hydroxypropyl-β-cyclodextrin.

Embodiment 187. The kit of embodiment 185 or 186, wherein said concentration of said cyclodextrin is from about 15% (w/v) to about 25% (w/v) of said solution.

Embodiment 188. The kit of embodiment 185 or 186, wherein said concentration of said cyclodextrin is at least about 10% (w/v) of said solution.

Embodiment 189. The kit of embodiment 185 or 186, wherein said concentration of said cyclodextrin is about 20% (w/v) of said solution.

Embodiment 190. The kit of any one of embodiments 184-189, wherein a pH of said solution is above 5.5 at room temperature.

Embodiment 191. The kit of any one of embodiments 184-190, wherein said concentration of said salt of flecainide is about 65 mg/mL to about 130 mg/mL.

Embodiment 192. The kit of any one of embodiments 184-190, wherein said concentration of said salt of flecainide is about 75 mg/mL.

Embodiment 193. The kit of any one of embodiments 184-192, wherein said salt of flecainide is selected from the group consisting of: flecainide acetate, flecainide hydrochloride, flecainide citrate, flecainide phosphate, and flecainide nitrate.

Embodiment 194. The kit of any one of embodiments 184-192, wherein said salt of flecainide comprises flecainide acetate.

Embodiment 195. The kit of any one of embodiments 184-194, wherein said pharmaceutical composition further comprises an acid.

Embodiment 196. The kit of embodiment 195, wherein said acid is selected from the group consisting of: acetic acid, citric acid, nitric acid, hydrochloric acid, sulfuric acid, maleic acid, tartaric acid, phosphoric acid, aconitic acid, adipic acid, ascorbic acid, benzoic acid, caprylic acid, cholic acid, formic acid, glutamic acid, lactic acid, propionic acid, sorbic acid, stearic acid, and succinic acid.

Embodiment 197. The kit of embodiment 195 or 196, wherein a concentration of said acid in said pharmaceutical composition is about 2 mM to about 50 mM.

Embodiment 198. The kit of embodiment 195 or 196, wherein said concentration of said acid is about 20 mM.

Embodiment 199. The kit of embodiment 195 or 196, wherein said concentration of said acid is about 5 mM.

Embodiment 200. The kit of any one of embodiments 195-199, wherein said acid comprises acetic acid.

Embodiment 201. The kit of embodiment 200, wherein a concentration of acetic acid is about 5 mM.

Embodiment 202. The kit of any one of embodiments 195-201, wherein said acid comprises a mixture of acids selected from the group consisting of: acetic acid, citric acid, nitric acid, hydrochloric acid, and sulfuric acid.

Embodiment 203. The kit of any one of embodiments 184-202, wherein said pH of said solution is from about 5.5 to about 6.5.

Embodiment 204. The kit of any one of embodiments 184-202, wherein said pH of said solution is about 5.9.

Embodiment 205. The kit of any one of embodiments 184-204, wherein said pharmaceutical composition further comprises a sweetener.

Embodiment 206. The kit of embodiment 205, wherein said sweetener comprises saccharin.

Embodiment 207. The kit of embodiment 205, wherein said sweetener comprises saccharin sodium.

Embodiment 208. The kit of any one of embodiments 184-207, wherein said second dose comprises said salt of flecainide in an amount that is equivalent to about 40% to about 80% (w/w) of said first dose.

Embodiment 209. The kit of any one of embodiments 184-207, wherein said second dose comprises said salt of flecainide in an amount that is equivalent to said first dose.

Embodiment 210. The kit of any one of embodiments 184-207, wherein said second dose comprises said salt of flecainide in an amount that is equivalent to about 40% to about 60% (w/w) of said first dose.

Embodiment 211. The kit of any one of embodiments 184-207, wherein said second dose comprises said salt of flecainide in an amount that is equivalent to about 50% (w/w) of said first dose.

Embodiment 212. The kit of any one of embodiments 184-211, wherein said first dose comprises from about 100 mg to about 250 mg said salt of flecainide.

Embodiment 213. The kit of any one of embodiments 184-211, wherein said first dose comprises from about 100 mg to about 140 mg said salt of flecainide.

Embodiment 214. The kit of any one of embodiments 184-211, wherein said first dose comprises about 120 mg said salt of flecainide.

Embodiment 215. The kit of any one of embodiments 184-211, wherein said second dose comprises from about 30 mg to about 90 mg said salt of flecainide.

Embodiment 216. The kit of any one of embodiments 184-211, wherein said second dose comprises from about 50 mg to about 70 mg said salt of flecainide.

Embodiment 217. The kit of any one of embodiments 184-207, wherein said first dose comprises about 120 mg flecainide acetate, and said second dose comprises about 60 mg flecainide acetate.

Embodiment 218. The kit of any one of embodiments 181-217, wherein said kit further comprises a nebulizer.

Embodiment 219. The kit of embodiment 218, wherein said nebulizer is a breath-actuated nebulizer.

Embodiment 220. The kit of embodiment 218, wherein said nebulizer is a jet nebulizer.

Embodiment 221. The kit of embodiment 218, wherein said nebulizer is a vibrating mesh nebulizer.

Embodiment 222. The kit of any one of embodiments 181-221, wherein the kit further comprises instructions for administration of said first dose and said second dose in accordance with the method of any one of embodiments 60-180.

Embodiment 223. A kit according to any one of embodiments 181-221 for use in treating a patient suffering from atrial fibrillation, wherein said patient has a qualifying BMI, wherein said qualifying BMI is a BMI that is at least 23 kg/m².

Embodiment 224. The kit for use according to embodiment 223, wherein said qualifying BMI is a BMI that is at least 25 kg/m².

Embodiment 225. The kit for use according to embodiment 223, wherein said qualifying BMI is a BMI that is at least 27 kg/m².

Embodiment 226. The kit for use according to embodiment 223, wherein said qualifying BMI is a BMI that is at least 29 kg/m².

Embodiment 227. The kit for use according to embodiment 223, wherein said qualifying BMI is a BMI that is at least 30 kg/m².

Embodiment 228. The kit for use according to embodiment 223, wherein said qualifying BMI is a BMI that is from about 23 kg/m² to about 40 kg/m².

Embodiment 229. The kit for use according to embodiment 223, wherein said qualifying BMI is a BMI that is from about 23 kg/m² to about 37 kg/m².

Embodiment 230. The kit for use according to embodiment 223, wherein said qualifying BMI is a BMI that is from about 23 kg/m² to about 35 kg/m².

Embodiment 231. The kit for use according to embodiment 223, wherein said qualifying BMI is a BMI that is from about 25 kg/m² to about 40 kg/m².

Embodiment 232. The kit for use according to embodiment 223, wherein said qualifying BMI is a BMI that is from about 25 kg/m² to about 37 kg/m².

Embodiment 233. The kit for use according to embodiment 223, wherein said qualifying BMI is a BMI that is from about 25 kg/m² to about 35 kg/m².

Embodiment 234. A pharmaceutical composition comprising a therapeutically effective amount of an antiarrhythmic agent for use in treating a patient suffering from a heart condition, wherein said patient has a body mass index (BMI) of at most 40 kg/m², wherein said pharmaceutical composition is administered to said subject via inhalation.

Embodiment 235. An inhalable pharmaceutical composition comprising a therapeutically effective amount of an antiarrhythmic agent for use in treating a patient suffering from a heart condition, wherein said patient has a body mass index (BMI) of at most 40 kg/m².

Embodiment 236. The pharmaceutical composition of embodiment 234 or 235, wherein said patient has a body mass index (BMI) of at most 35 kg/m².

Embodiment 237. An aerosolized solution comprising a therapeutically effective amount of an antiarrhythmic agent for use in treating a patient suffering from a heart condition, wherein said patient has a body mass index (BMI) of at most 40 kg/m².

Embodiment 238. The aerosolized solution of embodiment 237, wherein said patient has a body mass index (BMI) of at most 35 kg/m².

Embodiment 239. A method of treating a heart condition, comprising:
identifying a human subject that has experienced or is experiencing said heart condition, and that has a body mass index (BMI) of no more than 40 kg/m²; and
administering to said subject via oral inhalation a pharmaceutical composition that comprises a therapeutically effective amount of an antiarrhythmic agent.

Embodiment 240. The method of embodiment 239, wherein said identifying comprises obtaining a measured body mass of said subject.

Embodiment 241. The method of embodiment 240, wherein said identifying comprises measuring body mass of said subject to obtain said measured body mass of said subject.

Embodiment 242. The method of embodiment 239, wherein said identifying comprises obtaining a measured body mass and a measured height of said subject.

Embodiment 243. The method of embodiment 242, wherein said identifying further comprises measuring said body mass and height of said subject to obtain said measured body mass and said measured height.

Embodiment 244. The method of embodiment 242 or 243, wherein said identifying further comprises calculating BMI of said subject based on said measured body mass and said measured height.

Embodiment 245. A method of treatment of a heart condition, comprising: administering to a human subject via inhalation a pharmaceutical composition that comprises a therapeutically effective amount of an antiarrhythmic agent, wherein said subject has been prescribed with said pharmaceutical composition based, at least in part, on that: (a) said subject has experienced or is experiencing said heart condition, and (b) said subject has a body mass index (BMI) of at most 40 kg/m².

Embodiment 246. The method of any one of embodiments 239-245, wherein said subject is determined to have a body mass of at most 100 kg, 95 kg, 90 kg, 85 kg, or 80 kg.

Embodiment 247. The method of any one of embodiments 239-245, wherein said subject is determined to have a body mass of at most 90 kg, 89 kg, 88 kg, 87 kg, 86 kg, 85 kg, 84 kg, 83 kg, 82 kg, 81 kg, or 80 kg.

Embodiment 248. The method of any one of embodiments 239-245, wherein said subject is determined to have a body mass of less than 90 kg, 89 kg, 88 kg, 87 kg, 86 kg, 85 kg, 84 kg, 83 kg, 82 kg, 81 kg, or 80 kg.

Embodiment 249. The method of any one of embodiments 239-248, wherein said subject is determined to have a BMI of at most 37 kg/m², 35 kg/m², 33 kg/m², 31 kg/m², 30 kg/m², 29 kg/m², 28 kg/m², 27 kg/m², 26 kg/m², or 25 kg/m².

Embodiment 250. The method of any one of embodiments 239-248, wherein said subject is determined to have a BMI of at most 32 kg/m², 31.5 kg/m², 31 kg/m², 30.5 kg/m², 30 kg/m², 29.5 kg/m², 29 kg/m², 28.5 kg/m², 28 kg/m², 27.5 kg/m², 27 kg/m², 26.5 kg/m², 26 kg/m², 25.5 kg/m², or 25 kg/m².

Embodiment 251. The method of any one of embodiments 239-248, wherein said subject is determined to have a BMI of less than 32 kg/m², 31.5 kg/m², 31 kg/m², 30.5 kg/m², 30 kg/m², 29.5 kg/m², 29 kg/m², 28.5 kg/m², 28 kg/m², 27.5 kg/m², 27 kg/m², 26.5 kg/m², 26 kg/m², 25.5 kg/m², or 25 kg/m².

Embodiment 252. The method of any one of embodiments 239-248, wherein said subject is determined to have a BMI of at most 35 kg/m².

Embodiment 253. A method of treating a human subject suffering from a heart condition, comprising:
obtaining a measured body mass index (BMI) of said subject; and
then administering to said subject via inhalation a pharmaceutical composition if said measured BMI is at most 40 kg/m²,
wherein said pharmaceutical composition comprises a therapeutically effective amount of an antiarrhythmic agent.

Embodiment 254. The method of embodiment 253, wherein said obtaining comprises measuring a body mass of said subject.

Embodiment 255. The method of embodiment 253, wherein said obtaining comprises obtaining said measured body mass and a measured height of said subject.

Embodiment 256. The method of embodiment 255, wherein said obtaining further comprises measuring weight and height of said subject to obtain said measured body mass and said measured height.

Embodiment 257. The method of embodiment 255 or 256, wherein said obtaining said measurement further comprises calculating said measured BMI based on said measured body mass and said measured height.

Embodiment 258. The method of any one of embodiments 253-257, wherein the method comprises administering to said subject via inhalation said pharmaceutical composition if said measured BMI is at most 37 kg/m², 35 kg/m², 33 kg/m², 31 kg/m², 30 kg/m², 29 kg/m², 28 kg/m², 27 kg/m², 26 kg/m², or 25 kg/m².

Embodiment 259. The method of any one of embodiments 253-257, wherein the method comprises administering to said subject via inhalation said pharmaceutical composition if said measured BMI is at most 32 kg/m², 31.5 kg/m², 31 kg/m², 30.5 kg/m², 30 kg/m², 29.5 kg/m², 29 kg/m², 28.5 kg/m², 28 kg/m², 27.5 kg/m², 27 kg/m², 26.5 kg/m², 26 kg/m², 25.5 kg/m², or 25 kg/m².

Embodiment 260. The method of any one of embodiments 253-257, wherein the method comprises administering to said subject via inhalation said pharmaceutical composition if said measured BMI is less than 32 kg/m², 31.5 kg/m², 31 kg/m², 30.5 kg/m², 30 kg/m², 29.5 kg/m², 29 kg/m², 28.5 kg/m², 28 kg/m², 27.5 kg/m², 27 kg/m², 26.5 kg/m², 26 kg/m², 25.5 kg/m², or 25 kg/m².

Embodiment 261. The method of any one of embodiments 253-257, wherein the method comprises administering to said subject via inhalation said pharmaceutical composition if said measured BMI is at most 35 kg/m².

Embodiment 262. The method of any one of embodiments 242-244 or 255-257, wherein said measured height, or said BMI is measured at the time of treating.

Embodiment 263. The method of any one of embodiments 239-262, wherein said subject has a systolic blood pressure that is greater than about 90 mmHg at the time of treating.

Embodiment 264. The method of any one of embodiments 239-263, wherein said subject has a systolic blood pressure that is from about 100 mmHg to about 160 mmHg at the time of treating.

Embodiment 265. The method of any one of embodiments 239-264, wherein said subject has a ventricular rate that is no more than 170 BPM at the time of treating.

Embodiment 266. The method of any one of embodiments 239-265, wherein said subject has a ventricular rate that is from about 80 BPM to about 155 BPM at the time of treating.

Embodiment 267. The method of any one of embodiments 239-266, wherein said antiarrhythmic agent comprises a class I, II, III, IV, or V antiarrhythmic agent.

Embodiment 268. The method of any one of embodiments 239-266, wherein said pharmaceutical composition comprises a therapeutically effective amount of flecainide or a pharmaceutically acceptable salt thereof.

Embodiment 269. The method of embodiment 268, wherein said pharmaceutical composition in the form of a liquid solution.

Embodiment 270. The method of embodiment 269, wherein said pharmaceutical composition has said salt of flecainide at a concentration that is at least 60 mg/mL.

Embodiment 271. The method of any one of embodiments 268-270, wherein said pharmaceutical composition further comprises a cyclodextrin.

Embodiment 272. The method of embodiment 271, wherein said cyclodextrin comprises hydroxypropyl-β-cyclodextrin.

Embodiment 273. The method of embodiment 271 or 272, wherein said concentration of said cyclodextrin is from about 15% (w/v) to about 25% (w/v) of said solution.

Embodiment 274. The method of embodiment 271 or 272, wherein said concentration of said cyclodextrin is at least about 10% (w/v) of said solution.

Embodiment 275. The method of embodiment 271 or 272, wherein said concentration of said cyclodextrin is about 20% (w/v) of said solution.

Embodiment 276. The method of any one of embodiments 268-275, wherein a pH of said solution is above 5.5 at room temperature.

Embodiment 277. The method of any one of embodiments 268-276, wherein said concentration of said salt of flecainide is about 65 mg/mL to about 130 mg/mL.

Embodiment 278. The method of any one of embodiments 268-276, wherein said concentration of said salt of flecainide is about 75 mg/mL.

Embodiment 279. The method of any one of embodiments 268-278, wherein said salt of flecainide is selected from the group consisting of: flecainide acetate, flecainide hydrochloride, flecainide citrate, flecainide phosphate, and flecainide nitrate.

Embodiment 280. The method of any one of embodiments 268-278, wherein said salt of flecainide comprises flecainide acetate.

Embodiment 281. The method of any one of embodiments 268-280, wherein said pharmaceutical composition further comprises an acid.

Embodiment 282. The method of embodiment 281, wherein said acid is selected from the group consisting of: acetic acid, citric acid, nitric acid, hydrochloric acid, sulfuric acid, maleic acid, tartaric acid, phosphoric acid, aconitic acid, adipic acid, ascorbic acid, benzoic acid, caprylic acid, cholic acid, formic acid, glutamic acid, lactic acid, propionic acid, sorbic acid, stearic acid, and succinic acid.

Embodiment 283. The method of embodiment 281 or 282, wherein a concentration of said acid in said pharmaceutical composition is about 2 mM to about 50 mM.

Embodiment 284. The method of embodiment 281 or 282, wherein said concentration of said acid is about 20 mM.

Embodiment 285. The method of embodiment 281 or 282, wherein said concentration of said acid is about 5 mM.

Embodiment 286. The method of any one of embodiments 281-285, wherein said acid comprises acetic acid.

Embodiment 287. The method of embodiment 286, wherein a concentration of acetic acid is about 5 mM.

Embodiment 288. The method of any one of embodiments 281-285, wherein said acid comprises a mixture of acids selected from the group consisting of: acetic acid, citric acid, nitric acid, hydrochloric acid, and sulfuric acid.

Embodiment 289. The method of any one of embodiments 276-288, wherein said pH of said solution is from about 5.5 to about 6.5.

Embodiment 290. The method of any one of embodiments 276-288, wherein said pH of said solution is about 5.9.

Embodiment 291. The method of any one of embodiments 268-290, wherein said pharmaceutical composition further comprises a sweetener.

Embodiment 292. The method of embodiment 291, wherein said sweetener comprises saccharin.

Embodiment 293. The method of embodiment 291, wherein said sweetener comprises saccharin sodium.

Embodiment 294. The method of any one of embodiments 268-293, wherein said administration results in a peak plasma concentration (Cₘₐₓ) of said salt of flecainide in said subject that is at least 200 ng/mL.

Embodiment 295. The method of embodiment 294, wherein said Cₘₐₓ is between about 250 ng/mL and about 1000 ng/mL.

Embodiment 296. The method of embodiment 294, wherein said Cₘₐₓ is between about 300 ng/mL and about 700 ng/ml.

Embodiment 297. The method of any one of embodiments 268-296, wherein about 100 mg to about 250 mg of said salt of flecainide is administered to said subject via inhalation.

Embodiment 298. The method of any one of embodiments 268-296, wherein about 90 mg of said salt of flecainide is administered to said subject via inhalation.

Embodiment 299. The method of any one of embodiments 268-296, wherein about 120 mg of said salt of flecainide is administered to said subject via inhalation.

Embodiment 300. The method of any one of embodiments 268-296, wherein about 200 mg of said salt of flecainide is administered to said subject via inhalation.

Embodiment 301. The method of any one of embodiments 239-300, wherein said administration of said pharmaceutical composition is performed within about 10 min.

Embodiment 302. The method of any one of embodiments 239-301, wherein said pharmaceutical composition is a nebulized solution that comprises nebulized droplets having a mass median aerodynamic diameter of less than 10 μm.

Embodiment 303. The method of any one of embodiments 239-302, wherein said administration of said pharmaceutical composition is performed via one or two inhalations.

Embodiment 304. The method of any one of embodiments 239-301, wherein said administration of said pharmaceutical composition is performed via two inhalations that are separated by a break for from about 10 seconds to about 1 minute.

Embodiment 305. The method of any one of embodiments 239-304, wherein said administration of said pharmaceutical composition is performed within about 5 min.

Embodiment 306. The method of any one of embodiments 239-305, wherein said administration is performed via a nebulizer.

Embodiment 307. The method of embodiment 306, wherein said nebulizer is a breath-actuated nebulizer.

Embodiment 308. The method of embodiment 306, wherein said nebulizer is a jet nebulizer.

Embodiment 309. The method of embodiment 306, wherein said nebulizer is a vibrating mesh nebulizer.

Embodiment 310. The method of any one of embodiments 239-309, wherein said heart condition comprises atrial arrhythmia.

Embodiment 311. The method of embodiment 310, wherein said atrial arrhythmia comprises tachycardia.

Embodiment 312. The method of embodiment 310 or 311, comprising acute treatment after detection of said atrial arrhythmia in said subject.

Embodiment 313. The method of any one of embodiments 239-312, wherein said heart condition comprises atrial fibrillation.

Embodiment 314. The method of embodiment 313, wherein said atrial fibrillation is recurrent atrial fibrillation.

Embodiment 315. The method of embodiment 313, wherein said atrial fibrillation is paroxysmal atrial fibrillation.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

1-315. (canceled)

316. A method of treating cardiac arrhythmia in a subject, wherein the method comprises:
(a) administering to said subject via oral inhalation a first dose of a pharmaceutical composition, wherein said pharmaceutical composition is an aqueous solution that comprises flecainide or a pharmaceutically acceptable salt thereof at a concentration that is at least 60 mg/mL;
(b) after the administering of (a), waiting for a period of time, wherein said period of time is at least about 10 minutes; and
(c) after the waiting, administering to said subject a second dose of said pharmaceutical composition via oral inhalation, wherein said second dose comprises said flecainide or salt thereof in an amount that is equivalent to about 40% to about 100% (w/w) of said first dose.

317. The method of claim 316, wherein said second dose comprises said flecainide or pharmaceutically acceptable salt thereof in an amount that is equivalent to about 40% to about 80% (w/w) of said first dose.

318. The method of claim 316, wherein said second dose comprises said flecainide or pharmaceutically acceptable salt thereof in an amount that is equivalent to said first dose.

319. The method of claim 316, wherein said pharmaceutical composition comprises flecainide acetate at a concentration of about 65 mg/mL to about 130 mg/mL.

320. The method of claim 316, wherein said pharmaceutical composition comprises flecainide acetate at a concentration of about 75 mg/mL.

321. The method of claim 316, wherein said cardiac arrhythmia comprises atrial arrhythmia.

322. The method of claim 316, wherein said cardiac arrhythmia comprises atrial fibrillation.

323. A method of treating cardiac arrhythmia, the method comprising:
(a) identifying a subject that: (i) is suffering from said cardiac arrhythmia, and (ii) has a qualifying body mass index (BMI), wherein said qualifying BMI is a BMI of no more than about 40 kg/m²;
(b) designating said subject as eligible for treatment at least on the basis that said subject has said qualifying BMI;
(c) administering to said subject via oral inhalation a pharmaceutical composition that comprises a therapeutically effective amount of flecainide or a pharmaceutically acceptable salt thereof in accordance with said designating.

324. The method of claim 323, wherein said subject has a systolic blood pressure that is greater than about 90 mmHg at initiation of said administering.

325. The method of claim 323, wherein said subject has a ventricular rate that is no more than 170 BPM at initiation of said administering.

326. The method of claim 323, wherein said subject has a ventricular rate that is from about 80 BPM to about 155 BPM at initiation of said administering.

327. The method of claim 323, wherein said qualifying BMI is a BMI of no more than about 35 kg/m².

328. The method of claim 323, wherein said pharmaceutical composition comprises flecainide acetate at a concentration of about 65 mg/mL to about 130 mg/mL.

329. The method of claim 323, wherein said pharmaceutical composition comprises flecainide acetate at a concentration of about 75 mg/mL.

330. The method of claim 328, wherein about 100 mg to about 250 mg of flecainide acetate is administered to said subject via inhalation.

331. The method of claim 323, wherein said administration is performed via a nebulizer.

332. The method of claim 331, wherein said nebulizer is a vibrating mesh nebulizer.

333. The method of claim 323, wherein said cardiac arrhythmia comprises atrial fibrillation.

334. A kit comprising a first dose and a second dose of a pharmaceutical composition formulated for oral inhalation, wherein:
(a) said pharmaceutical composition comprises flecainide or a pharmaceutically acceptable salt thereof;
(b) said second dose comprises said flecainide or a pharmaceutically acceptable salt thereof in an amount that is equivalent to about 40% to about 80% (w/w) of said first dose;
(c) said first dose is provided in a first vessel, and said second dose is provided in a second vessel.

335. The kit of claim 334, wherein said pharmaceutical composition comprises flecainide acetate at a concentration that is at least 60 mg/mL.
